(19) United States
(12) Patent Application Publication
(10) Pub. No.: US 2026/0325690 A1
(43) Pub. Date: Oct. 22, 2026

(54) ANTI-ROR2 ANTIBODY-RADIONUCLIDE CONJUGATES

(71) Applicant: Solve Therapeutics, Inc., San Diego, CA (US)

(72) Inventors: Jeffrey L. SCHAAL, Durham, NC (US); Benjamin GIGLIO, Chapel Hill, NC (US); Riccardo MUZZIOLI, Durham, NC (US); Darryl McDOUGALD, Durham, NC (US)

(73) Assignee: Solve Therapeutics, Inc., San Diego, CA (US)

(21) Appl. No.: 19/260,024

(22) Filed: Jul. 3, 2025

Related U.S. Application Data
(60) Provisional application No. 63/667,681, filed on Jul. 3, 2024.

(51) Int. Cl. A61K 51/10; A61K 45/06; C07K 16/28

(52) U.S. Cl. CPC A61K 51/1093; A61K 45/06; A61K 51/1027; C07K 16/2803; A61K 2121/00; A61K 2123/00

(57) ABSTRACT

The present disclosure provides antibody-radionuclide conjugates comprising an anti-ROR2 antibody or an antigen-binding portion thereof and a radioactive moiety, and methods of using them to diagnose and treat ROR2-expressing cancers.

### CROSS-REFERENCE TO RELATED APPLICATION

This application claims priority to U.S. Provisional Patent Application No. 63/667,681, filed Jul. 3, 2024. The disclosure of the priority application is incorporated by reference herein in its entirety.

### SEQUENCE LISTING

The instant application contains a Sequence Listing which has been submitted electronically in XML format and is hereby incorporated by reference herein in its entirety. The electronic copy of the Sequence Listing, created on Jun. 26, 2025, is named 122878.US019.xml and is 37,984 bytes in size.

### BACKGROUND OF THE INVENTION

Cancer is the second leading cause of human death next to heart disease. Receptor tyrosine kinases (RTKs) play a key role in oncogenic transformation, as well as cancer growth and metastasis, by regulating cell differentiation, proliferation, migration, angiogenesis, and survival. Receptor tyrosine kinase-like orphan receptor 2 (“ROR2”) is a cell membrane protein and is a receptor for Wnt5a, a proinflammatory factor in human ovarian granulosa cells. ROR2 modulates Wnt signaling through sequestration of Wnt ligands and can also repress transcription of Wnt target genes involved in tumor suppression. ROR2 has been implicated in the progression of numerous cancers, including breast, ovarian, pancreatic, cervical, gastric, renal, head and neck, bone, skin, and prostate cancers. Accordingly, ROR2 is of interest as a target for anti-cancer immunotherapies.

In view of the role of ROR2 in cancer, there is a need for new and improved therapies that target ROR2-positive cancer cells.

### SUMMARY OF THE INVENTION

The present disclosure provides antibody-radionuclide conjugates (ARCs, or immunoconjugates) that comprise an anti-ROR2 antibody or antigen-binding portion thereof conjugated (e.g., by way of a radiolabeling prosthetic linker) to a radioactive isotope (e.g., ¹¹C, ¹³N ¹⁸F, ³²P, ³³P, ⁶⁴Cu, ⁶⁷Cu, ⁶⁸Ga ⁷⁵Br, ⁷⁶Br, ⁷⁷Br, ⁷⁸Br, ⁸³Sr, ⁸⁹Sr, ⁸⁶Y, ⁹⁰Y, ⁸⁹Zr, ^{99m}Tc, ¹⁰⁵Rh ¹¹¹In, ¹²³I, ¹²⁴I, ¹²⁵I, ¹³¹I, ¹³³Xe, ¹⁵³Sm, ¹⁴⁹Tb, ¹⁵²Tb, ¹⁵⁵Tb, ¹⁶¹Tb, ¹⁶⁵Dy, ¹⁶⁶Ho, ¹⁷⁷Lu, ¹⁸⁶Rh ¹⁹⁹Au, ²⁰¹Tl, ²⁰³Pb, ²⁰⁹Pb, ²¹²Pb, ²¹²Bi, ²¹³Bi, ²¹¹At, ²²³Ra, ²²⁴Ra, ²²⁵Ac, and ²²⁷Th).

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab is an anti-ROR2 antibody or antigen-binding portion.

In some embodiments of an immunoconjugate herein, the antibody or antigen-binding portion of the immunoconjugate competes or cross-competes for binding to human ROR2, or binds to the same human ROR2 epitope, as an antibody that comprises a heavy chain (HC) and a light chain (LC) comprising SEQ ID NOs: 1 and 2, respectively; SEQ ID NOs: 11 and 2, respectively; SEQ ID NOs: 15 and 16, respectively; or SEQ ID NOs: 21 and 16, respectively.

In certain embodiments, the antibody or antigen-binding portion comprises heavy chain complementarity-determining region (CDR) 1-3 (HCDR1-3) and light chain CDR1-3 (LCDR1-3) amino acid sequences of SEQ ID NOs: 5, 6, 7, 8, 9, and 10, respectively; SEQ ID NOs: 13, 6, 14, 8, 9, and 10, respectively; or SEQ ID NOs: 19, 6, 20, 8, 9, and 10, respectively.

In certain embodiments, the antibody or antigen-binding portion comprises HCDR1 comprising SEQ ID NO: 40; HCDR2 comprising SEQ ID NO: 28; HCDR3 comprising SEQ ID NO: 24; LCDR1 comprising SEQ ID NO: 41; LCDR2 comprising SEQ ID NO: 9; and LCDR3 comprising SEQ ID NO: 37. The antibody or antigen-binding portion may comprise, for example, HCDR1-3 and LCDR1-3 of 5, 6, 7, 8, 9, and 10, respectively, or HCDR1-3 and LCDR1-3 of 22, 23, 24, 25, 26, and 10, respectively.

In certain embodiments, the antibody or antigen-binding portion comprises heavy chain variable domain (VH) and light chain variable domain (VL) amino acid sequences of SEQ ID NOs: 3 and 4, respectively; SEQ ID NOs: 12 and 4, respectively; or SEQ ID NOs: 17 and 18, respectively.

In some embodiments, the antibody is of isotype IgG. For example, the antibody may be of isotype subclass IgG₁, IgG₂, IgG₃, or IgG₄. In certain embodiments, the Fc region of the antibody comprises one or more mutations that reduce effector function.

In particular embodiments, the antibody comprised by an immunoconjugate herein comprises HC and LC amino acid sequences of SEQ ID NOs: 1 and 2, respectively; SEQ ID NOs: 11 and 2, respectively; SEQ ID NOs: 15 and 16, respectively; or SEQ ID NOs: 21 and 16, respectively; optionally wherein the HC amino acid sequence lacks the C-terminal lysine.

In some embodiments, the antigen-binding portion comprised by an immunoconjugate herein is a Fab, F(ab′)₂, scFv, F(ab)₃, minibody, scFv-Fc, IgG-scFv, Fab-Fc, tandem scFv, triabody, tetrabody, Dab-Fc, diabody, sdAb, tandem sdAb, derived VHH or an sFv.

The present disclosure also provides a pharmaceutical composition comprising an immunoconjugate herein and a pharmaceutically acceptable excipient. In some embodiments, the pharmaceutical composition may further comprise an additional therapeutic agent, such as an immunomodulatory agent, a chemotherapeutic agent, a radionuclide agent, a nuclear imaging agent, an anti-neoplastic agent, or an anti-angiogenic agent.

The present disclosure also provides a method of treating cancer in a human patient in need thereof, comprising administering to the patient a therapeutically effective amount of an immunoconjugate herein. In some embodiments, the cancer expresses ROR2. In some embodiments, the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma. The method may further comprise administering to the patient an additional therapeutic agent, for example, an immunomodulatory agent, a chemotherapeutic agent, an anti-neoplastic agent, an anti-angiogenic agent, or a tumor vaccine.

The present disclosure also provides an immunoconjugate herein, or a pharmaceutical composition herein, for use in treating cancer in a method described herein.

The present disclosure also provides use of an immunoconjugate herein, or a pharmaceutical composition herein, for treating cancer in a method described herein.

The present disclosure also provides use of an immunoconjugate herein, or a pharmaceutical composition herein, in the manufacture of a medicament for treating cancer in a method described herein.

The present disclosure also provides an immunoconjugate herein, or a pharmaceutical composition herein, for use in the medical imaging of cancer in a method described herein.

The present disclosure also provides use of an immunoconjugate herein, or a pharmaceutical composition herein, for the medical imaging of cancer in a method described herein.

It is understood that the present disclosure also provides an ARC or pharmaceutical composition described herein for use in treating a patient (e.g., a human patient) in need thereof in a therapeutic method described herein. Also provided are uses of an ARC or pharmaceutical composition described herein for the manufacture of a medicament for treating a patient (e.g., a human patient) in need thereof in a therapeutic method described herein.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a graph showing the stability of [Zr89]-DFO-Ab1 over time in either 10 mM Acetate buffer or 250 mM Acetate buffer, measured by radio thin layer chromatography (radioTLC).

FIG. 2 is a set of radio high-pressure liquid chromatography-size exclusion chromatography (radioHPLC-SEC) graphs showing the formation of the ARC-ROR2 complex upon incubation of the ARC with antigen at various ratios of ROR2-His₆:mAb.

FIG. 3 is a set of PET/CT images showing the biodistribution and non-specific tumor uptake of [Zr89]-DFO-Ab1 in mice over time.

FIG. 4 is a bar graph depicting measured ROR2 expression levels in NCI-H1155, HCT-116, NCI-H520 and AsPc-1 cell lines.

FIG. 5A is a line graph showing nanomolar binding affinity of DFO-Ab1 to the ROR2-positive NCI-H1155 cell line.

FIG. 5B is a line graph showing percent cell internalization of DFO-Ab1 in the ROR2-positive NCI-H1155 cell line over 4 hours.

FIG. 6A is a line graph showing nanomolar binding affinity of DFO-Ab1 to the ROR2-positive NCT-116 cell line.

FIG. 6B is a line graph showing percent cell internalization of DFO-Ab1 in the ROR2-positive NCT-116 cell line over 4 hours.

FIG. 7 is a line graph showing low binding affinity of DFO-Ab1 to the ROR2-negative AsPc-1 cell line.

FIGS. 8A, 8B and 8C are a set of graphs showing dose-dependent, nanomolar binding affinity of [Zr89]-DFO-Ab1 upon radiolabeling with Zr-89 in ROR2-positive NCI-H1155, HCT-116 and NCI-H520 cell lines across a range of specific activities (SA). “CPM” refers to counts per minute.

FIG. 9A is an image showing non-specific uptake of [Zr89]-DFO-Ab1 in ROR2-negative xenograft (AsPc-1 tumors in female athymic mice) by PET/CT imaging.

FIG. 9B is an image showing highly ROR2-specific uptake of [Zr89]-DFO-Ab1 in ROR2-positive xenograft (NCI-H1155 tumors in female athymic mice) by PET/CT imaging.

FIG. 10A and FIG. 10B are a set of bar graphs showing low level of non-specific accumulation of [Zr89]-DFO-Ab1 in healthy tissues via serial tissue biodistribution analysis in naïve male and female mice, respectively.

FIG. 11 is a line graph showing an effective radioactive half-life of [Zr89]-DFO-Ab1 in whole blood pharmacokinetic analysis in male and female mice.

FIG. 12 is a set of bar graphs showing low Zr-89 uptake in various bone tissues, demonstrating strong stability of the DFO-Zr-89 chelate in vivo.

### DETAILED DESCRIPTION OF THE INVENTION

The present disclosure provides antibody-radionuclide conjugates (ARCs) comprising an antibody or an antigen-binding portion thereof that specifically binds to ROR2 (“ROR2 binders”). These ARCs can be used for diagnostic imaging and radiation therapy (e.g., to treat cancer) in a subject in need thereof (e.g., a human patient). For example, diagnostic anti-ROR2 ARCs may be used to identify or locate ROR2-positive cancer through positron emission tomography (PET) or single photon emission computed tomography (SPECT) imaging and help select treatment of such cancer, e.g., treatment that uses an anti-ROR2 antibody-drug conjugate (ADC), and track ROR2 expression in cancer during the course of a cancer treatment. Therapeutic anti-ROR2 ARCs may be used to treat cancer by targeting radioactive isotopes to ROR2-positive cancer cells.

Conjugation of radioisotopes to an antibody or antigen-binding fragment thereof targeting a tumor antigen such as ROR2 results in greater uptake of radioactivity in the targeted cancer cells, higher retention of radioactivity in the targeted cells after internalization, and less uptake of the radioactivity in normal cells. For example, there is less thyroid and renal uptake of the radioactivity. An advantage of targeted radiotherapy is that one can select a radioisotope with properties that are best matched to the constraints of the intended clinical application (e.g., diagnostic versus therapeutic application).

An “antibody-radio conjugate,” “antibody-radionuclide conjugate,” or “ARC,” “radionuclide antibody conjugate” or “RAC,” or “immunoconjugate” is used interchangeably herein and refers herein to an antibody or an antigen-binding portion thereof that is covalently or non-covalently bonded, with or without a radiolabeling prosthetic linker, to one or more radioactive isotope(s). It is understood that where the present disclosure refers to an anti-ROR2 antibody or an antigen-binding portion thereof, any moiety that serves as any means for binding to ROR2 may be used.

Embodiments of the antibody or portion thereof, radionuclides, and the radiolabeling prosthetic linker used in the ARCs are described in further detail below.

### I. Anti-ROR2 Antibodies

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (V_{H}) and a heavy chain constant region (C_{H}). Each light chain is composed of a light chain variable domain (V_{L}) and a light chain constant region (C_{L}). The V_{H} and V_{L} domains can be subdivided further into regions of hypervariability, termed “complementarity determining regions” (CDRs), interspersed with regions that are more conserved, termed “framework regions” (FRs). Each V_{H} and V_{L} is composed of three CDRs (HCDR herein designates a CDR from the heavy chain; and LCDR herein designates a CDR from the light chain) and four FRs, arranged from amino-terminus to carboxyl-terminus in the following order: FR1, CDR1, FR2, CDR2, FR3, CDR3, FR4. The amino acid position numbers and FRs and CDRs in the heavy or light chain may be defined in accordance with the IMGT® system (Lefranc et al., Dev Comp Immunol. (2003) 27(1):55-77); or the Kabat system (Sequences of Proteins of Immunological Interest (National Institutes of Health, Bethesda, MD (1987 and 1991)); the Chothia system (Chothia & Lesk, J Mol Biol. (1987) 196:901-17; Chothia et al., Nature (1989) 342:878-83); Abhinandan et al., Molecular Immunology (2008) 45(14):3832-39; MacCallum et al., J Mol Biol. (1996) 262:732-45; or Honegger and Plückthun, J Mol Biol. (2001) 309(3):657-70.

The term “recombinant antibody” refers to a non-naturally occurring antibody that is expressed from a cell or cell line comprising one or more nucleotide sequences that encode the antibody, wherein the cell or cell line does not naturally comprise the nucleotide sequence(s).

The term “isolated protein,” “isolated polypeptide” or “isolated antibody” refers to a protein, polypeptide or antibody that by virtue of its origin or source of derivation (1) is not associated with components that naturally accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between two molecules, e.g., an antigen and an antibody. The intrinsic attractiveness of an antibody for an antigen is typically expressed as the binding affinity equilibrium constant (K_{D}) of the antibody-antigen interaction. An antibody is said to specifically bind to an antigen when the K_{D} for the binding is ≤1 μM, e.g., ≤100 nM or ≤10 nM. A K_{D} binding affinity constant can be measured, e.g., by surface plasmon resonance (SPR) using, for example, the Biacore™ T200 system, the IBIS-MX96 SPR system from IBIS Technologies, or the Carterra LSA SPR platform, or by bio-layer interferometry (BLI) using, for example using the Octet™ system from ForteBio.

The term “epitope” as used herein refers to a portion (determinant) of an antigen that specifically binds to an antibody or a related molecule such as a bispecific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., ROR2) or a relevant portion thereof, then screening for binding to the epitope. An antibody to a particular epitope also may be generated using phage display methods.

One can determine whether an antibody binds to the same epitope as or competes for binding with an anti-ROR2 antibody of the present disclosure by using methods known in the art, including, without limitation, competition assays, epitope binning, and alanine scanning. In some embodiments, one allows the anti-ROR2 antibody of the present disclosure to bind to ROR2 under saturating conditions, and then measures the ability of the test antibody to bind to ROR2. If the test antibody is able to bind to ROR2 at the same time as the reference anti-ROR2 antibody, then the test antibody binds to a different epitope than the reference anti-ROR2 antibody. However, if the test antibody is not able to bind to ROR2 at the same time, then the test antibody may bind to the same epitope, an overlapping epitope, or an epitope that is in close proximity to the epitope bound by the anti-ROR2 antibody of the present disclosure. This experiment can be performed using, e.g., ELISA, RIA, Biacore™ SPR, BLI, or flow cytometry. To test whether an anti-ROR2 antibody cross-competes with another anti-ROR2 antibody, one may use the competition method described above in two directions, i.e., determining if the known antibody blocks the test antibody and vice versa. Such cross-competition experiments may be performed, e.g., using a Biacore™ T200, IBIS MX96, or Carterra LSA SPR instrument or the Octet™ system.

The term “antigen-binding portion” or “antigen-binding fragment” of an antibody, as used herein, refers to one or more portions or fragments of an antibody that retain the ability to specifically bind to the antigen (e.g., human ROR2, or a portion thereof) of the antibody. It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, C_{L} and C_{H1} domains; (ii) a F(ab′)₂ fragment: a bivalent fragment comprising two Fab fragments linked by a disulfide bridge at the hinge region; (iii) an Fd fragment consisting of the V_{H} and C_{H1} domains; (iv) an Fv fragment consisting of the V_{L} and V_{H} domains of a single arm of an antibody, (v) a dAb fragment, which consists of a V_{H} domain; and (vi) an isolated CDR capable of specifically binding to an antigen. Furthermore, although the two domains of the Fv fragment, V_{L} and V_{H}, are encoded by separate genes, they can be joined, using recombinant methods, by a synthetic linker that enables them to be made as a single polypeptide chain in which the V_{L} and V_{H} domains pair to form monovalent molecules (known as single chain Fv (scFv)). Additionally, the present disclosure includes the structure of two single-chain variable fragments (scFv) can be linked to the CH3 domain to form a stable ‘minibody’. The scFv fragments can be recombinantly engineered for display on the Fc fragment, termed an scFv-Fc format, to improve tissue penetration, or recombinantly engineered to the C-terminus of the antibody's IgG structure to enable bispecific receptor engagement and multivalency. Also within the present disclosure are antigen-binding molecules comprising a V_{H} and/or a V_{L}. In the case of a V_{H}, the molecule may also comprise one or more of a C_{H1}, hinge, C_{H2}, or C_{H3} region and may be joined in tandem as a multivalent V_{H} protein. Such single chain antibodies are also intended to be encompassed within the term “antigen-binding portion” of an antibody. Other forms of single chain antibodies, such as diabodies, are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesin molecules can be obtained using standard recombinant DNA techniques, e.g., as described herein.

The class (isotype) and subclass of anti-ROR2 antibodies may be determined by any method known in the art. In general, the class and subclass of an antibody may be determined using antibodies that are specific for a particular class and subclass of antibody. Such antibodies are available commercially. The class and subclass can be determined by ELISA or Western blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies.

In some embodiments, the constant region of the anti-ROR2 antibody herein may comprise mutations that improve the therapeutic potential of the antibody, such as mutations that reduce or eliminate effector functions of the antibody. For example, an antibody may comprise a human IgG1 constant region with the L235E mutation, the P329A mutation, the “LALA” mutations (L234A/L235A), the “LALAPA” mutations (L234A/L235A/P329A), the “LALAGA” mutations (L234A/L235A/G237A), the “LALAGR” mutations (L234A/L235A/G236R), and/or the “LALAPG” mutations (L234A/L235A/P329G) (Eu numbering). Further, for example, the monospecific or multispecific antibody herein may comprise a human IgG4 constant region with the mutation L235E and/or the mutation S228P (Eu numbering). An IgG constant region may comprise mutations that improve the serum half-life of the antibody (e.g., the “YTE” mutations) and/or improve manufacturing and yield of the antibody.

An ARC of the invention comprises an antibody or an antigen-binding portion thereof that specifically binds to ROR2. Unless otherwise stated, “ROR2” refers to human ROR2. A human ROR2 polypeptide sequence is available under UniProt Accession No. Q01974 (ROR2 HUMAN), as shown below:

| (SEQ ID NO: 38) | |
|---|---|
| 1 | MARGSALPRR PLLCIPAVWA AAALLLSVSR TSGEVEVLDP NDPLGPLDGQ |
| 51 | DGPIPTLKGY FLNFLEPVNN ITIVQGQTAI LHCKVAGNPP PNVRWLKNDA |
| 101 | PVVQEPRRII IRKTEYGSRL RIQDLDTTDT GYYQCVATNG MKTITATGVL |
| 151 | FVRLGPTHSP NHNFQDDYHE DGFCQPYRGI ACARFIGNRT IYVDSLQMQG |
| 201 | EIENRITAAF TMIGTSTHLS DQCSQFAIPS FCHFVFPLCD ARSRTPKPRE |
| 251 | LCRDECEVLE SDLCRQEYTI ARSNPLILMR LQLPKCEALP MPESPDAANC |
| 301 | MRIGIPAERL GRYHQCYNGS GMDYRGTAST TKSGHQCQPW ALQHPHSHHL |
| 351 | SSTDFPELGG GHAYCRNPGG QMEGPWCFTQ NKNVRMELCD VPSCSPRDSS |
| 401 | KMGILYILVP SIAIPLVIAC LFFLVCMCRN KQKASASTPQ RRQLMASPSQ |
| 451 | DMEMPLINQH KQAKLKEISL SAVREMEELG EDREGKVYKG HLFGPAPGEQ |
| 501 | TQAVAIKTLK DKAEGPLREE FRHEAMLRAR LQHPNVVCLL GVVTKDQPLS |
| 551 | MIFSYCSHGD LHEFLVMRSP HSDVGSTDDD RTVKSALEPP DEVHLVAQIA |
| 601 | AGMEYLSSHH VVHKDLATRN VLVYDKLNVK ISDLGLFREV YAADYYKLLG |
| 651 | NSLLPIRWMA PEAIMYGKES IDSDIWSYGV VLWEVESYGL QPYCGYSNQD |
| 701 | VVEMIRNRQV LPCPDDCPAW VYALMIECWN EFPSRRPREK DIHSRLRAWG |
| 751 | NLSNYNSSAQ TSGASNTTQT SSLSTSPVSN VSNARYVGPK QKAPPEPQPQ |
| 801 | FIPMKGQIRP MVPPPQLYVP VNGYQPVPAY GAYLPNFYPV QIPMQMAPQQ |
| 851 | VPPQMVPKPS SHHSGSGSTS TGYVTTAPSN TSMADRAALL SEGADDTQNA |
| 901 | PEDGAQSTVQ EAEEEEEGSV PETELLGDCD TLQVDEAQVQ LEA |

In the above sequence, the extracellular domain spans amino acids 34-403. The anti-ROR2 antibodies herein bind to an epitope in the extracellular domain.

Amino acid sequences of exemplary anti-ROR2 antibodies used in the ARCs of the invention are shown in Table 1 below.

TABLE 1
SEQ ID NOs of Exemplary Anti-ROR2 Antibodies

| Ab | HC | LC | VH | VL | HCDR1 | HCDR2 | HCDR3 | LCDR1 | LCDR2 | LCDR3 |
|---|---|---|---|---|---|---|---|---|---|---|
| 1 | 1 | 2 | 3 | 4 | 5 | 6 | 7 | 8 | 9 | 10 |
| 2 | 11 | 2 | 12 | 4 | 13 | 6 | 14 | 8 | 9 | 10 |
| 3 | 15 | 16 | 17 | 18 | 19 | 6 | 20 | 8 | 9 | 10 |
| 4 | 21 | 16 | 17 | 18 | 19 | 6 | 20 | 8 | 9 | 10 |

Ab1 and Ab2 are humanized, effectorless versions of murine anti-ROR2 antibody 6E6 (see, e.g., PCT Patent Publication WO 2021/102055 and PCT Patent Application PCT/US2023/086552). Ab3 is a chimeric version of 6E6. Ab4 is a chimeric, effectorless version of 6E6.

In some embodiments, an anti-ROR2 antibody or antigen-binding portion thereof used in an ARC of the present disclosure competes or cross-competes for binding to human ROR2 with, or binds to the same epitope of human ROR2 as, an antibody comprising:
a) a heavy chain (HC) comprising the amino acid sequence of SEQ ID NO: 1 and a light chain (LC) comprising the amino acid sequence of SEQ ID NO: 2;
b) an HC comprising the amino acid sequence of SEQ ID NO: 11 and an LC comprising the amino acid sequence of SEQ ID NO: 2;
c) an HC comprising the amino acid sequence of SEQ ID NO: 15 and an LC comprising the amino acid sequence of SEQ ID NO: 16; or
d) an HC comprising the amino acid sequence of SEQ ID NO: 21 and an LC comprising the amino acid sequence of SEQ ID NO: 16.

In some embodiments, the anti-ROR2 antibody or antigen-binding portion has HCDR1-3 comprising the amino acid sequences of
SEQ ID NOs: 5, 6, and 7, respectively;
SEQ ID NOs: 13, 6, and 14, respectively; or
SEQ ID NOs: 19, 6, and 20, respectively.

In some embodiments, the anti-ROR2 antibody or antigen-binding portion has a heavy chain variable domain (VH) amino acid sequence that is at least 80%, 85%, 90%, 91%, 92%, 93%, 94%, 95%, 96%, 97%, 98%, or 99% identical to the amino acid sequence of SEQ ID NO: 3, 12, or 17.

In some embodiments, the anti-ROR2 antibody or antigen-binding portion has a VH comprising the amino acid sequence of SEQ ID NO: 3, 12, or 17.

In some embodiments, the anti-ROR2 antibody has an HC amino acid sequence that is at least 80%, 85%, 90%, 91%, 92%, 93%, 94%, 95%, 96%, 97%, 98%, or 99% identical to the amino acid sequence of SEQ ID NO: 1, 11, 15, or 21, or said sequence without the C-terminal lysine.

In some embodiments, the anti-ROR2 antibody comprises an HC amino acid sequence of SEQ ID NO: 1, 11, 15, or 21, or said sequence without the C-terminal lysine.

In some embodiments, the anti-ROR2 antibody or antigen-binding portion has LCDR1-3 comprising the amino acid sequences of SEQ ID NOs: 8-10, respectively.

In some embodiments, the anti-ROR2 antibody or antigen-binding portion has a light chain variable domain (VL) amino acid sequence that is at least 80%, 85%, 90%, 91%, 92%, 93%, 94%, 95%, 96%, 97%, 98%, or 99% identical to the amino acid sequence of SEQ ID NO: 4 or 18.

In some embodiments, the anti-ROR2 antibody or antigen-binding portion has a VL comprising the amino acid sequence of SEQ ID NO: 4 or 18.

In some embodiments, the anti-ROR2 antibody has an LC amino acid sequence that is at least 80%, 85%, 90%, 91%, 92%, 93%, 94%, 95%, 96%, 97%, 98%, or 99% identical to the amino acid sequence of SEQ ID NO: 2 or 16.

In some embodiments, the anti-ROR2 antibody comprises an LC amino acid sequence of SEQ ID NO: 2 or 16.

In certain embodiments, the anti-ROR2 antibody or antigen-binding portion comprises any of the above-described heavy chain sequences paired with any one of the above-described light chain sequences.

In some embodiments, the anti-ROR2 antibody or antigen-binding portion of the present disclosure comprises the HCDR1-3 and LCDR1-3 amino acid sequences of:
a) SEQ ID NOs: 5, 6, 7, 8, 9, and 10, respectively;
b) SEQ ID NOs: 13, 6, 14, 8, 9, and 10, respectively; or
c) SEQ ID NOs: 19, 6, 20, 8, 9, and 10, respectively.

In some embodiments, the anti-ROR2 antibody or antigen-binding portion of the present disclosure comprises a VH and a VL that are at least 80%, 85%, 90%, 91%, 92%, 93%, 94%, 95%, 96%, 97%, 98%, or 99% identical (e.g., at least 90% identical) to the amino acid sequences of:
a) SEQ ID NOs: 3 and 4, respectively;
b) SEQ ID NOs: 12 and 4, respectively; or
c) SEQ ID NOs: 17 and 18, respectively.

In some embodiments, the anti-ROR2 antibody or antigen-binding portion of the present disclosure comprises a VH and a VL that comprise the amino acid sequences of:
a) SEQ ID NOs: 3 and 4, respectively;
b) SEQ ID NOs: 12 and 4, respectively; or
c) SEQ ID NOs: 17 and 18, respectively.

In some embodiments, the anti-ROR2 antibody of the present disclosure comprises an HC and an LC that comprise the amino acid sequences of:
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively;
   optionally wherein the HC amino acid sequence is without the C-terminal lysine.

The present disclosure also provides an anti-ROR2 antibody or an antigen-binding portion thereof that competes or cross-competes for binding to human ROR2 with, or binds to the same epitope of human ROR2 as, any one of Ab1-Ab4.

In some embodiments, the anti-ROR2 antibody or antigen-binding portion comprises
HCDR1 comprising SEQ ID NO: 40;
HCDR2 comprising SEQ ID NO: 28; and
HCDR3 comprising SEQ ID NO: 24;
   and/or
LCDR1 comprising SEQ ID NO: 41;
LCDR2 comprising SEQ ID NO: 9; and
LCDR3 comprising SEQ ID NO: 37.

In some embodiments, the anti-ROR2 antibody or antigen-binding portion of the present disclosure comprises the HCDR1-3 and LCDR1-3 amino acid sequences of any one of Ab1-Ab4. The assignment of CDR regions may be in accordance with any method known in the art, such as IMGT®, Kabat, Chothia, Martin, Contact, or AHo definitions, or any combination of any of these definitions (Kabat plus Chothia, for example). Examples of CDR definitions under different methods is shown below for Ab1 (SEQ: SEQ ID NO):

Ab1 HCDRs

| Definition | HCDR1 | SEQ | HCDR2 | SEQ | HCDR3 | SEQ |
|---|---|---|---|---|---|---|
| IMGT ® | GFTFSTYG | 5 | ISSGGGYT | 6 | ARHPRDESYALDY | 7 |
| Kabat | TYGVS | 22 | TISSGGGYTHYAGSVKG | 23 | HPRDFSYALDY | 24 |
| Chothia | GFTESTY | 27 | SSGGGY | 28 | HPRDFSYALDY | 24 |
| AHo | AASGFTFSTYGVS | 29 | TISSGGGYTH | 30 | ARHPRDESYALDY | 7 |
| Contact | STYGVS | 32 | WVSTISSGGGYTH | 33 | ARHPRDESYALD | 34 |

Ab1 LCDRs

| Definition | LCDR1 | SEQ | LCDR2 | SEQ | LCDR3 | SEQ |
|---|---|---|---|---|---|---|
| IMGT ® | QDVGHY | 8 | WAS | 9 | QQYNIYPWT | 10 |
| Kabat | RASQDVGHYLA | 25 | WASTRAT | 26 | QQYNIYPWT | 10 |
| Chothia | RASQDVGHYLA | 25 | WASTRAT | 26 | QQYNIYPWT | 10 |
| AHo | RASQDVGHYLA | 25 | YWASTRAT | 31 | QQYNIYPWT | 10 |
| Contact | GHYLAWY | 35 | LLIYWASTRA | 36 | QQYNIYPW | 37 |

Thus, for example, the Ab1 IMGT®-defined HCDR1-3 and LCDR1-3 sequences of SEQ ID NOs: 5-10, respectively, may be replaced in any embodiment described herein by
SEQ ID NOs: 22, 23, 24, 25, 26, and 10, respectively;
SEQ ID NOs: 27, 28, 24, 25, 26, and 10, respectively;
SEQ ID NOs: 29, 30, 7, 25, 31, and 10, respectively; or
SEQ ID NOs: 32, 33, 34, 35, 36, and 37, respectively.
   Also contemplated is a set of Ab1 CDRs wherein each of HCDR1, HCDR2, HCDR3, LCDR1, LCDR2, and LCDR3 may individually be specified according to any of the methods for defining Ab1 CDRs as shown above (e.g., HCDR1 specified by the Kabat definition, HCDR2 specified by the Chothia definition, etc.). For example, in some embodiments, the anti-ROR2 antibody or antigen-binding portion thereof comprises:
an HCDR1 comprising SEQ ID NO: 5, 22, 27, 29, or 32;
an HCDR2 comprising SEQ ID NO: 6, 23, 28, 30, or 33;
an HCDR3 comprising SEQ ID NO: 7, 24, or 34;
an LCDR1 comprising SEQ ID NO: 8, 25, or 35;
an LCDR2 comprising SEQ ID NO: 9, 26, 31, or 36; and/or
an LCDR3 comprising SEQ ID NO: 10 or 37.

The same means for defining Ab1 CDRs are contemplated for any of Ab2-Ab4.

In some embodiments, the anti-ROR2 antibody or antigen-binding portion of the present disclosure comprises a V_{H} and a V_{L} that are at least 80%, 85%, 90%, 91%, 92%, 93%, 94%, 95%, 96%, 97%, 98%, or 99% identical in amino acid sequence to the V_{H} and V_{L}, respectively, of any one of Ab1-Ab4.

In some embodiments, the anti-ROR2 antibody or antigen-binding portion of the present disclosure comprises a V_{H} and a V_{L} that are the V_{H} and V_{L}, respectively, of any one of Ab1-Ab4.

In some embodiments, the anti-ROR2 antibody is any one of Ab1-Ab4, or an antibody with the same amino acid sequences as said antibody.

In some embodiments, the anti-ROR2 antibody or antigen-binding portion is a variant antibody or antigen-binding portion. A “variant” antibody or antigen-binding portion has amino acid substitutions (which may be conservative or non-conservative) from a reference antibody or antigen-binding portion but does not have substantially altered biologic activity as compared to the reference antibody or antigen-binding portion. For example, the variant antibody or antigen-binding portion may retain at least 50%, 70%, 75%, 80%, 85%, 90%, 95%, or 100% of the binding affinity of the reference antibody or antigen-binding portion, or may exceed the binding affinity of the reference antibody or antigen-binding portion. In some embodiments, a variant antibody or an antigen-binding portion thereof may have mutations, e.g., that increase its half-life, alter its immunogenicity, provide a site for covalent or non-covalent binding to another molecule, etc. In certain embodiments, the variant antibody or antigen-binding portion thereof may have mutations in its FRs (e.g., in one, two, three, four, five, six, seven, or eight of its FRs). In certain embodiments, the variant antibody or antigen-binding portion thereof may have mutations in its CDRs (e.g., in one, two, three, four, five, or six of its CDRs). In certain embodiments, the variant antibody or antigen-binding portion thereof may have mutations in its constant regions.

The class of an anti-ROR2 antibody described herein may be changed or switched with another class or subclass. For example, an anti-ROR2 antibody that was originally IgM may be class switched to IgG. Further, the class switching may be used to convert one IgG subclass to another, e.g., from IgG₁ to IgG₂. A κ light chain constant region can be changed, e.g., to a λ light chain constant region, or vice versa.

The anti-ROR2 antibody of the present disclosure can be an IgG, an IgM, an IgE, an IgA, or an IgD molecule, but is typically of the IgG isotype, e.g., of IgG subclass IgG₁, IgG₂, IgG₃ or IgG₄. In some embodiments, the anti-ROR2 antibody is of human IgG₁ isotype subtype.

In some embodiments, the anti-ROR2 antibody may comprise at least one mutation in the Fc region. A number of different Fc mutations are known, where these mutations alter, e.g., the antibody's effector functions or half-life. For example, in some embodiments, the anti-ROR2 antibody comprises at least one mutation in the Fc region that reduces or eliminates effector function. In certain embodiments, the anti-ROR2 antibody may comprise, e.g., L234A, L235A, and/or P329A mutations (Eu numbering), wherein the mutations may appear alone or in any combination. In particular embodiments, the anti-ROR2 antibody may comprise an Fc region with all three mutations.

### II. Anti-ROR2 Antibody-Radionuclide Conjugates

An antibody-radionuclide conjugate (ARC) of the present disclosure comprises an anti-ROR2 antibody or an antigen-binding portion thereof conjugated to one or more radioactive isotopes, for the purposes of diagnostic, palliative, or therapeutic nuclear medicine. Such radioactive isotopes may include metal radionuclides (e.g., zirconium, lutetium, terbium, lead, and actinium) or halogen radionuclides (e.g., fluorine, bromine, iodine, and astatine). In some cases, the radioactive isotope can be directly labeled onto the anti-ROR2 antibody or antigen-binding portion thereof. In other disclosures, the radioactive isotope can be labeled to the anti-ROR2 antibody or antigen-binding portion thereof through use of a radiolabeling prosthetic linker, such as a metal chelation linker or a halogenation linker. Many radioactive isotopes that can serve as a cytotoxic or diagnostic moiety in an immunoconjugate are independently too toxic to be used for cancer treatment and thus are more effective and safer when specifically targeted to the cancer cell by an antibody or antigen-binding portion thereof.

For diagnostic imaging, an ARC of the present disclosure may be introduced (e.g., through parenteral injection such as intravenous injection) to the subject (e.g., a human or veterinary patient) at a diagnostically effective amount, followed by scanning the body with a PET detector, a SPECT detector, or a scintillation detector to generate images depicting the target-expressing cells (e.g., tumor) in the body of the subject. Such diagnostic images may be used for identifying the presence of ROR2-expressing disease, quantifying ROR2 ARC uptake in cancerous and normal tissue, stratifying subjects based on heterogeneity and intensity of ROR2 expression, and tracking ROR2 expression in subjects in response to treatments.

For radiotherapy, an ARC of the present disclosure may be introduced (e.g., through parenteral injection such as intravenous injection) to the subject (e.g., a human or veterinary patient) at a therapeutically effective amount, whereby the progress of the cancer (e.g., tumor growth and metastasis) is inhibited, leading to, e.g., progression-free survival, tumor shrinkage, tumor regression, and increased overall patient survival.

Depending on the purpose, the radioactive isotope may be selected on the basis of its medically advantageous radioactive emissions, either individually or in combination, e.g. gamma photons, x-ray photons, bremsstrahlung photons, positrons, internally converted electrons, Auger electrons, beta particles, and alpha particles. For PET imaging, the group of radioactive isotopes can consist of ¹¹C, ¹³N, ¹⁸F, ⁶⁴Cu, ⁶⁸Ga, ⁷⁸Br, ⁸³Sr, ⁸⁶Y, ⁸⁹Zr, ¹²⁴I, ¹⁵²Tb, and ²⁰³Pb. For SPECT imaging, the group of radioactive isotopes may consist of ^{99m}Tc, ¹¹¹In, ¹²³I, ¹³¹I, ¹³³Xe, ¹⁵⁵Tb, ¹⁷⁷Lu, and ²⁰¹Tl. For targeted beta particle therapy, the radioactive isotopes may be selected from ³²P, ³³P ⁶⁷Cu, ⁸⁹Sr, ⁹⁰Y ¹⁰⁵Rh, ¹³¹J ¹⁵³Sm, ¹⁶¹Tb, ¹⁶⁵Dy ¹⁶⁶Ho, ¹⁷⁷Lu, ¹⁸⁶Rh, and ¹⁹⁹Au. For targeted alpha particle therapy, the radioactive isotopes may be selected from ¹⁴⁹Tb, ²¹¹At, ²¹²Pb, ²¹²Bi, ²¹³Bi, ²²³Ra, ²²⁴Ra, ²²⁵Ac, and ²²⁷Th. In some embodiments, an ARC of the present disclosure is generated in a multi-step radiolabeling reaction by first radiolabeling the isotope to a prosthetic linker, such as a bifunctional chelate linker (or an active ester thereof) a halogenation precursor (or protected form of the precursor), or a chemical precursor and the labeled prosthetic is then purified. The radiolabeled prosthetic is then sequentially conjugated to a solvent-accessible site on a biomolecule (a ROR2 binder herein). Typically, biomolecule conjugation occurs at either a lysine (Lys or K) or cysteine (Cys or C) residue.

In some embodiments, an ARC of the present disclosure is generated in a 1-step radiolabeling reaction whereby the radioactive isotope is chemically labeled to the prosthetic group that has been pre-conjugated to the biomolecule (a ROR2 binder herein). Typically, the biomolecule conjugation occurs at either a lysine (Lys or K) or cysteine (Cys or C) residue. The radiolabeled biomolecule is then purified.

In some embodiments, the ARC is generated by conjugating a radionuclide to one or more lysine residues in the ROR2 binder, optionally via a linker. Lysine residues are abundant in antibodies, providing numerous potential sites for radionuclide attachment. Conjugation at lysine residues forms a stable bond that securely anchors the radionuclide to the antibody as it circulates through the bloodstream en route to the target tissue. This stability significantly reduces the risk of radionuclide detachment, minimizing off-target toxicity and ensuring precise delivery of the radionuclide to the intended site, thereby enhancing overall efficacy and safety.

A ROR2 binder of the present disclosure can be radiolabeled with more than one radioactive isotope atom (e.g., 1, 2, 3, 4, 5, 6, 7, 8, 9, or 10). The radiolabeling may be achieved through use of radiolabeled prosthetic compounds, radiometal chelation linkers or active ester thereof, radiohalogen precursors, or direct radiolabeling of constituent amino acids. In some embodiments, the number of radioisotope atoms conjugated to each antibody or antigen-binding portion of an ARC may range from 0 to 10, such as 0, 1, 2, 3, 4, 5, 6, 7, 8, 9, or 10. In some embodiments, the average number of radioactive isotopes labeled to the bulk antibody (the average drug-to-antibody ratio or DAR in an ARC composition) may range from 0 to 2, e.g., 0, 1, 2, or an intermediate value between adjacent integers. For example, the DAR of an ARC composition may be 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. A desired DAR may be achieved by controlling the reaction conditions (e.g., reactant concentrations, isotope activity, pH, reaction time, temperature, form of the radioactive isotope, solvent of the radioactive isotope, solvent of the reaction, and the presence of catalysts) and the post-reaction conditions (e.g., Sephadex™ size exclusion, mobile phase, and elution buffer excipients).

A ROR2 binder of the present disclosure can be conjugated to one or more radiolabeling prosthetic linker to improve radiolabeling efficiency, raise ARC specific activity, and improve chemical manufacturing performance for radiolabeling process scalability. The conjugation may be made through reactive chemical moieties, bifunctional chelators or active ester thereof, halogenation prosthetic linkers and precursors thereof. In some embodiments, the average number of conjugated prosthetic linkers (linker-to-antibody ratio, or LAR) can range from 0 to 10, such as 0, 1, 2, 3, 4, 5, 6, 7, 8, 9, or 10, or an intermediate value within this range. For example, the LAR of an ARC composition is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. In some embodiments, the ARC has a LAR of <1. In some embodiments, the ARC composition herein has a LAR of 3 to 5, e.g., 3.2 to 4.8, 3.2 to 3.8, or 3.5 to 4.5. In further embodiments, the ARC composition herein a LAR of about 4. A desired LAR may be achieved by controlling reaction stoichiometry conditions (e.g., reactants concentration, reaction time, pH, and solvents) when making ARCs and post-reaction purification (e.g., Sephadex™ size exclusion, mobile phase, and elution excipients).

A labeled prosthetic compound/radical or a radiohalogen precursor (alone or attached to a macromolecule such as a ROR2 binder herein) may generally include, in addition to a radioactive isotope, a charged group (CG), and a macromolecule conjugating moiety (MMCM). In some embodiments, an ARC comprises a ROR2 binder (targeting moiety), a radiolabeled prosthetic group or precursor, and optionally, a chelating agent (e.g., macrocyclic or acyclic).

The ARCs herein may be prepared by a method that enhances the retention of a radionuclide in targeted ROR2-positive cells, such as ROR2-positive cancer cells. For example, the preparation method uses labeling techniques that allow generation of a charged catabolite following intracellular proteolysis, which cannot traverse the lysosomal or cell membrane and is resistant to exocytosis, such that the portion of the ARC bearing the radioactive label is inert to lysosomal degradation and becomes trapped inside the cell after proteolysis. Exemplary synthesis routes for making the present ARCs are described in the Working Examples.

### A. ARCs for PET-Based Diagnosis

The ARCs in the present disclosure may be used for PET imaging to identify and locate ROR2-expressing cancer in a patient. PET is a sensitive imaging technology with excellent quantitative capability. PET imaging may be used to diagnose cancer, to determine the appropriateness of treating a cancer with anti-ROR2 therapeutics, and to monitor the progress of cancer treatment. In the structural formulae herein, “Ab” denotes the anti-ROR2 antibody or an antigen-binding portion thereof. In some embodiments, the Ab is Ab1. In some embodiments, the Ab is Ab2. In some embodiments, the Ab is Ab3. In some embodiments, the Ab is Ab4.

### 1. Fluorine-18

In some embodiments, the ARC comprises fluorine-18 (¹⁸F), which has a half-life of 109.7 minutes. Methods of labeling a biomolecule, such as an ROR2 binder, with fluorine-18 are disclosed in, e.g., U.S. Pat. No. 9,839,704.

### 2. Zirconium-89

In some embodiments, the ARC comprises zirconium-89 (⁸⁹Zr), which has a half-life of 78.41 hours. Zirconium-89 may be conjugated to a biomolecule such as a ROR2 binder herein by, e.g., a metal chelation linker.

In some embodiments, an acyclic chelation approach may be taken using p-SCN-benzyl-Deferoxamine (p-SCN-Bn-DFO; Formula I below).

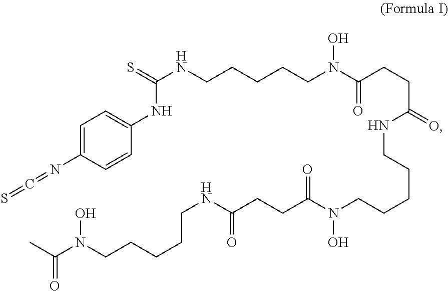 [SMILES: CC(=O)N(O)CCCCCNC(=O)CCC(=O)N(O)CCCCCNC(=O)CCC(=O)N(O)CCCCCNC(=S)Nc1ccc(N=C=S)cc1]

with an exemplary ARC shown below (LAR is not illustrated below; i.e., each Ab may be conjugated to a ⁸⁹Zr prosthetic moiety at more than one lysine residue):

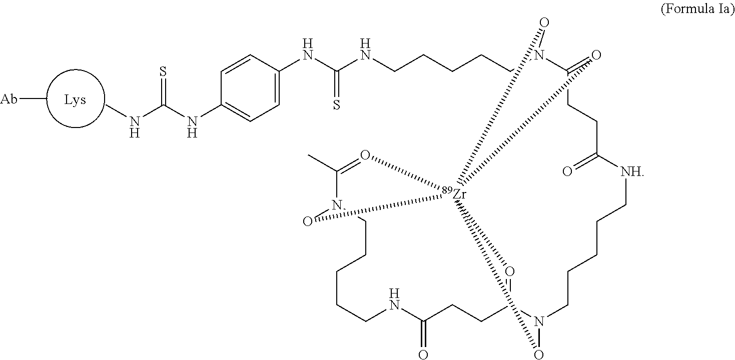

In some embodiments, an acyclic chelation approach may be taken using Fe-DFO-N-suc-TFP; (Formula II below).

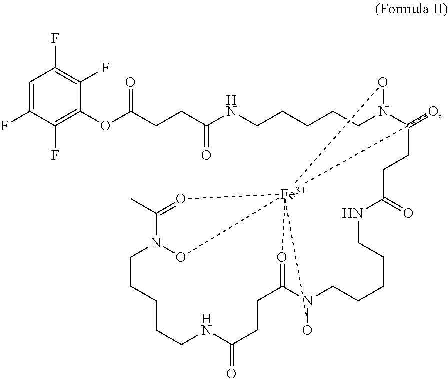

with an exemplary ARC shown below (LAR is not illustrated below; i.e., each Ab may be conjugated to a ⁸⁹Zr prosthetic moiety at more than one lysine residue):

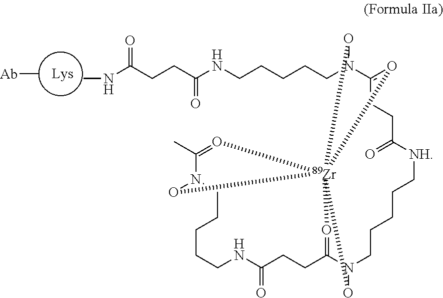

In some embodiments, an acyclic chelation approach may be taken using DFO-O3 (p-SCN-Bn-DFO-O3, Formula III).

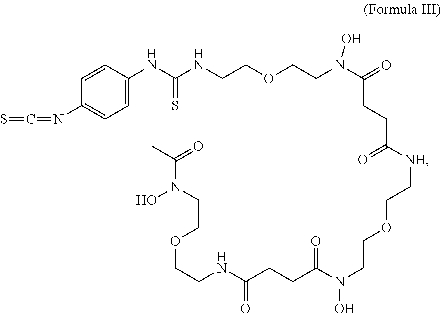 [SMILES: CC(=O)N(O)CCOCCNC(=O)CCC(=O)N(O)CCOCCNC(=O)CCC(=O)N(O)CCOCCNC(=S)Nc1ccc(N=C=S)cc1]

with an exemplary ARC shown below (LAR is not illustrated below; i.e., each Ab may be conjugated to a ⁸⁹Zr prosthetic moiety at more than one lysine residue):

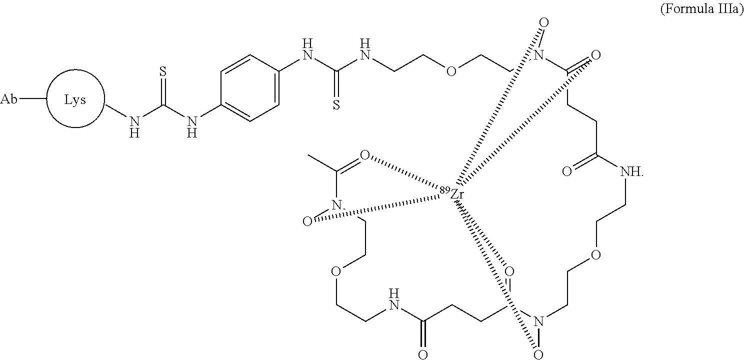

In some embodiments, an acyclic chelation approach may be taken using DFO-Mal (Mal-Bn-DFO, Formula IV).

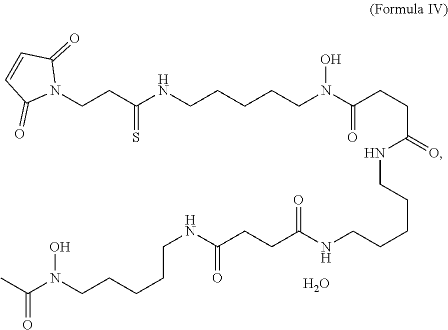

with an exemplary ARC shown below (LAR is not illustrated below; i.e., each Ab may be conjugated to a ⁸⁹Zr prosthetic moiety at more than one cysteine residue):

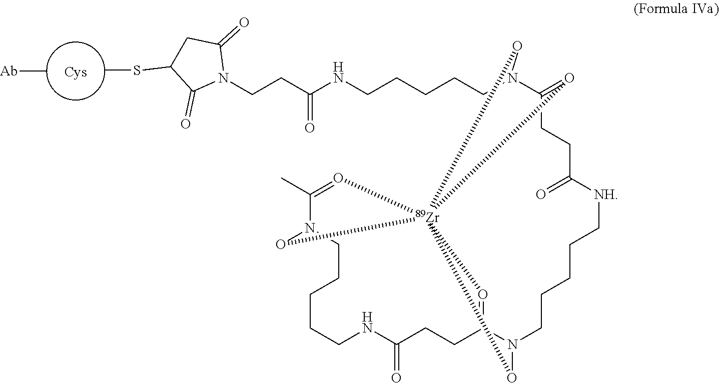

In some embodiments, an acyclic chelation approach may be taken using DFO-Km (p-SCN-Ben-DFO-Km, Formula V).

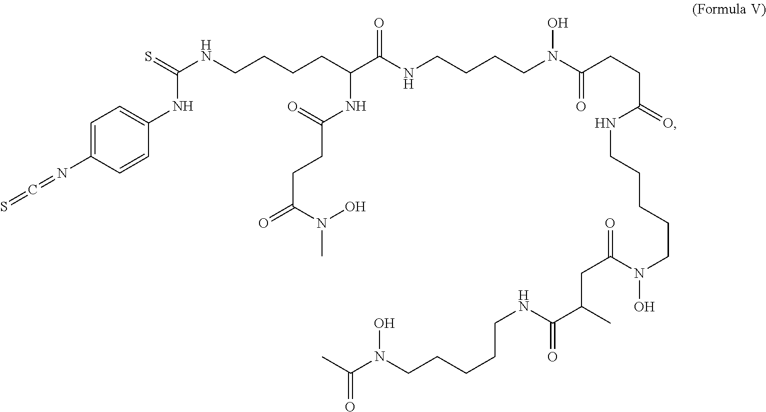 [SMILES: CC(=O)N(O)CCCCCNC(=O)C(C)CC(=O)N(O)CCCCCNC(=O)CCC(=O)N(O)CCCCNC(=O)C(CCCCNC(=S)Nc1ccc(N=C=S)cc1)NC(=O)CCC(=O)N(C)O]

with an exemplary ARC shown below (LAR is not illustrated below; i.e., each Ab may be conjugated to a ⁸⁹Zr prosthetic moiety at more than one lysine residue):

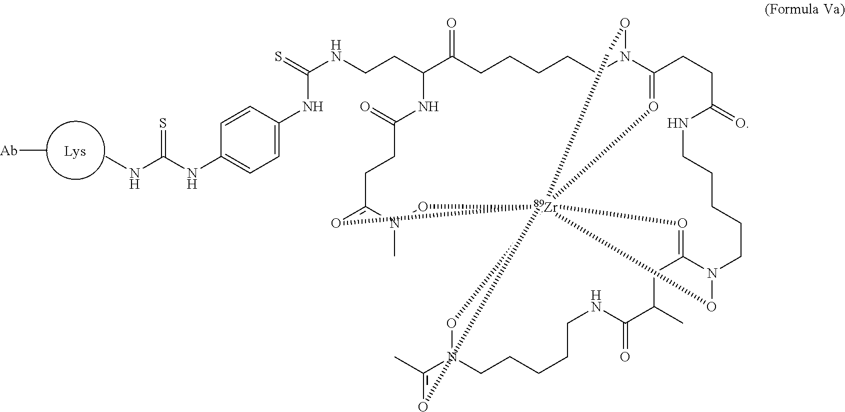

In some embodiments, an acyclic chelation approach may be taken using DFO* (p-SCN-Ben-DFO* Formula VI).

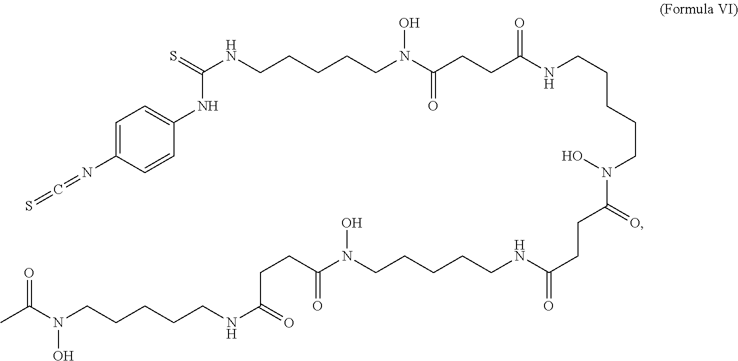 [SMILES: CC(=O)N(O)CCCCCNC(=O)CCC(=O)N(O)CCCCCNC(=O)CCC(=O)N(O)CCCCCNC(=O)CCC(=O)N(O)CCCCCNC(=S)Nc1ccc(N=C=S)cc1]

with an exemplary ARC shown below (LAR is not illustrated below; i.e., each Ab may be conjugated to a ⁸⁹Zr prosthetic moiety at more than one lysine residue):

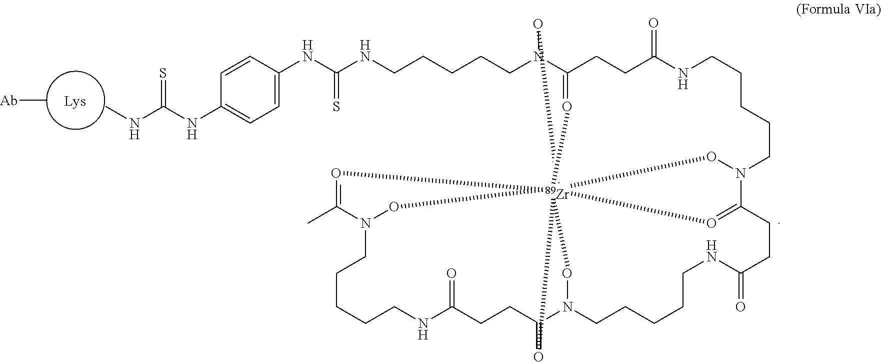

In some embodiments, an acyclic chelation approach may be taken using DFO2 SCN-Ben-DFO2 Formula VII).

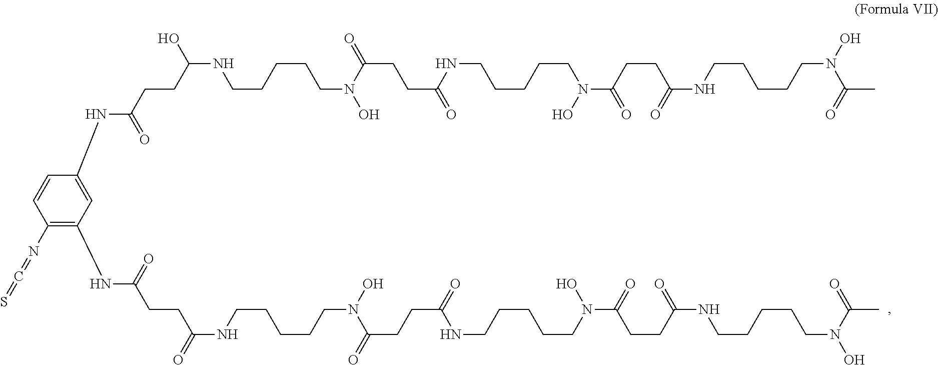 [SMILES: CC(=O)N(O)CCCCCNC(=O)CCC(=O)N(O)CCCCCNC(=O)CCC(=O)N(O)CCCCCNC(=O)CCC(=O)Nc1cc(NC(=O)CCC(O)NCCCCCN(O)C(=O)CCC(=O)NCCCCCN(O)C(=O)CCC(=O)NCCCCCN(O)C(C)=O)ccc1N=C=S]

with an exemplary ARC shown below (LAR is not illustrated below; i.e., each Ab may be conjugated to a ⁸⁹Zr prosthetic moiety at more than one lysine residue):

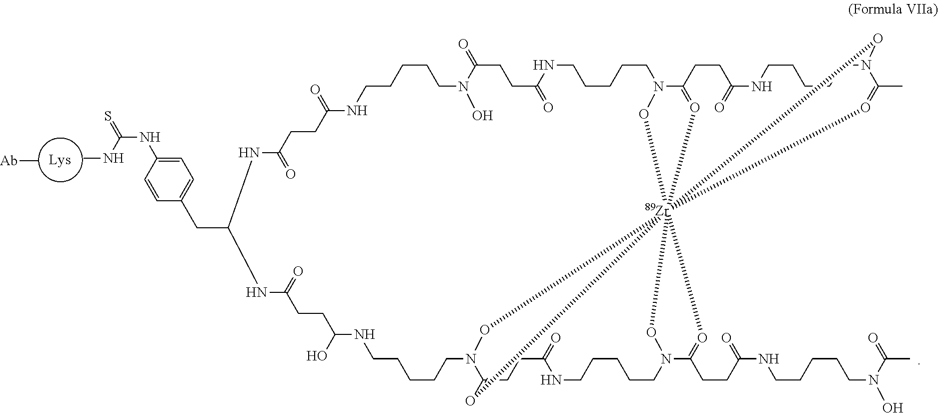

In some embodiments, an acyclic chelation approach may be taken using p-SCN-Bn-HOPO (see, e.g., Deri et al., Bioconjugate Chem. (2015) 26(12):2579-91) (Formula VIII below).

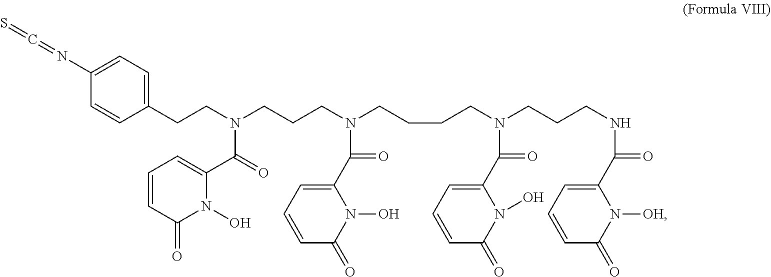 [SMILES: O=C(NCCCN(CCCCN(CCCN(CCc1ccc(N=C=S)cc1)C(=O)c1cccc(=O)n1O)C(=O)c1cccc(=O)n1O)C(=O)c1cccc(=O)n1O)c1cccc(=O)n1O]

with an exemplary ARC shown below (LAR is not illustrated below; i.e., each Ab may be conjugated to a ⁸⁹Zr prosthetic moiety at more than one lysine residue):

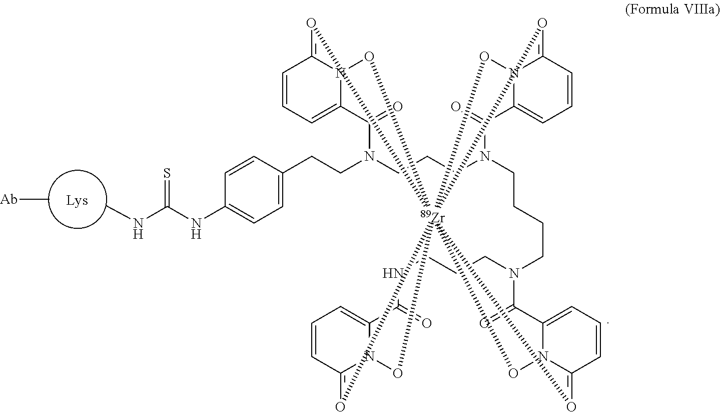

In some embodiments, a cyclic chelation approach may be taken using DOTA (dodecane tetraacetic acid, Formula IX), an active ester thereof, or DOTA with a hydrophilic spacer (e.g. PEG) extending the distance between the chelate and the macromolecular conjugation domain.

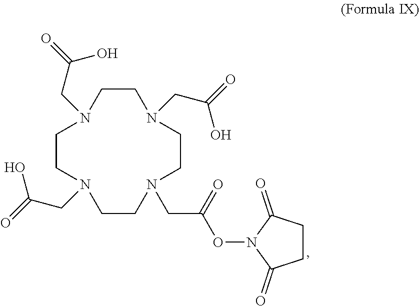 [SMILES: O=C(O)CN1CCN(CC(=O)O)CCN(CC(=O)ON2C(=O)CCC2=O)CCN(CC(=O)O)CC1]

with an exemplary ARC shown below (LAR is not illustrated below; i.e., each Ab may be conjugated to a ⁸⁹Zr prosthetic moiety at more than one lysine residue):

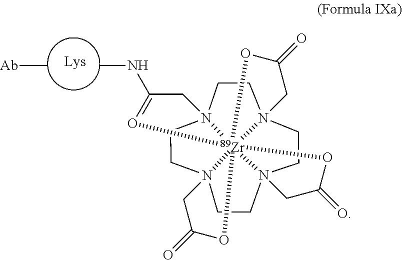

In some embodiments, a cyclic chelation approach may be taken using DOTA-GA-nhs (Formula X), an active ester thereof, or DOTA-GA with a hydrophilic spacer (e.g. PEG) extending the distance between the DOTAGA to the chelate and the macromolecular conjugation domain.

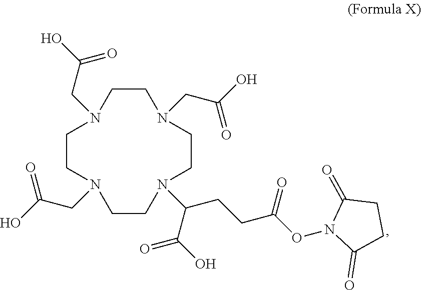 [SMILES: O=C(O)CN1CCN(CC(=O)O)CCN(C(CCC(=O)ON2C(=O)CCC2=O)C(=O)O)CCN(CC(=O)O)CC1]

with an exemplary ARC shown below (LAR is not illustrated below; i.e., each Ab may be conjugated to a ⁸⁹Zr prosthetic moiety at more than one lysine residue):

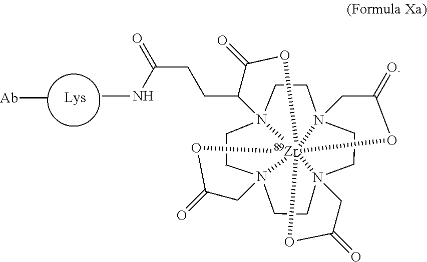

In some embodiments, a cyclic chelation approach may be taken using Macropa-SCN (Formula XI), an active ester thereof, or Macropa with a hydrophilic spacer (e.g., PEG) extending the distance between the chelate and the macromolecular conjugation domain.

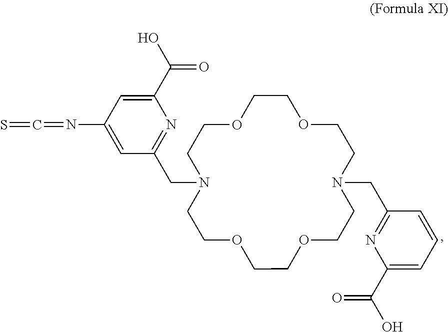 [SMILES: O=C(O)c1cccc(CN2CCOCCOCCN(Cc3cc(N=C=S)cc(C(=O)O)n3)CCOCCOCC2)n1]

with an exemplary ARC shown below (LAR is not illustrated below; i.e., each Ab may be conjugated to a ⁸⁹Zr prosthetic moiety at more than one lysine residue):

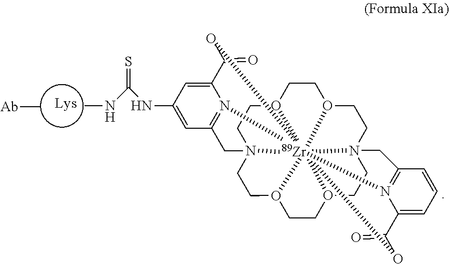

### 3. Iodine-124

In some embodiments, the ARC comprises iodine-124, which has a half-life of 4.18 days. Iodine-124 may be conjugated to a biomolecule such as an ROR2 binder herein by, e.g., a halogenation linker, such as the N-succinimidyl-4-guanidinomethyl-3-[*I]iodobenzoate (iso-SGMIB) linker system described in Choi et al., Nucl Med Biol. (2014) 41(10):802-12 and WO 2018/178936. An exemplary [¹²⁴I]-iso-SGMIB ARC is shown below (LAR is not illustrated below; i.e., each Ab may be conjugated to an ¹²⁴I prosthetic moiety at more than one lysine residue):

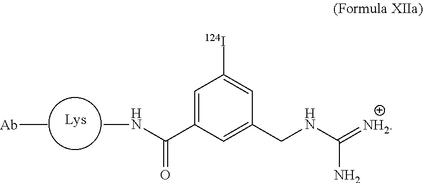

### 4. Copper-64

In some embodiments, the ARC comprises copper-64 (⁶⁴Cu), which has a half-life of 12.7 hours. Copper-64 may be conjugated to a biomolecule such as a ROR2 binder herein by use of a cyclic chelator, such as DOTA, DOTAGA, or a sarcophagine chelator (e.g., DiamSar, SarAr, AnAnSar, BaMalSar, and Mal2Sar). See Cai et al, J Labelled Comp Radiopharm. (2014) 57(4):224-230 and Liu et al., J Nuc Medicine. (2012) 53 (supplement1); 1525).

### B. ARCs for Alpha-Particle Therapy

The ARCs in the present disclosure may be used in alpha-particle therapy to treat cancer (e.g., solid tumors). Targeted alpha-particle therapy (TAT) using ARCs selectively deliver radionuclides emitting alpha-particles (cytotoxic payload) to tumors. Due to the high linear energy transfer (LET) and short range of alpha particles in tissue, the alpha-particle ARCs can effectively kill cancer cells while causing minimal toxicity to surrounding healthy tissue (see, e.g., Tafreshi et al., Molecules (2019) 24:4314). Examples of alpha-emitting radionuclides are ²²⁵Ac, ²¹¹At, ²¹²Bi, ²¹³Bi, ²¹²Pb, ²²³Ra, ²²⁴Ra, ¹⁴⁹Tb and ²²⁷Th. The physical properties of these radionuclides are shown in Table A below (Tafreshi, supra).

TABLE A
Exemplary Alpha-Emitting Radioisotopes

| | | |
|---|---|---|
| | | Max |
| Isotope | Half-Life | Energy (MeV) |

| | | | |
|---|---|---|---|
| ²²⁵Ac | 10.1 | d | 5.83 |
| ²¹¹At | 7.2 | h | 5.87 |
| ²¹²Bi | 1.01 | h | 6.09 |
| ²¹³Bi | 45.6 | min | 5.87 |
| ²¹²Pb | 10.6 | h | 6.09 |
| ²²³Ra | 11.4 | d | 5.87 |
| ²²⁴Ra | 3.6 | d | 8.8 |
| ¹⁴⁹Tb | 4.1 | h | 3.96 |
| ²²⁷Th | 18.7 | d | 6.04 |

In some embodiments, the ARC comprises astatine-211 (²¹¹At), which has a half-life of 7.2 hours. Astatine-211 may be conjugated to a biomolecule such as a ROR2 binder herein by, e.g., a halogenation linker, such as the N-succinimidyl-3-[²¹¹At]astato-5-guanidinomethyl benzoate (iso-SAGMB or iso-[²¹¹At]SAGMB) linker system described in Vaidyanathan et al., Nucl Med Biol. (2003) 30:351-9, Choi et al., Nucl Med Biol. (2017) 56:10-20, and WO 2018/178936. An exemplary [²¹¹At]-iso-SAGMB ARC is shown below (LAR not illustrated):

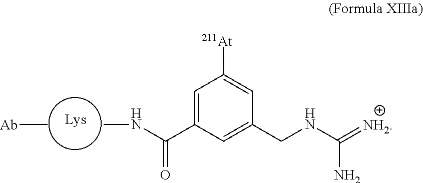

Additional examples of alpha-emitting ARCs are shown below (LAR not illustrated):

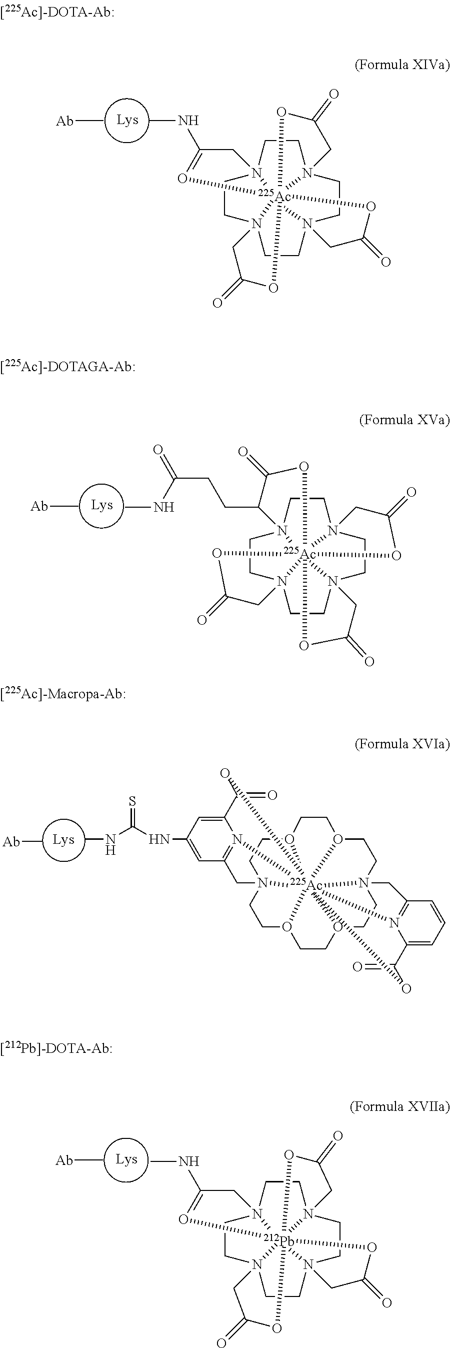

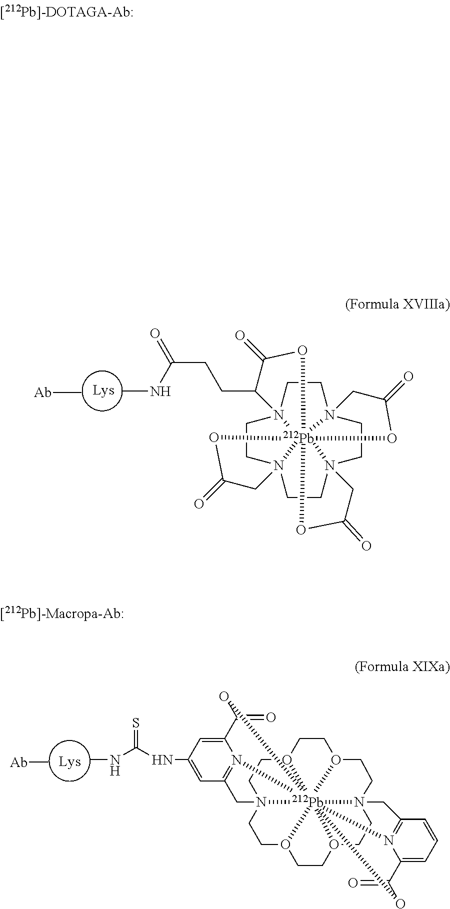

### C. ARCs for Beta-Particle Therapy

The ARCs in the present disclosure may be used in beta-particle therapy to treat cancer (e.g., solid tumors). Beta-particles are negatively charged electrons emitted from the nucleus of decaying radioactive atoms (one electron/decay). Because of their small mass, the recoil energy of the daughter nucleus is negligible and the LET of these energetic and negatively (−1) charged particles is very low (~0.2 keV/μm) along their path, which is up to 1 centimeter. As a result, their therapeutic efficacy is based on the presence of very high radionuclide concentrations within the targeted tissue (Kassis, Semin Nucl Med. (2008) 38(5):358-66).

The properties of exemplary beta-emitters are shown in Table B below (Kassis, supra):

TABLE B
Exemplary Beta-Emitting Radioisotopes

| | | | E_{β}⁻(max) | R_{β}⁻(max) |
|---|---|---|---|---|
| Radionuclide | Half-Life | | (keV)^{I} | (mm)^{H} |
| ³³P | 25.4 | d | 249 | 0.63 |
| ¹⁷⁷Lu | 6.7 | d | 497 | 1.8 |
| ⁶⁷Cu | 61.9 | h | 575 | 2.1 |
| ¹⁶¹Tb | 6.9 | d | 593 | 2.2 |
| ¹³¹T | 8.0 | d | 606 | 2.3 |
| ¹⁸⁶Re | 3.8 | d | 1,077 | 4.8 |
| ¹⁶⁵Dy | 2.3 | h | 1,285 | 5.9 |
| ⁸⁹Sr | 50.5 | d | 1,491 | 7.0 |
| ³²P | 14.3 | d | 1,710 | 8.2 |
| ¹⁶⁶Ho | 28.8 | h | 1,854 | 9.0 |
| ¹⁸⁸Re | 17.0 | h | 2,120 | 10.4 |
| ⁹⁰Y | 64.1 | h | 2,284 | 11.3 |

In some embodiments, the ARC comprises iodine-131 (¹³¹I), which has a half-life of 8 days. Iodine-131 may be conjugated to a biomolecule such as a ROR2 binder herein by, e.g., a halogenation linker, such as the iso-SGMIB linker system described above. An exemplary [¹³¹I]-iso-SGMIB ARC is shown below (LAR not illustrated):

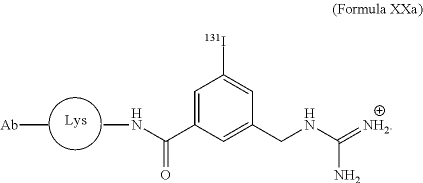

Additional examples of beta-emitting ARCs are shown below (LAR not illustrated):

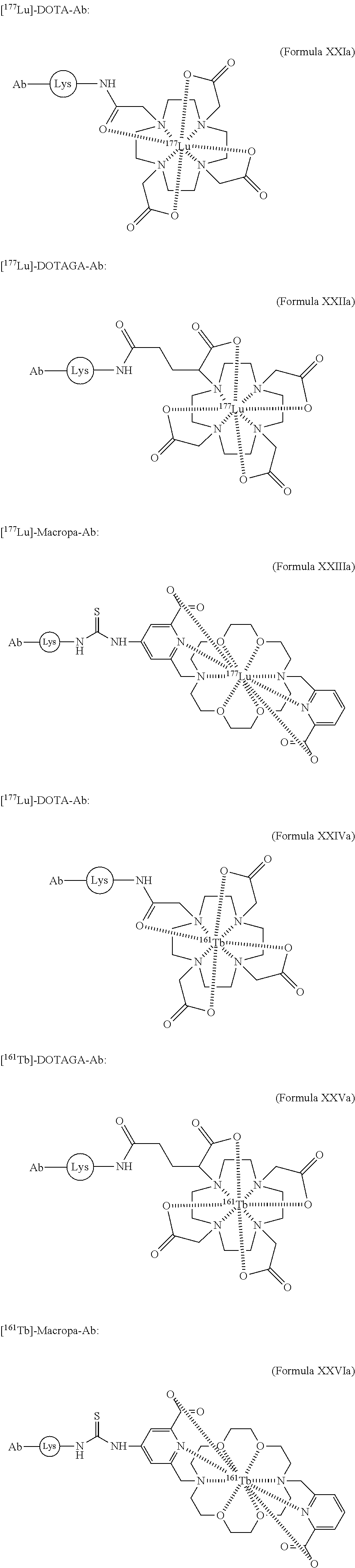

In some embodiments, the ARC comprises copper-67 (⁶⁷Cu), which has a half-life of 2.58 days. Copper-67 may be conjugated to a biomolecule such as a ROR2 binder herein by use of a cyclic chelator, such as DOTA, DOTAGA, or a sarcophagine chelator (e.g., DiamSar, SarAr, AnAnSar, BaMalSar, and Mal2Sar). See Cai et al, J Labelled Comp Radiopharm. (2014) 57(4):224-230; and Liu et al., J Nuc Medicine. (2012) 53 (supplement1); 1525).

### D. ARCs for Auger Electron Therapy

The ARCs in the present disclosure may be used in Auger electron therapy to treat cancer (e.g., solid tumors). During the decay of many radioactive atoms, a vacancy is formed (usually in the K shell) as a consequence of electron capture and/or internal conversion. These vacancies are quickly filled by an electron from a higher shell. This process leads to a cascade of atomic electron transitions that move the vacancy toward the outermost shell. The inner-shell electron transitions result in the emission of characteristic X-ray photons or an Auger, Coster-Kronig, or super Coster-Kronig monoenergetic electron (collectively called Auger electrons). See Kassis, supra. An average of 5 to 30 Auger electrons, with energies ranging from a few eV to approximately 1 keV, are emitted per decaying atom. The very low energies of Auger electrons necessitate their close proximity to the radiosensitive target (e.g., DNA) for radiotherapeutic effectiveness. The properties of exemplary Auger electron emitters are shown in Table C below (Kassis, supra):

TABLE C
Exemplary Auger Electron Emitting Radioisotopes
Total electron yield per decay

| Radionuclide | | | “Long” range | “Short” range | “Very short” range |
|---|---|---|---|---|---|
| (# *) | Half-Life | | electrons (%) | electrons (%) | electrons (%) |
| ¹²⁵I (20) | 60.5 | d | 20 (98%) | 18 (86%) | 8 (39%) |
| ¹²³I (11) | 13.3 | h | 11 (98%) | 10 (89%) | 5 (40%) |
| ⁷⁷Br (7) | 57 | h | 7 (100%) | 6 (95%) | 3 (51%) |
| ¹¹¹In (15) | 3 | d | 15 (98%) | 14 (91%) | 8 (53%) |
| ^{195m}Pt (36) | 4 | d | 33 (92%) | 33 (79%) | 7 (19%) |

| | | | |
|---|---|---|---|
| Range: | <0.5 μm | <100 nm | <2 nm |
| LET^{H} | 4-26 | 9-26 | <18 |

*Average number of electrons emitted/decay.

In some embodiments, the ARC comprises iodine-125 (¹²⁵I), which has a half-life of 60.5 days. Iodine-125 may be conjugated to a biomolecule such as an ROR2 binder herein by, e.g., a halogenation linker, such as the iso-SGMIB linker system described above.

An example of [¹²⁵I]-iso-SGMIB ARC is shown below (LAR & DAR not illustrated):

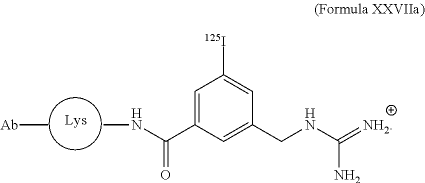

### E. Bifunctional ARCs for Theranostic Applications

The ARCs disclosed herein may be conjugated to a cytotoxic drug to develop dual-action, antibody-based therapeutics, providing a combination of 1) precise radioimaging or radiation therapy and 2) targeted cytotoxicity, thereby synergistically enhancing treatment precision and therapeutic efficacy. Such bifunctional ARCs can be generated by attaching a cytotoxic payload to the ARC, optionally through a linker, which may be cleavable or non-cleavable. In some embodiments, the radionuclide and cytotoxic payload are conjugated at distinct sites on the antibody or antibody fragment. In particular embodiments, the radionuclide is conjugated to lysine residues through a linker, while the cytotoxic drug is conjugated to cysteine residues via a separate linker, ensuring clear functional separation and facilitating efficient payload delivery.

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab is an anti-ROR2 antibody described herein and the antibody is of human IgG₁ isotype subtype.

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab comprises the HCDRs and LCDRs of Ab1. In further embodiment, the Ab comprises the V_{H} and V_{L} of Ab1. In certain embodiments, the Ab comprises the heavy chain and light chain of Ab1.

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab comprises the HCDRs and LCDRs of Ab2. In further embodiment, the Ab comprises the V_{H} and V_{L} of Ab2. In certain embodiments, the Ab comprises the heavy chain and light chain of Ab2.

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab comprises the HCDRs and LCDRs of Ab3. In further embodiment, the Ab comprises the V_{H} and V_{L} of Ab3. In certain embodiments, the Ab comprises the heavy chain and light chain of Ab3.

In some embodiments, an immunoconjugate of the present disclosure is represented by any one of Formula Ia, Formula IIa, Formula IIIa, Formula IVa, Formula Va, Formula VIa, Formula VIIa, Formula VIIIa, Formula IXa, Formula Xa, Formula XIa, Formula XIIa, Formula XIIIa, Formula XIVa, Formula XVa, Formula XVIa, Formula XVIIa, Formula XVIIIa, Formula XIXa, Formula XXa, Formula XXIa, Formula XXIIa, Formula XXIIIa, Formula XXIVa, Formula XXVa, Formula XXVIa, or Formula XXVIIa, wherein the Ab comprises the HCDRs and LCDRs of Ab4. In further embodiment, the Ab comprises the V_{H} and V_{L} of Ab4. In certain embodiments, the Ab comprises the heavy chain and light chain of Ab4.

### III. Pharmaceutical Compositions

Another aspect of the present disclosure is a pharmaceutical composition comprising as an active ingredient (or as the sole active ingredient) a ROR2 ARC as described herein. The pharmaceutical composition may additionally comprise a pharmaceutically acceptable excipient. “Pharmaceutically acceptable excipients” may include appropriate solvents, dispersion media, antibacterial and antifungal agents, isotonic agents, and the like. Examples of pharmaceutically acceptable excipients are water and saline (e.g., phosphate-buffered saline).

The pharmaceutical compositions herein may be used to treat cancer, e.g., ROR2-expressing cancer.

A pharmaceutical composition of the present disclosure may comprise a therapeutically or diagnostically effective amount of an ARC described herein. A “therapeutically effective amount” is an amount of the drug (e.g., an ARC described herein) or a pharmaceutical composition comprising it that will relieve to some extent one or more of the symptoms of the disease being treated. A therapeutically effective amount of an anti-cancer therapeutic may, for example, result in delayed tumor growth, elimination of cancer cells, tumor shrinkage, increased survival, slowed or decreased metastasis, or other clinical endpoints desired by healthcare professionals. In some embodiments, the ARC composition to be administered to the patient has radioactivity of less than 200 mCi. In some embodiments, the ARC composition to be administered to the patient has radioactivity of less than 30 mCi. In some embodiments, the ARC composition to be administered to the patient has radioactivity of less than 10 mCi. In some embodiments, the ARC composition to be administered to the patient has radioactivity of 5 mCi or less. In some embodiments, the ARC composition to be administered to the patient has radioactivity of 1 mCi or less. In some embodiments, the ARC composition to be administered to the patient has radioactivity of 500 μCi or less. In some embodiments, the ARC composition to be administered to the patient has radioactivity of 100 μCi or less. In some embodiments, the ARC composition to be administered to the patient has radioactivity of about 50 μCi.

A “diagnostically effective amount” is an amount of the drug that is sufficient for optimal imaging signal over background noise (e.g. blood pool, or liver activity levels) for the target tissue and is well below safety thresholds associated for a therapeutic dose. In some embodiments, the ARC diagnostic to be administered to the patient has radioactivity of less than 5 mCi. In some embodiments, the ARC diagnostic to be administered to the patient has radioactivity of less than 3 mCi. In some embodiments, the ARC diagnostic to be administered to the patient has radioactivity of less than 1 mCi.

In some embodiments, an ARC described herein may be co-administered or formulated with another medication/drug, e.g., for treatment of cancer. The additional medication/drug may comprise, e.g., a chemotherapeutic agent, an anti-neoplastic agent, or an anti-angiogenic agent.

The pharmaceutical compositions described herein may be delivered to a patient through parenteral administration. In particular, parenteral administration is contemplated to include, but is not limited to, subcutaneous, intraperitoneal, intramuscular, intrasternal, intracisternal, intravenous, intraarterial, intrathecal, intraurethral, intracranial, intratumoral, and intrasynovial injection or infusions. In some embodiments, the pharmaceutical composition is delivered intravenously (e.g., through intravenous infusion) or subcutaneously (e.g., through subcutaneous injection).

### IV. Clinical Uses

In some embodiments, the ROR2 ARCs of the present disclosure are used to diagnose or treat cancer in a patient (e.g., a mammal such as a human) in need thereof. In certain embodiments, the cancer is a ROR2-expressing cancer. The ARC may be administered alone or in combination with other therapeutic agents.

In some embodiments, the cancer treated by the ARCs of the present disclosure may be a solid tumor or a hematopoietic cancer. The cancer may be, e.g., melanoma, skin basal cell cancer, glioblastoma, glioma, gliosarcoma, astrocytoma, meningioma, neuroblastoma, adrenocortical cancer, head and neck cancer (e.g., cancer of the head, neck, nasal cavity, paranasal sinuses, nasopharynx, oral cavity, oropharynx, larynx, hypopharynx, and/or salivary glands, and paragangliomas), oral cancer, salivary gland cancer, nasopharyngeal cancer, breast cancer (e.g., triple negative breast cancer), lung cancer (e.g., non-small cell lung cancer (NSCLC), small cell lung cancer, or squamous cell lung cancer), esophageal cancer, gastroesophageal junction cancer, gastric cancer, gastrointestinal cancer, primary peritoneal cancer, liver cancer, hepatocellular carcinoma, gallbladder cancer, biliary tract cancer, cholangiocarcinoma, colon cancer, rectal cancer, colorectal carcinoma, ovarian cancer, fallopian tube cancer, bladder cancer, upper urinary tract cancer, urothelial cancer, renal cell carcinoma, kidney cancer, genitourinary cancer, cervical cancer, testicular cancer, prostate cancer, fibrosarcoma, liposarcoma, rhabdomyosarcoma (e.g., embryonal rhabdomyosarcoma), leiomyosarcoma, neurofibrosarcoma, synovial sarcoma, liposarcoma, alveolar soft part sarcoma, osteosarcoma, histiocytoma (e.g., malignant fibrous histiocytoma), pancreatic cancer, endometrial cancer, cancer of the appendix, thyroid cancer, advanced Merkel cell cancer, multiple myeloma, sarcomas, choriocarcinoma, leukemia (e.g., erythroleukemia, acute lymphoblastic leukemia, acute monocytic leukemia, acute promyelocytic leukemia, acute myeloid leukemia, acute myelogenous leukemia, chronic myeloid leukemia, chronic myelogenous leukemia, chronic lymphocytic leukemia, acute lymphoblastic leukemia, or mast cell leukemia), lymphoma (e.g., small lymphocytic lymphoma, Burkitt's lymphoma, Hodgkin's lymphoma, non-Hodgkin's lymphoma, diffuse large B cell lymphoma, lymphoplasmacytoid lymphoma, mucosa-associated lymphoid tissue lymphoma, mantle cell lymphoma, T-cell anaplastic large cell lymphoma, follicular lymphoma, monocytic lymphoma, or HTLV-associated T cell leukemia/lymphoma), or mesothelioma. In certain embodiments, the cancer is selected from the group consisting of head and neck cancer, bone cancer (e.g., osteosarcoma), Ewing sarcoma, squamous cell carcinoma, lung cancer (e.g., non-small cell lung cancer or small cell lung cancer), kidney cancer, urethral cancer, colorectal cancer, prostate cancer, glioblastoma multiforme, ovarian cancer, cervical cancer, pancreatic cancer, breast cancer (e.g., triple negative breast cancer), melanoma, liver cancer, bladder cancer, stomach cancer, esophageal cancer, and chronic myelogenous leukemia. In particular embodiments, the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma. The cancer may be, e.g., at an early, intermediate, late, locally advanced, or metastatic stage, and may be relapsed or refractory to other therapeutics, or there may be no standard therapy available.

In some embodiments, the ROR2 ARCs disclosed herein may serve as companion diagnostic tools for antibody drug conjugates (ADCs), leveraging their complementary abilities to provide highly specific radioimaging and potent target cytotoxicity. For example, in the context of patient selection, the ROR2 ARCs described herein can be used to visualize and quantify ROR2 expression levels before initiating treatment with ADCs, ensuring that only patients whose tumors express high levels of ROR2 are selected, thereby maximizing the likelihood of therapeutic efficacy while avoiding unnecessary exposure to the ADC.

The present ARCs also can be used in the context of biodistribution analysis. By assessing the pharmacokinetics and biodistribution of an ROR2 ARC in vivo, clinicians can gain insights into how the corresponding ADC behaves in the patient's body. This could help with identification of potential barriers, such as limited tumoral uptake, high off-target accumulation, or poor clearance profiles, which could affect ADC efficacy and/or safety.

The present ARCs also can be used in treatment monitoring. Since ARCs allow non-invasive imaging of tumor response throughout the course of treatment, clinicians can monitor therapeutic progress, tumor regression, or resistance to treatment by tracking the uptake and retention of the ARC in real time, enabling timely adjustments to the ADC therapy plan.

The present ARCs also can be used in dose optimization. ARCs provide quantitative imaging data that can inform ADC dosing strategies, ensuring an optimal balance between therapeutic efficacy and minimizing toxicity. For instance, imaging with AR_Cs can reveal whether a higher or lower dose of the ADC is required for effective tumor targeting.

The present ARCs can provide therapeutic synergy. For example, the ARCs themselves may provide dual diagnostic and therapeutic functions and the associated advantages. In cases where ADCs need additional support against resistant or residual disease, ARCs can provide localized radiation therapy in addition to guiding treatment planning through imaging.

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. In some embodiments, therapeutic use of an ARC described herein will result in delayed tumor growth, elimination of cancer cells, tumor shrinkage/regression, increased survival, slowed or decreased metastasis, or other clinical endpoints desired by healthcare professionals. In certain embodiments, therapeutic use of an ARC described herein inhibits tumor growth by at least 10, 20, 30, 40, 50, 60, 70, 80, 90, or 100%. In certain embodiments, therapeutic use of an ARC described herein provides partial tumor regression of at least 10, 20, 30, 40, 50, 60, 70, 80, or 90%, or complete tumor regression.

The ARCs of the present disclosure may be administered without additional therapeutic treatments, i.e., as a stand-alone therapy (monotherapy). Alternatively, treatment with the ARCs of the present disclosure may include at least one additional therapeutic treatment (combination therapy), e.g., an immunomodulatory agent, an anti-cancer agent (such as a chemotherapeutic agent, an anti-neoplastic agent, or an anti-angiogenic agent), a vaccine (such as a tumor vaccine), or radiation therapy.

In some embodiments, the additional therapeutic treatment may comprise an anti-cancer agent such as, for example, an agent selected from the group consisting of alkylating agents (e.g., platinum derivatives such as cisplatin, carboplatin and/or oxaliplatin); plant alkoids (e.g., paclitaxel, docetaxel and/or irinotecan); antitumor antibiotics (e.g., doxorubicin, adriamycin, daunorubicin, epirubicin, idarubicin mitoxantrone, dactinomycin, bleomycin, actinomycin, luteomycin, and/or mitomycin); topoisomerase inhibitors (e.g., topotecan); antimetabolites (e.g., fluorouracil and/or other fluoropyrimidines); kinase inhibitors such as tyrosine kinase inhibitors (e.g., acalabrutinib, ibrutinib, imatinib, sorafenib, lapatinib, etc.); or any combination thereof.

### V. Articles of Manufacture and Kits

The present disclosure also provides articles of manufacture, e.g., kits, comprising one or more containers (e.g., single-use or multi-use containers) containing a pharmaceutical composition of a ROR2 ARC described herein, optionally an additional therapeutic agent (which may be in the same or a separate pharmaceutical composition), and instructions for use. The ARC, and optional additional therapeutic agent, can be packaged separately in suitable packing such as a vial or ampule made from non-reactive glass or plastic. In certain embodiments, the vial or ampule holds a concentrated stock (e.g., 2×, 5×, 10× or more) of the ARC and optionally the additional therapeutic agent. In certain embodiments, the articles of manufacture such as kits include a medical device for administering the ARC and/or additional therapeutic agent (e.g., a syringe and a needle); and/or an appropriate diluent (e.g., sterile water and normal saline). The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Synthesis of Exemplary ARC Formulations

### A. ROR2 ARC Radiosynthesis Using p-SCN-Bn-DFO and [⁸⁹Zr]

The ARC [⁸⁹Zr]-DFO-Ab1 was formulated according to the scheme shown below.

### Part 1: Preconjugation of p-SCN-Bn-DFO

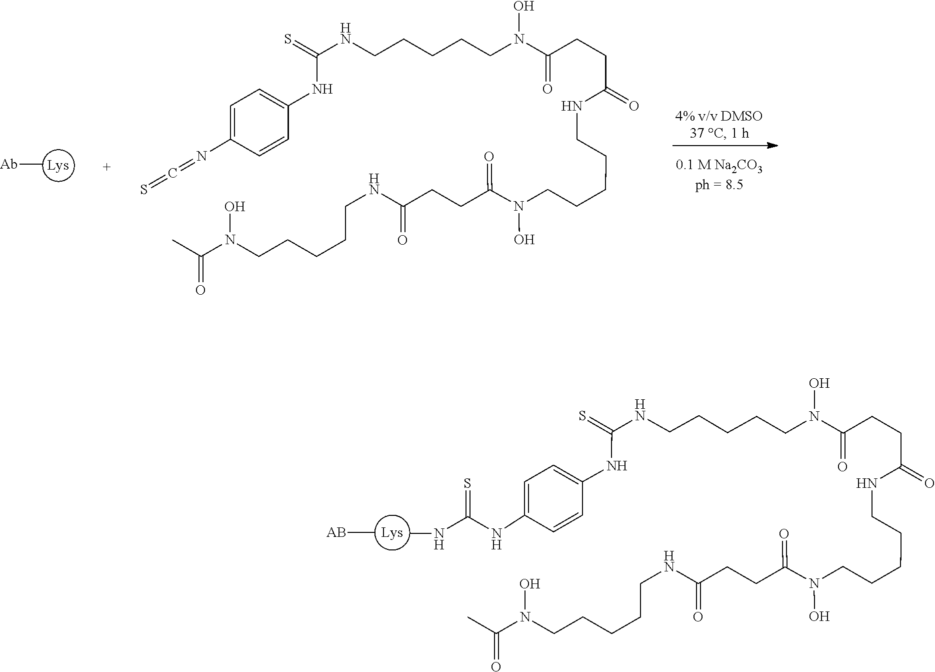

### Part 2: Radiochelation of Zirconium-89 Radionuclide

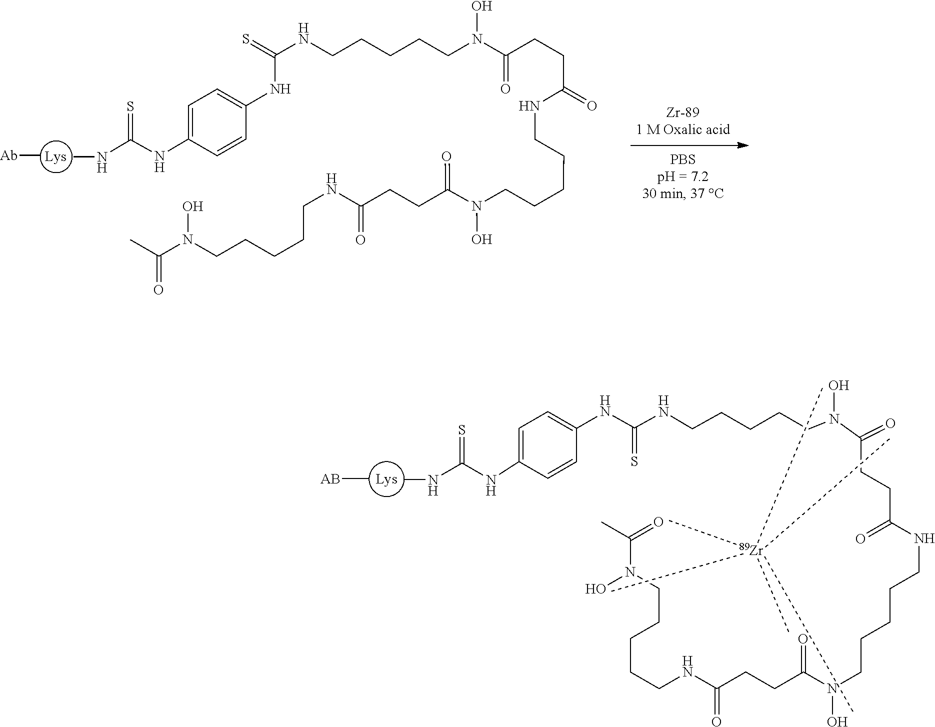

Pre-conjugation of the Ab with p-SCN-Bn-DFO was carried out by formulating the naked Ab in 10 mM acetate buffer [pH=5.5] into a reaction vial. The antibody solution was then buffered to a pH of 8.5 using 0.1 M sodium carbonate (Na₂CO₃). Next, DFO was solubilized in dimethyl sulfoxide (DMSO) at a concentration of 5-20 mg/mL. DFO was then added to the Ab reaction vial at the desired LAR stoichiometry so that the DMSO content does not exceed 5% v/v with the antibody volume. The DFO solution was mixed and reacted for 30 minutes at a temperature of 37° C. with agitation. During the reaction, a protein desalting 10 (PD-10) column was preconditioned in phosphate-buffered saline (PBS) (pH=7.2). Upon reaction completion, the reaction mixture was added to the PD-10 column and then Ab-DFO is eluted in the mobile phase (e.g., PBS, HEPES buffer or acetate buffer. For radiolabeling, Zr89 in 1 M oxalic acid was aliquoted into a reaction vial and then buffered with 1 M Na₂CO₃ to achieve a desired pH of 7.0 (required pH range is 6.8-7.5). The Ab-DFO was then added to the reaction vial and the labeling reaction is carried out at pH 7.0 and 37° C. for 30 minutes. At the end of the reaction, the [Zr89]-DFO-Ab was added to a PD-10 column preconditioned with a formulation buffer and then purified and eluted using the final formulation buffer. [Zr89]-DFO-Ab was then sterile-filtered to achieve its final form. A similar reaction process is performed for the alternative DFO linkers (e.g., DFO*, DFO-03, DFO-Km, and DF02).

### B. ROR2 ARC Radiosynthesis Using Boc₂-1,3,5-isoSGMTB and [I-124]

The ARC [I-124]-1,3,5-isoSGMIB-Ab1 may be formulated according to the scheme shown below.

### Part 1: Radiolabeling and Processing of boc-1,3,5-isoSGMTB Precursor

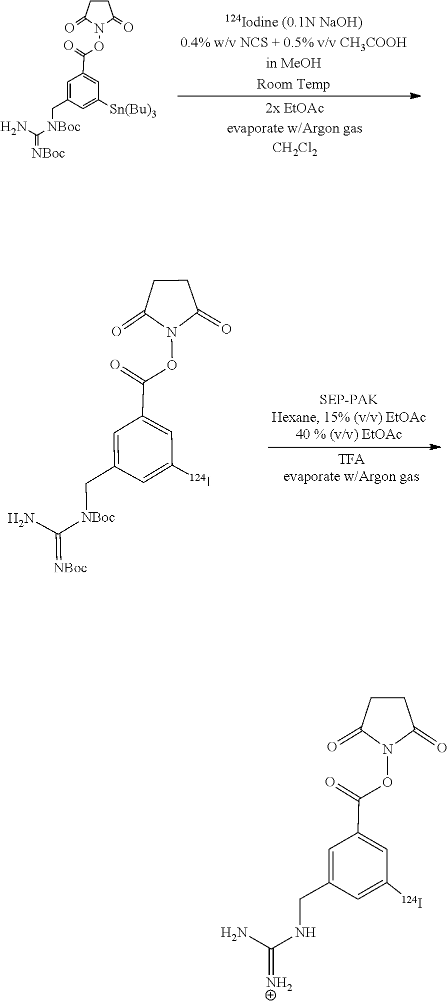

### Part 2: Bioconjugation of [I-124]-1,3,5-isoSGMIB to ROR2 mAb

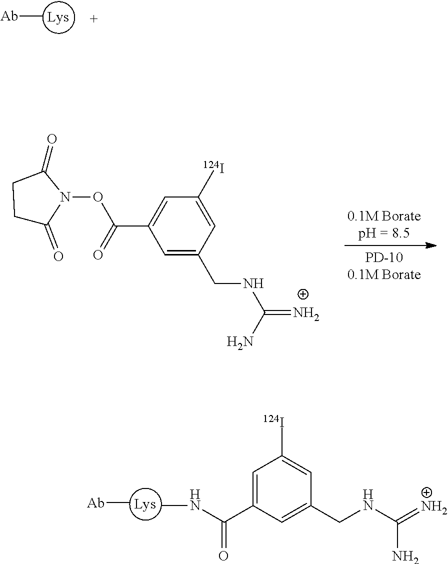

A solution of boc₂-isoSGMTB tri-butyl stannate precursor was prepared in a 100 μL solution of anhydrous methanol containing acetic acid (1-5%) and N-chlorosuccinimide (NCS) (2-8%). The precursor solution was then added to the dried ¹²⁴I activity, vortexed, and reacted at room temperature for 30 minutes. The methanol was then evaporated to dryness under a gentle nitrogen gas stream. Co-evaporation with ethyl acetate (100 μL) was performed twice to ensure complete dryness. The radiolabeled precursor was then reconstituted in dichloromethane and loaded onto an activated silica cartridge (650 mg, Waters). The cartridge was eluted with 30 mL of 100% hexane, 2×10 mL 15% ethyl acetate in hexane, and 10 mL of 40% ethyl acetate in hexane. The first 6 mL eluted by the 40% ethyl acetate in hexane were collected in fractions, pooled, and then dried under a gentle gas stream of nitrogen. Trifluoroacetic acid was then added to the vial and vortexed for 30 seconds. TFA was removed under a stream of nitrogen. Ab, prepared in borate buffer (pH=85), was then added to the reaction vial and allowed to react at 37° C. for 30 minutes. The labeled [¹²⁴I]-isoSGMIB-Ab was then purified over a PD-10 Sephadex resin column, eluted using the final formulation buffer, and sterile filtered.

### C. ROR2 ARC Radiosynthesis Using p-SCN-Bn-HOPO and [Zr89]

The ARC [Zr89]-HOPO-Ab1 was formulated according to the scheme shown below.

### Part 1: Preconjugation of p-SCN-Bn-HOPO

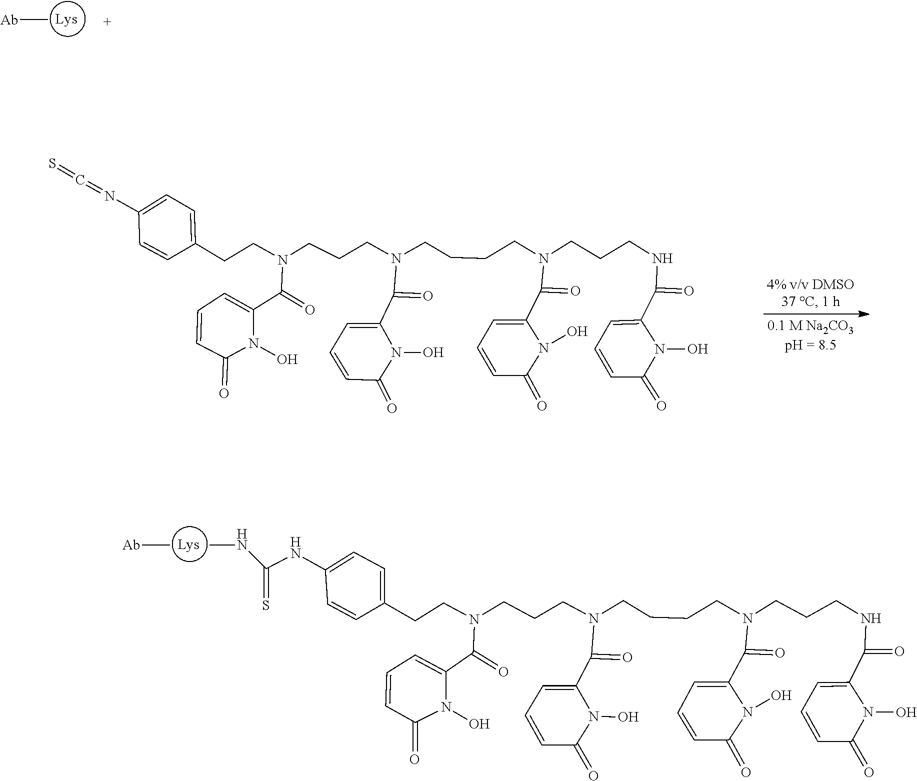

### Part 2: Radiochelation of Zirconium-89 Radionuclide

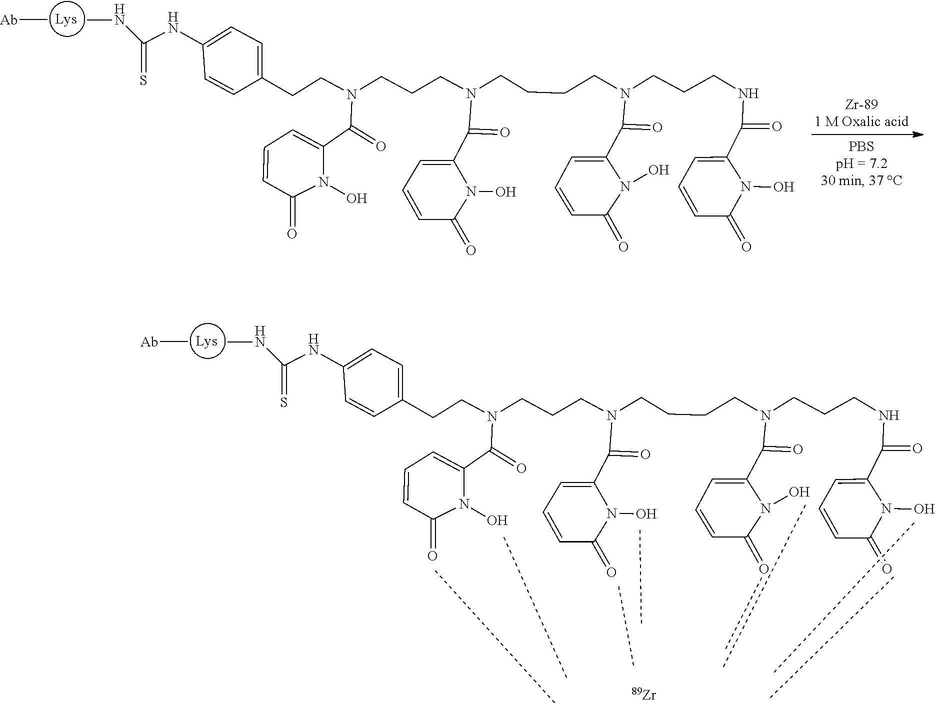

Pre-conjugation of the Ab with p-SCN-Bn-HOPO was carried out by formulating the naked Ab in 10 mM acetate buffer [pH=5.5] into a reaction vial. The antibody solution was then buffered to a pH of 9.0 using 0.1 M sodium carbonate (Na₂CO₃). Next, HOPO was solubilized in dimethyl sulfoxide (DMSO) at a concentration of 5-20 mg/mL. HOPO was then added slowly to the Ab reaction vial at the desired LAR stoichiometry so that the DMSO content did not exceed 5% v/v with the antibody volume. The HOPO solution was mixed and reacted for 60 minutes at a temperature of 37° C. with agitation. During the reaction, a protein desalting 10 (PD-10) column is preconditioned in phosphate-buffered saline (PBS) (pH=7.2). Upon reaction completion, the reaction mixture is added to the PD-10 column and then Ab-HOPO was eluted in the mobile phase (e.g., PBS, HEPES buffer or acetate buffer. For radiolabeling, Zr89 in 1 M oxalic acid is aliquoted into a reaction vial and then buffered with 1 M Na₂CO₃ to achieve a desired pH of 7.0 (required pH range is 6.8-7.5). The Ab-DFO was then added to the reaction vial and the labeling reaction is carried out at pH 7.0 and 37° C. for 2 hours. At the end of the reaction, 50 mM EDTA was added to scavenge any free Zr-89. At the end of the reaction, the [Zr89]-HOPO-Ab solution was added to a PD-10 column preconditioned with a formulation buffer and then purified and eluted using the final formulation buffer. [Zr89]-DFO-Ab was then sterile-filtered to achieve its final form.

### D. ROR2 ARC Radiosynthesis Using DOTA-NHS and [Zr89]

The ARC [Zr89]-DOTA-Ab1 may be formulated according to the scheme shown below.

### 2-Step Radiolabeling of DOTA Precursor for [Zr89]-DOTA-Ab

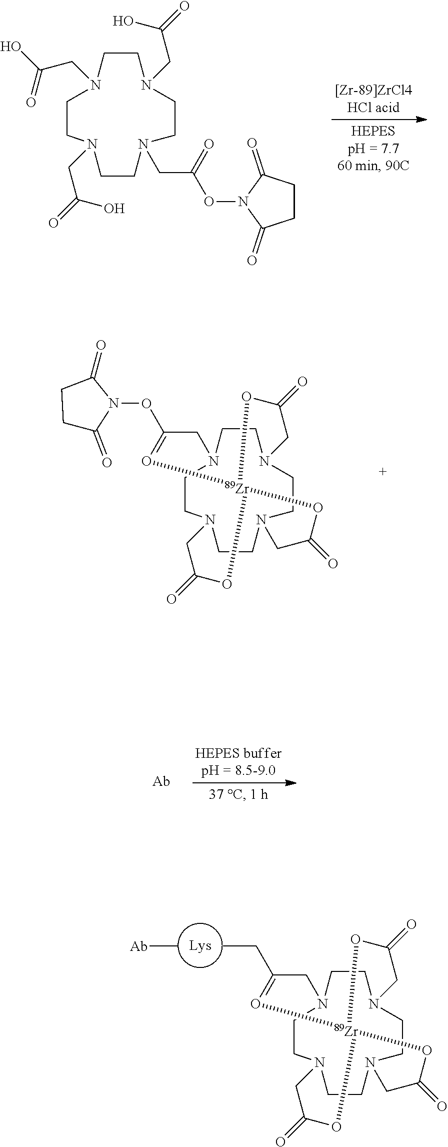

The 2-step method for labeling may be followed as described in Lyashchenko et al. Nuc Med Biol. (2024) 135:108943.

### E. ROR2 ARC Radiosynthesis Using boc₂-1,3,5-isoSGMTB and [At-211]

The ARC [At-211]-1,3,5-isoSAGMB-Ab1 may be formulated according to the scheme shown below.

### Part 1: Radiolabeling and Processing of boc₂-1,3,5-isoSGMTB Precursor

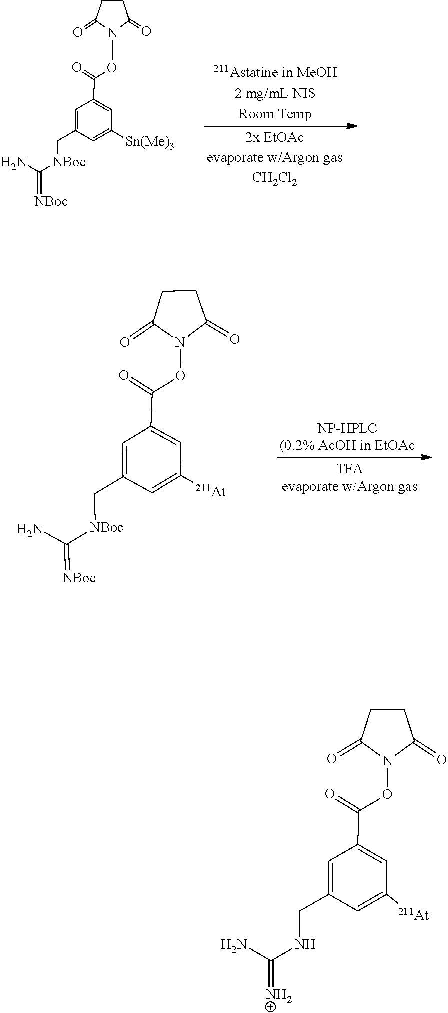

### Part 2: Bioconjugation of [At-211]-1,3,5-isoSAGMB to ROR2 mAb

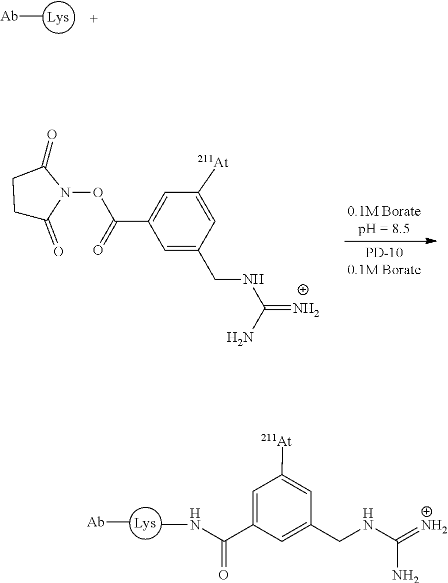

A solution of boc₂-isoSGMTB tri-butyl stannate precursor was prepared in a 100 μL solution of anhydrous methanol containing acetic acid (1-5%) and N-chlorosuccinimide (NIS) (2-8%). The precursor solution was then added to the ²¹¹At radioisotope in a methanol solution, vortexed, and reacted at room temperature for 30 minutes. The methanol was then evaporated to dryness under a gentle nitrogen gas stream. Co-evaporation with ethyl acetate (100 μL) was performed twice to ensure complete dryness. The radiolabeled precursor was then reconstituted in dichloromethane and loaded onto an activated silica cartridge (650 mg, Waters). The cartridge was eluted with 30 mL of 100% hexane, 2×10 mL 15% ethyl acetate in hexane, and 10 mL of 40% ethyl acetate in hexane. The first 6 mL eluted by the 40% ethyl acetate in hexane were collected in fractions, pooled, and then dried under a gentle gas stream of nitrogen. Trifluoroacetic acid was then added to the vial and vortexed for 30 seconds. TFA was removed under a stream of nitrogen. Ab, prepared in borate buffer (pH=85), was then added to the reaction vial and allowed to react at 37° C. for 30 minutes. The labeled [²¹¹At]-isoSAGMB-Ab was then purified over a PD-10 Sephadex resin column, eluted using the final formulation buffer, and sterile filtered.

### F. ROR2 ARC Radiosynthesis using boc₂-1,3,5-isoSGMTB and [I-131]

The ARC [I-131]-1,3,5-isoSGMIB-Ab1 may be formulated according to the scheme shown below.

### Part 1: Radiolabeling and Processing of boc₂-1,3,5-isoSGMTB Precursor

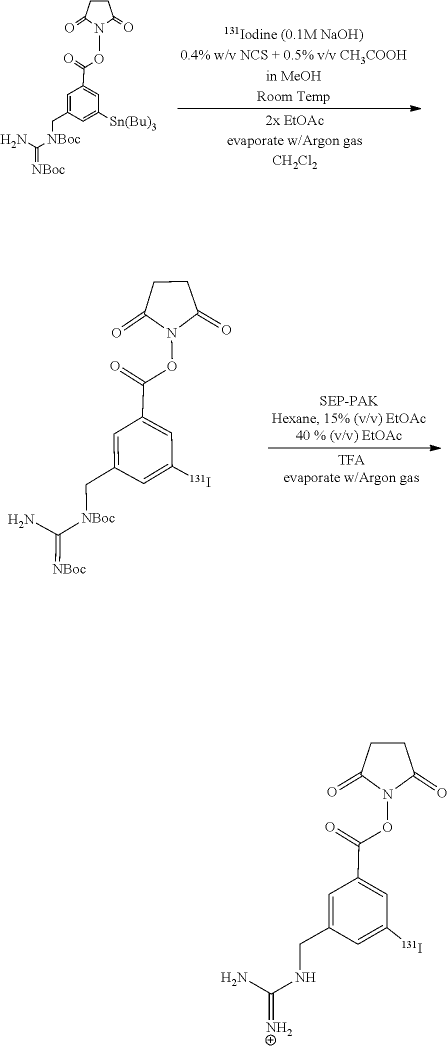

### Part 2: Bioconjugation of [I-131]-1,3,5-isoSGMIB to ROR2 mAb

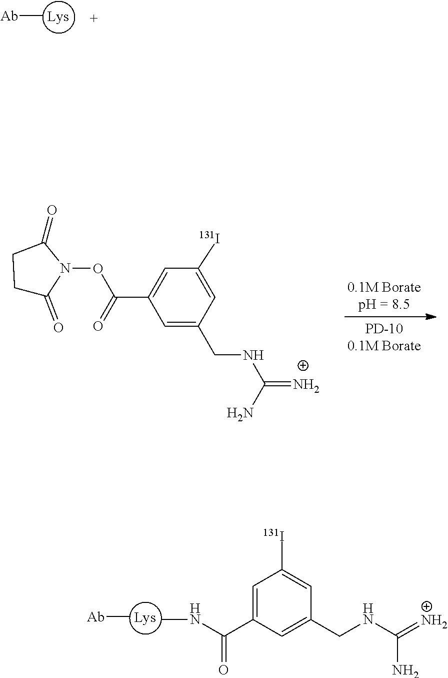

A solution of boc₂-isoSGMTB tri-butyl stannate precursor was prepared in a 100 μL solution of anhydrous methanol containing acetic acid (1-5%) and N-chlorosuccinimide (NCS) (2-8%). The precursor solution was then added to the dried ¹³¹I activity, vortexed, and reacted at room temperature for 30 minutes. The methanol was then evaporated to dryness under a gentle nitrogen gas stream. Co-evaporation with ethyl acetate (100 μL) was performed twice to ensure complete dryness. The radiolabeled precursor was then reconstituted in dichloromethane and loaded onto an activated silica cartridge (650 mg, Waters). The cartridge was eluted with 30 mL of 100% hexane, 2×10 mL 15% ethyl acetate in hexane, and 10 mL of 40% ethyl acetate in hexane. The first 6 mL eluted by the 40% ethyl acetate in hexane were collected in fractions, pooled, and then dried under a gentle gas stream of nitrogen. Trifluoroacetic acid was then added to the vial and vortexed for 30 seconds. TFA was removed under a stream of nitrogen. Ab, prepared in borate buffer (pH=85), was then added to the reaction vial and allowed to react at 37° C. for 30 minutes. The labeled [¹³¹I]isoSGMIB-Ab was then purified over a PD-10 Sephadex resin column, eluted using the final formulation buffer, and sterile filtered.

### G. ROR2 ARC Radiosynthesis Using boc₂-1,3,5-isoSGMTB and [I-125]

The ARC [I-125]-1,3,5-isoSGMIB-Ab1 may be formulated according to the scheme shown below.

### Part 1: Radiolabeling and Processing of boc₂-1,3,5-isoSGMTB Precursor

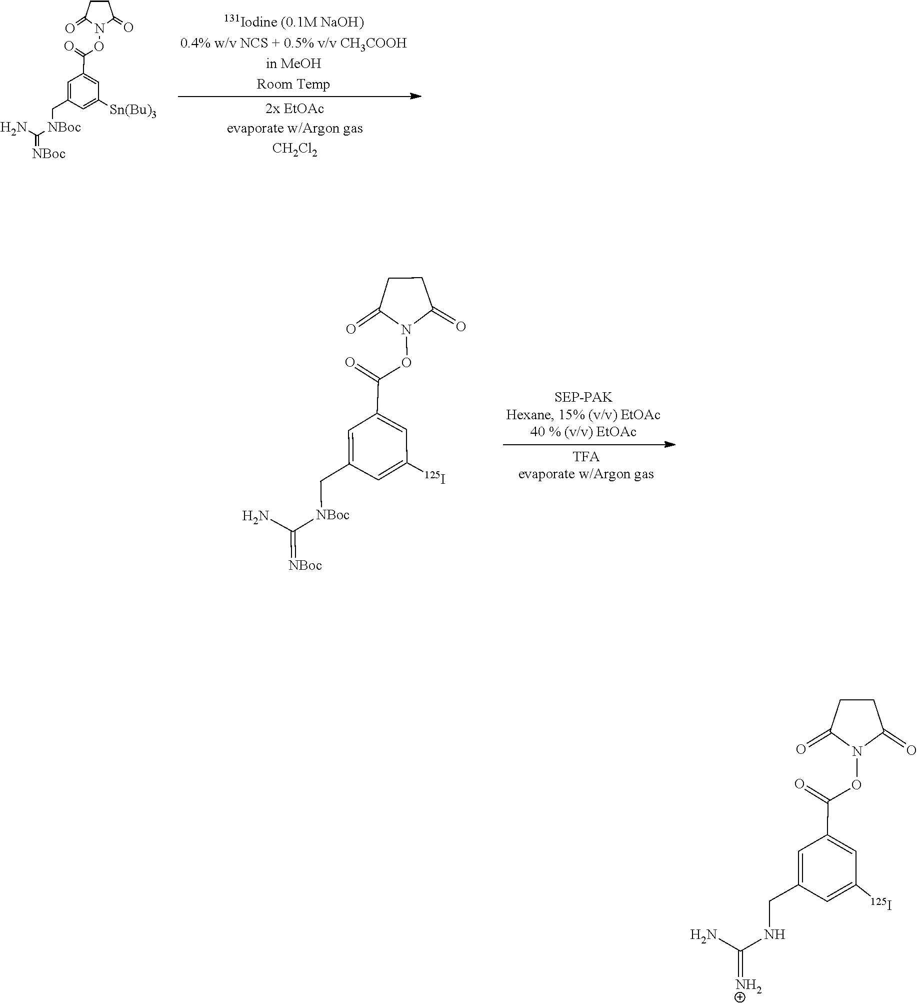

### Part 2: Bioconjugation of [I-125]-1,3,5-isoSGMIB to ROR2 mAb

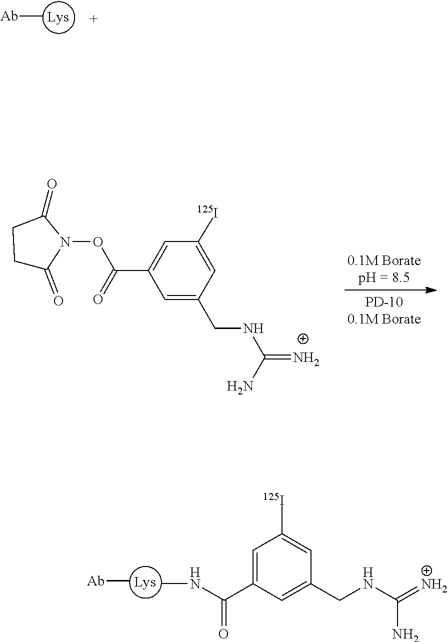

A solution of boc₂-isoSGMTB tri-butyl stannate precursor was prepared in a 100 μL solution of anhydrous methanol containing acetic acid (1-5%) and N-chlorosuccinimide (NCS) (2-8%). The precursor solution was then added to the dried ¹²⁵I activity, vortexed, and reacted at room temperature for 30 minutes. The methanol was then evaporated to dryness under a gentle nitrogen gas stream. Co-evaporation with ethyl acetate (100 μL) was performed twice to ensure complete dryness. The radiolabeled precursor was then reconstituted in dichloromethane and loaded onto an activated silica cartridge (650 mg, Waters). The cartridge was eluted with 30 mL of 100% hexane, 2×10 mL 15% ethyl acetate in hexane, and 10 mL of 40% ethyl acetate in hexane. The first 6 mL eluted by the 40% ethyl acetate in hexane were collected in fractions, pooled, and then dried under a gentle gas stream of nitrogen. Trifluoroacetic acid was then added to the vial and vortexed for 30 seconds. TFA was removed under a stream of nitrogen. Ab, prepared in borate buffer (pH=85), was then added to the reaction vial and allowed to react at 37° C. for 30 minutes. The labeled [¹²⁵I]isoSGMIB-Ab was then purified over a PD-10 Sephadex resin column, eluted using the final formulation buffer, and sterile filtered.

### Example 2: Production of [Zr89]-DFO-Ab1

The lead antibody Ab1 was initially formulated in PBS, pH 7.2 and buffered with 0.1 M Na₂CO₃ to a pH range of 8.5-9.0. To this solution was added p-SCN-Bn-Deferoxamine (DFO) solubilized in DMSO (4% v/v). The pre-conjugated antibody was incubated with DFO for one hour at 37° C. and purified via a PD-10 (Cytiva) desalting column (PBS, pH 7.2). The purity and quality of the pre-conjugated antibody was assessed via radio high-pressure liquid chromatography-size exclusion chromatography (radioHPLC-SEC).

Two reaction conditions were tested to effectively label the pre-conjugated antibody with radioactive Zirconium-89 ([Zr89]). Pre-conjugated Ab1 (buffered in PBS buffer) was incubated at room temperature for one hour with a solution of radioactive [Zr89] in 1M oxalic acid that was either unbuffered (pH~2.0) or buffered with Na₂CO₃ to achieve pH~7.0. The resulting [Zr89]-DFO-Ab1 was purified and analyzed via radio thin layer chromatography (radioTLC). The [Zr89] buffered to pH~7.0 showed roughly 64% labeling of the antibody by radioTLC, compared to 5% when unbuffered.

To test the effect of incubation solution buffering on radiolabeling efficiency, DFO-Ab1 (buffered in PBS buffer) was incubated with the [Zr89] solution (buffered with Na₂CO₃ to pH 7) in a solution buffered with either PBS buffer or 10 mM acetate buffer. After one hour of incubation at room temperature, the acetate buffer condition achieved 65% labeling efficiency by radioTLC, compared to 35% in PBS buffer by radioTLC.

To determine the effect of DFO conjugation on radiolabeling efficiency, Ab1 was pre-conjugated to DFO at six stoichiometric Drug to Antibody Ratios (DARs). The radiochemical yield (RCY) of the reaction crude from each DFO-Ab1 ratio was quantified by radioTLC analysis. The radiolabeling efficiency of the purified [Zr89]-DFO-Ab1 mixtures appeared to increase with decreasing ratios. The stoichiometric ratio of 1:8 (DAR~4) was deemed optimal with radiolabeling efficiency of 93.1% by radioTLC.

To test the effect of the antibody buffer on radiolabeling efficiency, pre-conjugated DFO-Ab1 was buffered in 10 mM sodium acetate+sucrose 5% m/v at either pH 7.0 or 5.5. These solutions were incubated with [Zr89](buffered to pH 7 in Na₂CO₃) at room temperature for one hour. Both solutions achieved 95.4% radiolabeling efficiency by radioTLC.

To reduce radioactive dose exposure to chemists and limit the amount of radioactive decay experienced by the produced ARC, alternate conditions for temperature and radiolabeling time were tested. DFO-Ab1 with a DAR of 4 was radiolabeled at either room temperature or 37° C. for either 10 minutes or 20 minutes. The radiolabeling efficiency of each of these conditions exceeded 95% when tested by radioTLC.

### Example 3: Formulation Stability Assessment of [Zr89]-DFO-Ab1

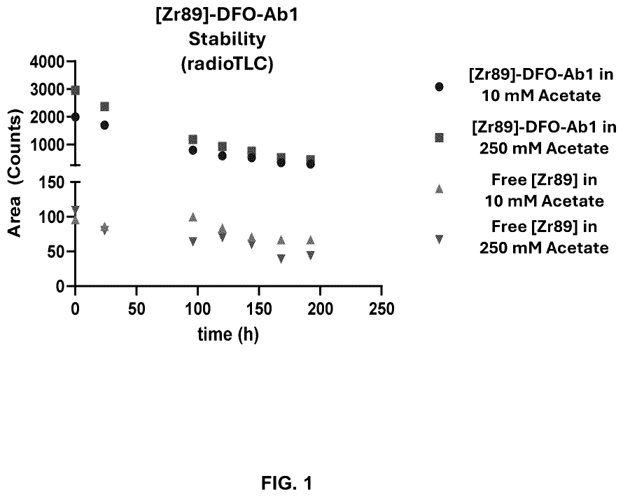

To determine the stability of the radioactive [Zr89]isotope in the DFO-Ab1 ARC formulation, two buffer formulations were evaluated based on clinical precedence: 10 mM or 250 mM acetate buffers, both supplemented with gentisic acid as a radiolytic stabilizing excipient. The purity of the resulting ARC was tested by radioTLC 0, 24, 96, 120, 144, 168, and 192 hours after radiolabeling (FIG. 1). In both conditions, the ARC radioactivity signal decay was consistent with physical isotope decay. No release of [Zr89] from the radiolabeled ARC was observed for either formulation and the observed free [Zr89] signal was consistent with background noise signal on the TLC plate.

### Example 4: Assessment of [Zr89]-DFO-Ab1 Cell Binding and Internalization Ability at Different Linker-to-Antibody Ratios

To test the effect of the DFO/Ab1 Linker to Antibody Ratio (LAR) on cell binding, Ab1 was conjugated with DFO at varying linker-to-antibody ratios. The binding affinity of each conjugate was then tested in a cellular binding assay utilizing the ROR2-expressing, human colon cancer cell line HCT-116. For each conjugate with variable LAR, concentration dilution series was prepared, ranging from 1 mM down to 0.01 nM. A naked Ab control was also prepared as a LAR=0 control. Samples were incubated with the HCT-116 cells on ice for 30 minutes, washed, and then were incubated with phycoerythrin anti-IgG detection antibody. Cells were additionally washed and then assayed for mean fluorescent intensity (MFI) using a flow cytometer. The resulting MFI for each sample (run in triplicate) was then plotted against concentration. Using a sigmoidal non-linear regression in GraphPad Prism, binding affinity was assess as a half-maximal effective concentration (EC₅₀).

Free Ab1 was compared to LARs of 1, 2, and 4; little difference in binding affinity was observed at any of the LAR ratios, each approximately 4-6 nM (Table 2).

TABLE 2
Cell Binding Affinity of ARC at Various LARs

| Antibody Drug Conjugate | EC₅₀ (nM) |
|---|---|
| Ab1 | 4.247 |
| DFO-Ab1 LAR ~1 (2eq) | 5.018 |
| DFO-Ab1 LAR ~2 (4eq) | 6.400 |
| DFO-Ab1 LAR ~4 (8eq) | 6.370 |
| Ab1 | 2.151 |
| DFO-Ab1 LAR ~1 (2eq) | 2.041 |
| DFO-Ab1 LAR ~2 (4eq) | 2.436 |
| DFO-Ab1 LAR ~4 (8eq) | 1.884 |
| Ab1 | 1.525 |
| DFO-Ab1 LAR ~1 (2eq) | 1.826 |
| DFO-Ab1 LAR ~5 (10eq) | 3.265 |
| DFO-Ab1 LAR ~10 (20eq) | 4.079 |

Further, the average cell internalization rate in HCT-116 cells was measured for the DFO-Ab1 conjugates with various LARs. The internalization rate was measured about 0, 0.5, 2. 3. And 4 hours of incubation at 37° C. (Table 3).

TABLE 3
Cell Internalization of ARC at Various LARs in HCT-116 Cells

| | | | DFO-Ab1 | | DFO-Ab1 | | DFO-Ab1 | |
|---|---|---|---|---|---|---|---|---|
| | Free Ab1 | | LAR ~1 | | LAR ~2 | | LAR ~4 | |
| Incubation | Avg | StDev | Avg | StDev | Avg | StDev | Avg | StDev |
| Time | (%) | (%) | (%) | (%) | (%) | (%) | (%) | (%) |
| 4 hr, 37° C. | 30.4 | 3.2 | 23.0 | 1.4 | 27.6 | 0.7 | 21.0 | 4.1 |
| 3 hr, 37° C. | 32.1 | 4.2 | 24.8 | 5.3 | 33.2 | 2.7 | 21.4 | 3.8 |
| 2 hr, 37° C. | 30.3 | 0.3 | 23.9 | 4.9 | 27.6 | 0.7 | 20.8 | 1.8 |
| 0.5 hr, 37° C. | 25.9 | 1.8 | 19.6 | 4.7 | 22.3 | 0.5 | 14.5 | 7.2 |
| 0 hr, 37° C. | 0.0 | 0.0 | 0.0 | 0.0 | 0.0 | 0.0 | 0.0 | 0.0 |
| | | | DFO-Ab1 | | DFO-Ab1 | | DFO-Ab1 | |
| | Free Ab1 | | LAR ~1 | | LAR ~5 | | LAR ~10 | |
| Incubation | Avg | StDev | Avg | StDev | Avg | StDev | Avg | StDev |
| Time | (%) | (%) | (%) | (%) | (%) | (%) | (%) | (%) |
| 4 hr, 37° C. | 28.1 | 12.8 | 35.5 | 3.8 | 31.6 | 6.0 | 34.3 | 2.1 |
| 3 hr, 37° C. | 27.2 | 1.9 | 29.8 | 3.3 | 24.5 | 4.9 | 30.6 | 3.1 |
| 2 hr, 37° C. | 27.9 | 4.8 | 28.4 | 4.4 | 20.4 | 6.3 | 25.7 | 2.7 |
| 0.5 hr, 37° C. | 22.7 | 16.9 | 9.2 | 5.9 | 10.8 | 4.1 | 14.4 | 4.5 |
| 0 hr, 37° C. | 0.0 | 0.0 | 0.0 | 0.0 | 0.0 | 0.0 | 0.0 | 0.0 |

As a negative control, the cell internalization rate was also measured in AsPc-1 pancreatic cancer cells (ROR2 negative) for free Ab1 compared to DFO-Ab 1 with LAR 4 (Table 4). No internalization of free Ab1 or DFO-Ab1 was observed in AsPc-1 cells, as expected.

TABLE 4
Cell Internalization of Free Ab1 versus DFO-Ab1 in AsPc-1 Cells

| | Replicate 1 | | | Replicate 2 | | | Replicate 3 | | |
|---|---|---|---|---|---|---|---|---|---|
| Incubation | Raw | | | Raw | | | Raw | | |
| Time | MFI | SD | Count | MFI | SD | Count | MFI | SD | Count |

Free Ab1

| | | | | | | | | | |
|---|---|---|---|---|---|---|---|---|---|
| 4 hr, 37° C. | 61 | 77.99 | 10,000 | 68 | 359.57 | 10,000 | 78 | 1324.21 | 10,000 |
| 3 hr, 37° C. | 67 | 606.61 | 10,000 | 81 | 1330.98 | 10,000 | 70 | 349.05 | 10,000 |
| 2 hr, 37° C. | 64 | 83.33 | 10,000 | 64 | 94.53 | 10,000 | 73 | 807.29 | 10,000 |
| 0.5 hr, 37° C. | 68 | 588.96 | 10,000 | 70 | 822.49 | 10,000 | 84 | 1462.66 | 10,000 |
| 0 hr, 37° C. | 75 | 1514.78 | 10,000 | 59 | 32.95 | 10,000 | 77 | 1084.41 | 10,000 |
| 2^{nd} det | 54 | 17.12 | 10,000 | 53 | 18.86 | 10,000 | 54 | 20.3 | 10,000 |
| Ab only | | | | | | | | | |
| Unstained | 54 | 18.29 | 10,000 | 52 | 19.86 | 9682 | 54 | 18.12 | 10,000 |

DFO-Ab1 DAR 4 (8eq)

| | | | | | | | | | |
|---|---|---|---|---|---|---|---|---|---|
| 4 hr, 37° C. | 115 | 3045.76 | 10,000 | 76 | 1230.89 | 10,000 | 66 | 452.99 | 10,000 |
| 3 hr, 37° C. | 64 | 225.79 | 10,000 | 68 | 753.24 | 10,000 | 91 | 1545.62 | 10,000 |
| 2 hr, 37° C. | 83 | 2021.84 | 10,000 | 88 | 1655.63 | 10,000 | 62 | 65.85 | 10,000 |
| 0.5 hr, 37° C. | 110 | 2659.28 | 10,000 | 63 | 36.19 | 10,000 | 62 | 66.58 | 10,000 |
| 0 hr, 37° C. | 63 | 60.26 | 10,000 | 63 | 58 | 10,000 | 62 | 53.87 | 10,000 |
| 2^{nd} det | 54 | 17.46 | 10,000 | 54 | 17.65 | 10,000 | 53 | 17.38 | 10,000 |
| Ab only | | | | | | | | | |
| Unstained | 54 | 17.87 | 10,000 | 54 | 17.91 | 10,000 | 53 | 17.22 | 10,000 |

### Example 5: Bioactivity of [Zr89]-DFO-Ab1

### Materials and Methods

Radio high-pressure liquid chromatography-size exclusion chromatography (radioHPLC-SEC) was used to assess the integrity of Ab1 post-labeling via its immunoreactivity with the ROR2 antigen. In this method, [Zr89]-DFO-Ab1 was mixed with an excess of ROR2-His6 antigen. The mixture was allowed to react for 15 minutes at ambient conditions. At that point, a sample of ~40 μCi was loaded onto a HPLC system and analyzed via size exclusion on a Advance Bio 300A column in a PBS mobile phase. If Ab1 retains its immunoreactivity, it will fully complex with the ROR2 antigen resulting in a 2-3 minute shift in the gamma trace of the radioHPLC. If it fully complexes, no activity signal will be observed at characteristic ARC peak (~9.5-10 min). If Ab1 is damaged by the process, or if the mass ratio of ROR2 antigen is insufficient, then characteristic ARC peak will remain. If Ab1 remains undamaged then it will bind into an antibody-antigen complex when incubated with excess ROR2 antigen that is visible as a peak shift via radioHPLC-SEC. The antigen does not appear in the readout, only the radiolabeled ARC and ARC-ROR2 complex are detectable.

### Results

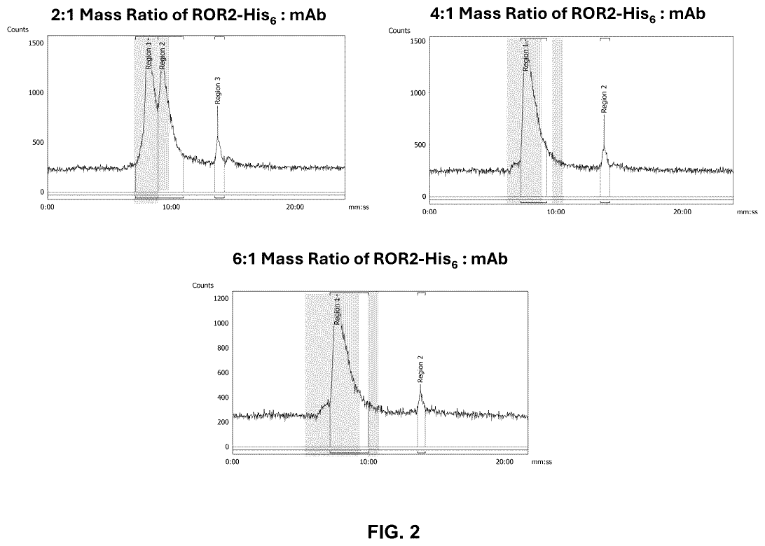

Using radioHPLC-SEC, the ratios of ROR2 antigen to ARC were evaluated to see what conditions are need for establishing an immunoreactivity assay for assessing the binding affinity of [Zr89]-DFO-Ab1 post radiolabeling. A mass ratio of 2:1 showed incomplete antigen complexation, but ratios of 4:1 and 6:1 showed complete complexation of the ARC to the antigen, validating the ARCs bioactivity and setting a minimum threshold for further immunoreactivity assays. (FIG. 2).

### Example 6: Preclinical Imaging with [Zr89]-DFO-Ab1

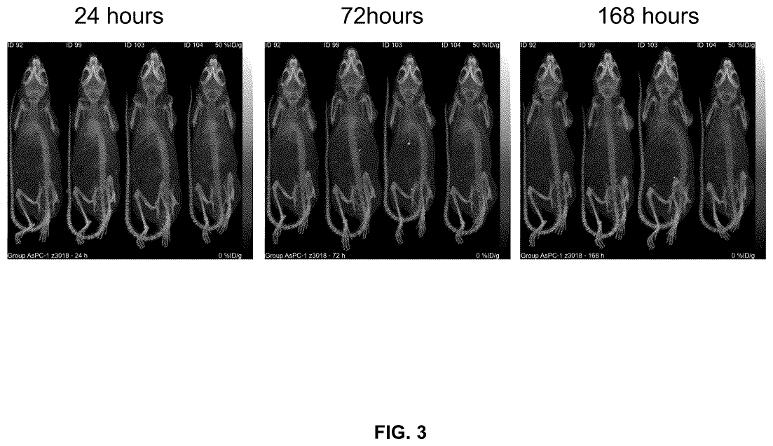

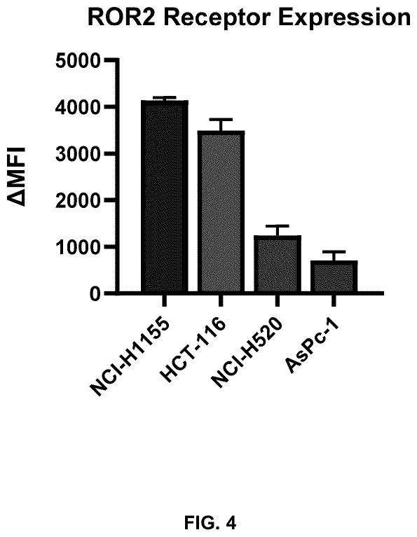

To ascertain the relative biodistribution and non-specific tumor uptake of [Zr89]-DFO-Ab1, the ARC formulation was administered to mice bearing ROR2-negative tumors (AsPc-1). The mice were imaged using micro positron emission tomography/computed tomography (microPET/CT) at timepoints of 24, 72, and 168 hours after administration. At all timepoints, the blood pool was the predominant factor for uptake in the mice. Little to no uptake was observed in tumors (FIG. 3).

To calculate the dosimetry in humans for handling the ARC formulation, ROI quantification results were converted into time-integrated activity concentrations (TIAC) for human male and female tissues (Tables 5 and 6). The uptake was calculated as % ID human=% ID animal and the concentration was calculated as % ID human=% ID/g animal×animal BW×HumanPhantomOrganWeight/HumanPhantomBodyWeight). The Remainder Body was calculated as Remainder Body=Total Body−(sum of all source organs) where tumor activity is lumped into the remainder body.

The following assumptions were made in the calculations: activity in all source organs were considered to be 0 at 0 hours post injection (with 100% in the remainder body); uptake/concentration was assumed to increase linearly from 0 to 24 hours using the 24 hour measured value; red marrow was calculated by mapping from left ventricle (blood) assuming concentration ratio between red marrow and blood to be 0.36; AUC from 0 to final time point is calculated using trapezoidal integration; all fitting is done with physical decay removed (fitting only the biological clearance) and then physical decay is reincorporated.

TABLE 5
Human Dosimetry Calculations (Male)

| Adult Human | Mean Absorbed |
|---|---|
| Male Tissue | Dose (mGy/MBq) |
| Adrenals | 0.335 ± 0.010 |
| Brain | 0.152 ± 0.003 |
| Esophagus | 0.303 ± 0.009 |
| Eyes | 0.189 ± 0.006 |
| Gallbladder Wall | 0.352 ± 0.010 |
| Heart Wall | 0.655 ± 0.019 |
| Kidneys | 0.352 ± 0.013 |
| Left colon | 0.315 ± 0.010 |
| Liver | 0.412 ± 0.014 |
| Lungs | 0.326 ± 0.011 |
| Osteogenic Cells | 0.370 ± 0.013 |
| Pancreas | 0.332 ± 0.010 |
| Prostate | 0.278 ± 0.010 |
| Rectum | 0.283 ± 0.010 |
| Red Marrow | 0.301 ± 0.009 |
| Right colon | 0.317 ± 0.010 |
| Salivary Glands | 0.234 ± 0.008 |
| Small Intestine | 0..315 ± 0.010 |
| Spleen | 0.330 ± 0.010 |
| Stomach Wall | 0.317 ± 0.010 |
| Testes | 0.225 ± 0.008 |
| Thymus | 0.320 ± 0.009 |
| Thyroid | 0.256 ± 0.009 |
| Total Body | 0.242 ± 0.008 |
| Urinary Bladder Wall | 0.258 ± 0.009 |

TABLE 6
Human Dosimetry Calculations (Female)

| Adult Human | Mean Absorbed |
|---|---|
| Female Tissue | Dose (mGy/MBq) |
| Adrenals | 0.411 ± 0.012 |
| Brain | 0.193 ± 0.005 |
| Breasts | 0.250 ± 0.008 |
| Esophagus | 0.351 ± 0.010 |
| Eyes | 0.235 ± 0.007 |
| Gallbladder Wall | 0.367 ± 0.011 |
| Heart Wall | 0.779 ± 0.023 |
| Kidneys | 0.428 ± 0.015 |
| Left colon | 0.407 ± 0.012 |
| Liver | 0.491 ± 0.016 |
| Lungs | 0.404 ± 0.013 |
| Osteogenic Cells | 0.418 ± 0.014 |
| Ovaries | 0.353 ± 0.012 |
| Pancreas | 0.400 ± 0.012 |
| Rectum | 0.344 ± 0.012 |
| Red Marrow | 0.369 ± 0.011 |
| Right colon | 0.377 ± 0.012 |
| Salivary Glands | 0.263 ± 0.009 |
| Small Intestine | 0.346 ± 0.011 |
| Spleen | 0.407 ± 0.012 |
| Stomach Wall | 0.367 ± 0.011 |
| Thymus | 0.382 ± 0.011 |
| Thyroid | 0.288 ± 0.009 |
| Total Body | 0.300 ± 0.010 |
| Urinary Bladder Wall | 0.252 ± 0.009 |
| Uterus | 0.345 ± 0.012 |

### Example 7: Assessment of [Zr89]-DFO-Ab1 Cellular Binding and Rate of Internalization

### Materials andMethods

### Cellular Binding Studies

Free Ab1 antibody was conjugated with the p-SCN-Bn-DFO (DFO) chelating linker at the optimized ratio (LAR~4) to form the antibody-linker complex, DFO-Ab1. This complex was then assessed for receptor binding efficiency and rate of internalization in ROR2-positive NCI-H1155 and HCT-116 cell lines, as well as ROR2-negative AsPc-1 cell line.

Test articles were conjugated with the were prepared in advance at a 2× stock concentration for the desired test article concentration range of 1000 nM-0.1 nM using a 10-fold serial dilution. An intermediate 2 μM stock of each test article was created in its native solution. The 2000 nM test solution was then made by adding 100 μL of the 2 μM intermediate stock with 100 μL of staining solution. The remaining test solutions, 200 nM-0.02 nM, were then created by performing a serial dilution of 20 μL from the 2000 nM test article with 180 μL of staining buffer until all test article concentrations were made. The test articles were then kept cold at 4° C.

Cells were harvested using trypsin-EDTA (0.25%), washed with cold PBS, and resuspended at a concentration of 5×106 cells/mL in cold staining buffer. 50 μl aliquots containing 2.5×105 cells were added to 24 wells of a 96-well, U-bottom plate (ThermoFisher Cat. No. 268200) and kept on ice (~4° C.). 50 μL of each respective test article concentration series was then gently mixed in wells containing cells. Triplicates of each condition were plated. In addition, three control conditions were plated in triplicate for cells. An unstained control cells were plated that received no test article and no detection antibody staining. For a non-specific staining control, cells received no test article but did receive secondary detection antibody staining. Lastly, isotype control cells received 50 μL of the IgG1 isotype control antibody and the detection antibody.

The treated cells were then incubated at 4° C. for 20 minutes. Cells were then thrice washed by centrifugating the plate at 500 g for 3 minutes, aspirating the supernatant, and then gently washing the cells in 200 μL of wash buffer. After the final centrifugation step, cells were resuspended in 100 μL of secondary detection antibody solution, excluding the unstained control cell samples, and incubated for another 20 minutes at 4° C. Cell washing was thrice repeated under the same washing conditions. After the third wash, cells were resuspended in 100 μL of fixation buffer and incubated at room temperature for 10 minutes. Cells were then washed one final time and resuspended in 200 μL of FACS buffer. Cells were then stored at 4° C. until ready for flow cytometry analysis.

FACS analysis was performed to assess mean fluorescent intensity (MFI) using the Attune Nex Flow Cytometer (ThermoFisher) using the YL1 laser channel (280 nm). Voltage settings for forward scatter and side scattering were determined independently for each individual cell line using their respective unstained control cells. Voltage settings for the YL1 laser channel was determined using the isotype control cells. The raw MFI values for each test condition were assessed by measuring fluorescent signal for 10,000 single cell events after gating for intact, single cells. The MFI of each test article sample was calculated by measuring the raw MFI value of the sample and subtracting the background MFI value as determined by the non-specific staining control cells. MFI values were then plotted vs. test article concentration in GraphPad Prism and fitted with a sigmoidal 4PL curve to calculate the cell binding EC50 value.

### Cellular Internalization Studies

For cellular internalization studies, cells were harvested using trypsin-EDTA (0.25%), washed with cold PBS, and resuspended at a concentration of 5×106 cells/mL in cold staining buffer. Ab1 and DFO-Ab1 conjugates were then diluted to create 2× stock solutions of each test article at a concentration of 126.8 μM. For each cell line, 1.2 mL of cell solution was transferred to a 15 mL conical tube and then independently mixed with 1.2 mL of each respective test article. The tubes were then incubated on ice for 20 minutes with gentle agitation to allow for saturated cell surface binding. After 20 minutes, the cells were thrice washed by centrifuging the tubes at 500 g for 3 min, aspirating the supernatant, and then washing in ice-cold wash buffer. After final washing, cells in each tube were resuspended in staining buffer.

50 μl aliquots containing 2.5×105 cells were added to 24 wells of a 96-well, U-bottom plate (ThermoFisher Cat. No. 268200) to allow for testing of 8 conditions in triplicate, and incubated at 37° C. for 0, 30, 120, 180, or 240 minutes. Conditions groups comprised cells incubated with one of the following test articles:
Unstained control with assay buffer
Secondary detection antibody only
Isotype control antibody only
Ab1 or a DFO-Ab1 conjugate at 833 nM.

Cell samples were incubated for their predetermined length of time, then collected to be centrifuged at 250×g and washed thrice with FACS buffer and resuspended in 100 μL of FACS buffer.

A 10× stock of secondary detection antibody was diluted 1:500 in FACS buffer. 10 μL of the secondary detection antibody was then added to the appropriate tubes. Cells were incubated on ice for 20 minutes, washed thrice with FACS buffer and resuspended in 250 μL of fixation buffer. The samples were stored at 4° C. until ready for FACS analysis.

FACS analysis was performed to assess mean fluorescent intensity (MFI) using the Attune Nex Flow Cytometer (ThermoFisher) using the YL1 laser channel (280 nm). Voltage settings were determined independently for each individual cell line. MFI values for each test condition were assessed by measuring fluorescent signal for 10,000 single cell events. The relative internalization of Ab1 or Ab1-DFO at each timepoint was then determined by subtracting the mean background MFI, measured in the control arms, and comparing it with its applicable control at time 0.

All studies were biologically replicated 2-3 times to ensure reproducibility and reliability of observed results.

### Results

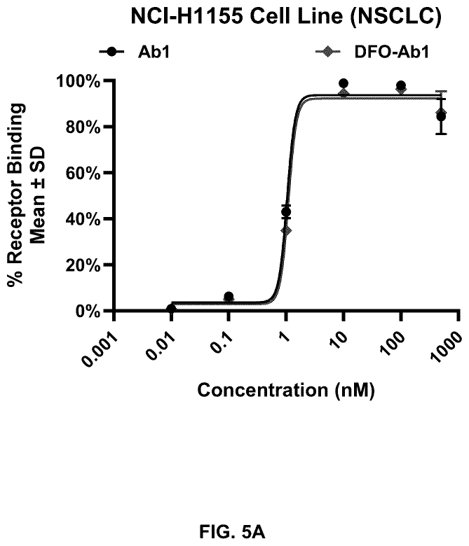

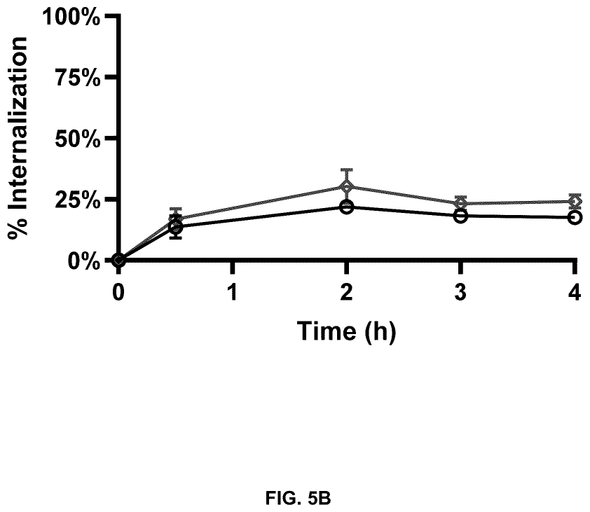

Free Ab1 was compared to DFO-Ab1 in NCI-H1155 cell line. No substantial difference in binding was observed at any concentrations, with both exhibiting the same binding EC₅₀ (FIG. 5A). In addition, no substantial difference in the rate of internalization was observed between the two compositions during a period of 4 hours (FIG. 5B).

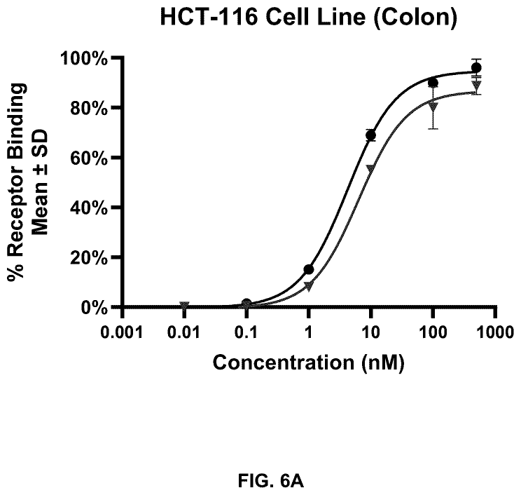

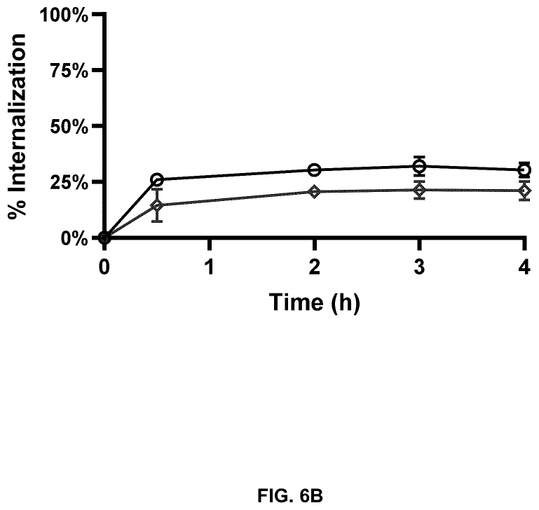

Free Ab1 was compared to DFO-Ab1 in HCT-116 cell line. No substantial difference in binding was observed at any concentrations, with both exhibiting the same binding EC₅₀ (FIG. 6A). In addition, no substantial difference in the rate of internalization was observed between the two compositions during a period of 4 hours (FIG. 6B).

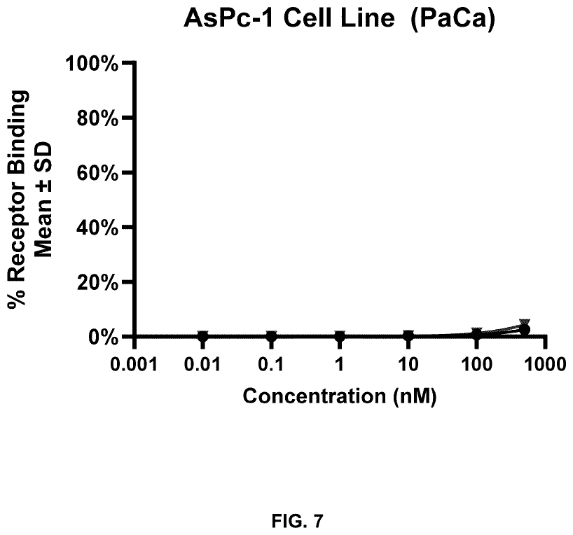

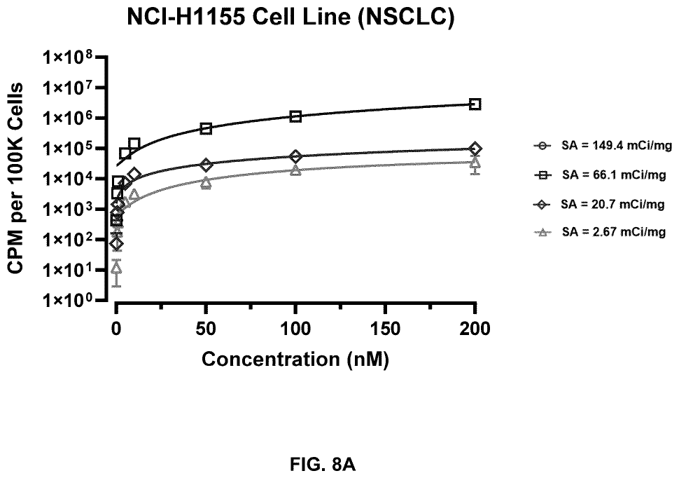

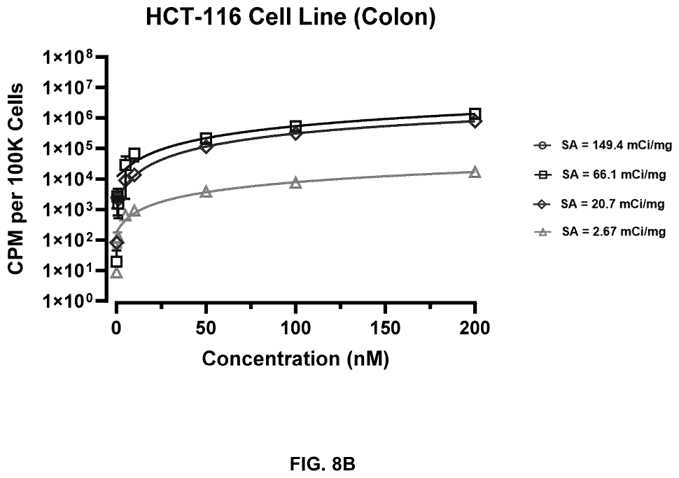

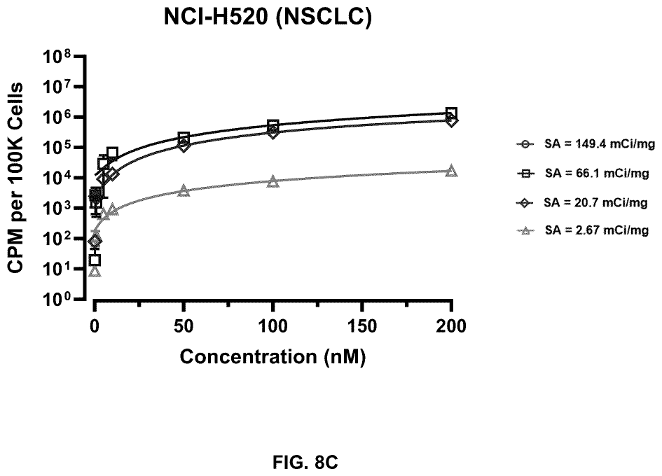

As a negative control, the cellular binding and internalization rate was also measured in AsPc-1 pancreatic cancer cells (ROR2 negative) for free Ab1 compared to DFO-Ab1 with DAR 4. In cell binding studies, neither test article showed any binding at concentrations up to 500 nM (FIG. 7). No internalization of free Ab1 or DFO-Ab1 was observed in AsPc-1 cells, as expected (Table 4).

### Example 8: Assessment of [Zr89]-DFO-Ab1 Binding Affinity Upon Zr-89 Radiolabeling

### Materials and Methods

In this series of experiments, the binding affinity of [Zr89]-DFO-Ab1 was assessed by incubating ROR2 expressing cells with serial dilutions of the ARC and gamma counting the bound radioactivity. The experiments were performed in cell lines representing various solid tumor types and a range of ROR2 expression.

Zr-89 radionuclide in 1M oxalic acid was added to a reaction vial, typically 10-50 μL depending on batch specific activity. 150 μL of PBS for every 10 μL was then added to the reaction vial. The pH of the Zr-89 solution was then adjusted using Na₂CO₃ (ThermoFisher Cat. No. 436800250) to a desired pH of 7.0±0.5. DFO-Ab1 was then added to the reaction at a range of 0.4-3 mg, depending on the desired specific activity. The solution was allowed to react for 30 minutes at 37° C. and 350 rpm on a thermomixer.

After completion of the radiolabeling reaction, the mixture was transferred to a PD-10 column (Cytivia Cat. No. 17085101) that was prepared using 250 mM sodium acetate buffer (Sigma Aldrich Cat No S2889). 1.5 mL of 250 mM sodium acetate buffer added to the column, allowed to gravity flow, and then discarded to clear the column void volume. 5 mL of 250 mM sodium acetate buffer was then added to the column and allowed to gravity elute from the column. Elutions were collected in 0.5 mL fractions and fractions 2-5 were collected, combined, and sterile filtered to produce purified [Zr89]-DFO-Ab1. Purified product was counted on a dose calibrator to confirm activity (Capintech Model CRC-55tR). Samples were evaluated via radioHPLC and radioTLC to ensure greater than 95% chemical and radiochemical purity by both analytical methods. [Zr89]-DFO-Ab1 test articles were then stored at 4° C. until cell studies were ready.

Binding to NCI-H1155 (NSCLC), HCT-116 (colon cancer), and NCI-H520 (NSCLC) cells were assessed. Cells were harvested using trypsin-EDTA (0.25%), washed with cold PBS, and resuspended at a concentration of 5×106 cells/mL in cold staining buffer. 50 μl aliquots containing 10,000 cells were added to the wells of a 96-well, U-bottom plate (ThermoFisher Cat. No. 268200) at a volume of 125 μL and kept on ice (~4° C.).

The stock solution of [Zr89]-DFO-Ab1 was retrieved and diluted to a protein concentration of 200 nM and a radioactive concentration of 32 μCi/mL. A serial dilution series of was then prepared in PBS spanning protein concentrations of 0.0 nM, 0.5 nM, 1.0 nM, SnM, 10 nM, 50 nM, 100 nM, and 200 nM. 125 μL of [Zr89]-DFO-Ab1 was then added to the cells in triplicate and incubated for 30 minutes at 4° C. to induce cell surface binding.

Cells were then retrieved and washed thrice with 500 μL of cold PBS. Cell samples were then transferred to clean microcentrifuge tubes in cold PBS. The tubes were then transferred to a 2480 Wizard2 automatic gamma counter (Revvity, Massachusetts, USA) and analyzed for Zr-89 emissions spanning the 480-950 keV range. Gamma counts were measured for each concentration and normalized by subtracting background counts of untreated control samples.

Activity counts were plotted against concentration in GraphPad Prism and then analyzed with a sigmoidal, dose-response nonlinear regression to determine the cell binding EC50.

Cellular binding of [Zr89]-DFO-Ab1 was assessed following Zr-89 radiolabeling and full chemistry processing in ROR2-positive NCI-H1155, HCT-116 and NCI-H520 cell lines. Binding was assessed across a range (149.4 mCi/mg, 66.1 mCi/mg, 20.7 mCi/mg, and 2.67 mCi/mg) of specific activities (SA) to determine if high SA would impede tumor binding properties due to DAR or radiolytic effects.

### Results

Dose-dependent, nanomolar binding affinity was observed after radiolabeling. Across all specific activities, full nanomolar binding activity was retained and no change in the binding affinity was observed for specific activity ranging up to 149 mCi/mg.

### Example 9: PET/CT Imaging and Dosimetry Analysis with [Zr89]-DFO-Ab1

### Materials and Methods

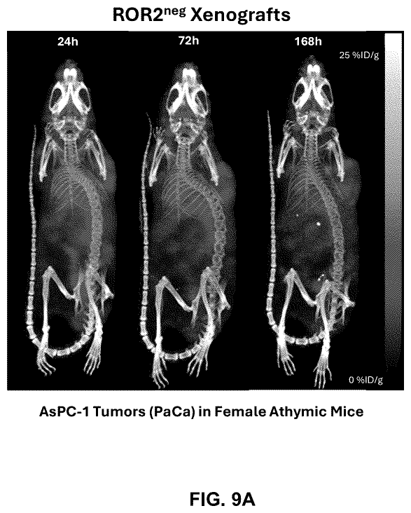

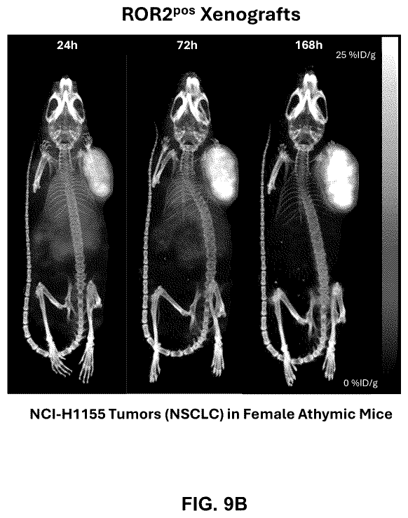

To ascertain the relative biodistribution and non-specific tumor uptake of [Zr89]-DFO-Ab1, microPET/computed tomography (CT) imaging was conducted in female athymic mouse xenograft models of human cancer. Two different models were established: the ROR2-negative tumor line AsPc-1 and the ROR2-positive tumor line NCI-H1155. Tumors were allowed to grow to 150-250 mm3 in athymic nude mice before imaging studies were initiated. Animals (n=4 per model) were injected with ~200 μCi of [Zr89]-DFO-Ab1 in a mass dose of ~18 μg per animal. Animals were serially imaged at timepoints of 24, 72, and 168 hours post-injection using 30- to 60-minute whole-body static scans. PET images were reconstructed and co-registered with the CT images for anatomic overlay, with Zr89 activity quantified as percent injected dose per gram tissue (% ID/g) (FIG. 9A and FIG. 9B).

Initial dosimetry data for [Zr89]-DFO-Ab1 was obtained from the in vivo PET imaging data of the athymic nude mice xenografted with ROR2-negative AsPc-1 tumors. Regions of interest (ROIs) were defined in all images using VivoQuant tools at a uniform voxel size of 0.2 mm3 to identify normal tissues within each animal. The radioactivity uptake within each tissue was quantified as the % ID/g within the defined ROI. Tumor and gastrointestinal tract ROIs were manually drawn to contain the entire mass based upon CT. PET signals were evaluated to ensure all uptake in each organ was included in the relevant ROI. Because AsPc-1% ID/g values were <1%, the tumor ROI and exposure contributions were excluded from the dosimetry analysis. The brain, kidneys, and heart ROIs were defined by fixed-volume phantoms that were manually placed to sample these organs. The bladder ROI was segmented to encompass all activity within the organ. In cases where no activity was evident in the region, the region was defined by placing one spherical volume in the corresponding anatomical location. The femur bone and the periphyseal bone ROIs were manually segmented. The liver, lungs, muscle, and spleen ROIs were defined by placing fixed volume spheres (2 spheres per ROI for the liver, lungs, and muscle and 3 for the spleen) at the corresponding anatomical locations based on CT guidance.

ROI quantification results were converted into time-integrated activity concentrations (TIACs). The TIACs from 0 to 168 hours were calculated using trapezoidal integration. Tail fitting, following the final imaging timepoint, was determined using either exponential decay based on biological clearance from the organ or based on physical decay of the isotope from the final timepoint. Activity in all source organs was assumed to be 0 at 0 hours and to increase linearly to the first measured timepoint at 24 hours. Human dosimetry calculations were performed using organ level internal dose assessment/exponential modeling (OLINDA). Absorbed doses for all human organs, other than urinary bladder contents and whole body, were calculated using the TIACs in the animal organs and applying established medical internal radiation dosimetry (MIRD) scaling models using International Commission on Radiological Protection (ICRP) 89 phantoms for both males and females. Red marrow was calculated by mapping from left ventricle (blood) assuming the concentration ratio between red marrow and blood to be 0.36. In the case of bladder contents and whole body, percent uptake in human was approximated as equivalent to the percent uptake in the animal model. No voiding models were used.

### Results

PET/CT imaging of AsPC-1 tumors shows no specific accumulation of [Ar89]-DFO-Ab1 within the tumors. At all timepoints, the blood pool was the predominant tissue exhibiting Zr89 uptake in the mice. Some non-specific tissue uptake is observed in the ROR2-negative model, but is consistent with highly perfused tissues (e.g., liver, lungs) (FIG. 9A). In contrast, mice bearing NCI-H1155 tumors demonstrated significantly higher tumor uptake for [Zr89]-DFO-Ab1. High uptake was observed at the 24-hour time point and activity profile increased over time until the 168-hour time point. Blood pool remains the predominant non-tumorous tissue of activity, but a strong tumor uptake profile should provide a strong signal-to-noise diagnostic ratio (FIG. 9B1).

The resulting human dosimetry profile for [Zr89]-DFO-Ab1, as shown in Table 7, predicts at mean total body absorbed rates of 0.441 and 0.578 mGy/MBq for adult males and females, respectively. These dosimetry rates are consistent with those resulting from administration ofother Zr89-DFO-containing, antibody-based imaging agents (0.34 to 0.55 mGy/MvBq). As expected for an antibody with a long circulation half-life and stable Zr89 chelation, the greatest organ-specific dosimetry rate and exposure were observed in the heart wall. The results from this initial mouse dosimetry study indicate that clinical administration of [Zr89]-DFO-Ab 1 should result in acceptable whole body and organ-specific radiation exposure profiles.

TABLE 7
Human Dosimetry Calculations (Male)

| Adult Human | Mean Absorbed |
|---|---|
| Male Tissue | Dose (mGy/MBq) |
| Adrenals | 0.405 |
| Brain | 0.089 |
| Esophagus | 0.338 |
| Eyes | 0.185 |
| Gallbladder Wall | 0.480 |
| Heart Wall | 0.624 |
| Kidneys | 0.410 |
| Left colon | 1.766 |
| Liver | 0.399 |
| Lungs | 0.454 |
| Osteogenic Cells | 0.405 |
| Pancreas | 0.442 |
| Prostate | 0.425 |
| Rectum | 1.755 |
| Red Marrow | 0.329 |
| Right colon | 1.344 |
| Salivary Glands | 0.235 |
| Small Intestine | 0.595 |
| Spleen | 0.379 |
| Stomach Wall | 0.380 |
| Testes | 0.233 |
| Thymus | 0.349 |
| Thyroid | 0.286 |
| Total Body | 0.441 |
| Urinary Bladder Wall | 0.412 |

TABLE 8
Human Dosimetry Calculations (Female)

| Adult Human | Mean Absorbed |
|---|---|
| Female Tissue | Dose (mGy/MBq) |
| Adrenals | 0.454 |
| Brain | 0.119 |
| Breasts | 0.274 |
| Esophagus | 0.385 |
| Eyes | 0.233 |
| Gallbladder Wall | 0.549 |
| Heart Wall | 0.710 |
| Kidneys | 0.521 |
| Left colon | 2.109 |
| Liver | 0.475 |
| Lungs | 0.562 |
| Osteogenic Cells | 0.453 |
| Ovaries | 0.587 |
| Pancreas | 0.436 |
| Rectum | 2.140 |
| Red Marrow | 0.418 |
| Right colon | 1.623 |
| Salivary Glands | 0.255 |
| Small Intestine | 0.650 |
| Spleen | 0.472 |
| Stomach Wall | 0.453 |
| Thymus | 0.428 |
| Thyroid | 0.327 |
| Total Body | 0.578 |
| Urinary Bladder Wall | 0.536 |
| Uterus | 0.810 |

### Example 10: Tissue Biodistribution Analysis in Naïve Balb/c Male and Female Mice

### Materials and Methods

[Zr89]-DFO-Ab1 was first synthesized, purified, and formulated in 0.9% o saline with 5 mg/mL gentisic acid. Approximately 100 μL, containing 2 μg of protein and ~20 μCi of radioactivity, was then administered via IV tail-vein injection to 56 BALB/c immunocompetent mice, including 28 males and 28 females (4 animals per sex per timepoint). At 7 post-injection timepoints (1, 4, 24, 72, 96, 168, and 240 hours), animals were euthanized, necropsied, and had a panel of tissues collected. Tissues included the bladder, bone (femur, spinal column, and periphyseal), bone marrow, brain, heart, kidney, large intestine, liver, lung, lymph node, muscle, ovary, pancreas, salivary gland, skin, small intestine, spleen, stomach, testes, thyroid, uterus, and whole blood. Tissues were weighed to determine their respective mass and then analyzed for radioactivity content via gamma counting. Activity concentration of each tissue at each time point was then determined as a percentage of the injected dose per gram of tissue (% ID/g).

The whole blood activity profile, non-decay corrected, was then plotted against the time of tissue collection. The resulting blood-activity curve was then fit using a non-compartmental pharmacokinetic model to determine the effective radioactive half-life of the ARC compared to the physical half-life of the Zr-89 isotope.

The activity profile over time in the various bone tissues will be assessed to determine the rate of free Zr-89 being released due to instability of the DFO-Zr89 chelate under physiological conditions.

### Results

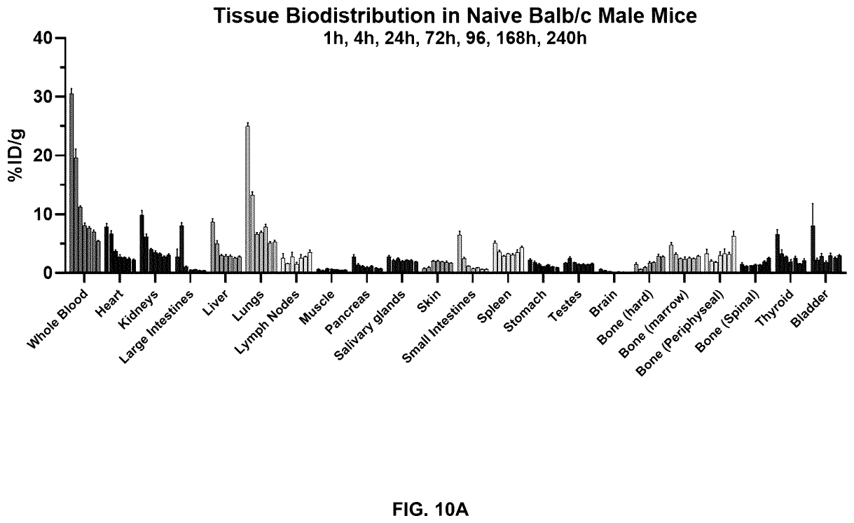

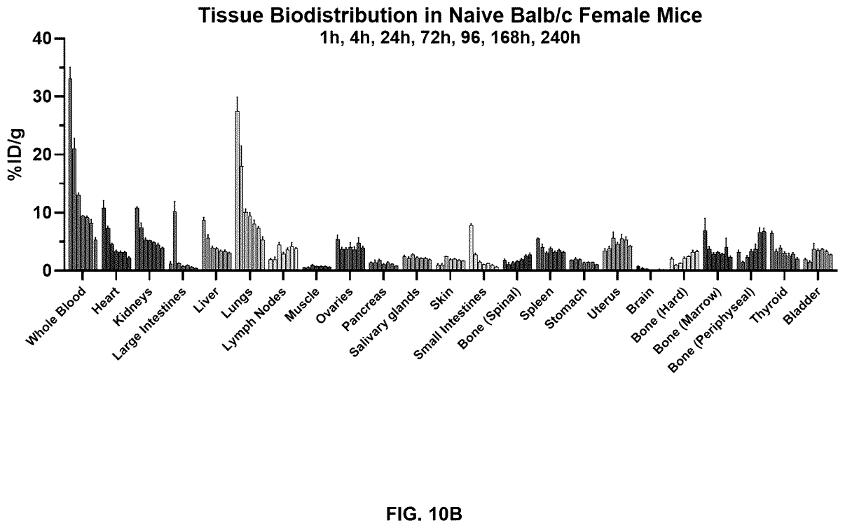

Serial tissue biodistribution was completed in both naïve male and female mice. Whole blood demonstrates the highest activity uptake, followed by proportional levels in highly perfused tissues (e.g., heart, lungs liver). Activity clearance in all tissues follows whole blood clearance kinetics, suggesting no unspecific uptake is observed. Vital tissues show low [Zr89]-DFO-Ab1 uptake (<5%) from 24 hours onward. Key clearance organs for antibodies show low level of uptake (<10%). There is no meaningful difference in tissue uptake between male and female mice (FIGS. 10A and 10B).

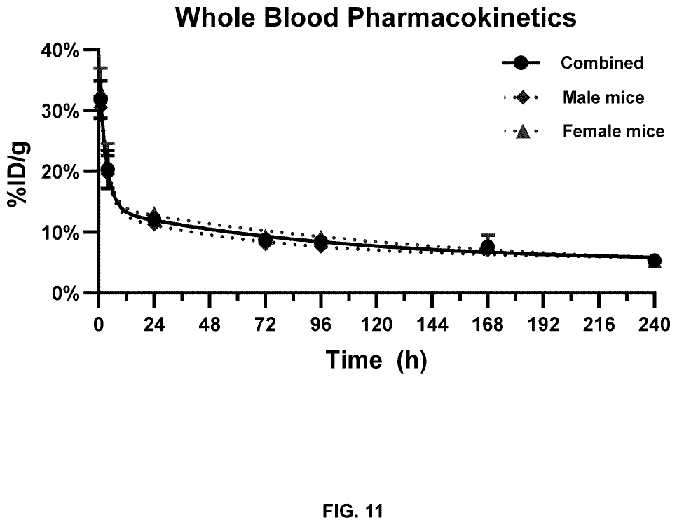

PK analysis of whole blood activity over time shows an effective radioactive half-life of 50.0 hours in male mice and 50.6 hours in female mice, showing no meaningful differentiation in the effective half-life of the ARC depending on the sex of mouse (FIG. 11). At an average of 50.3 hours, the [Zr89]-DFO-Ab1 has an effective half-life that is 36% reduced from the physical half-life of the Zr-89 isotope (78.4 hours).

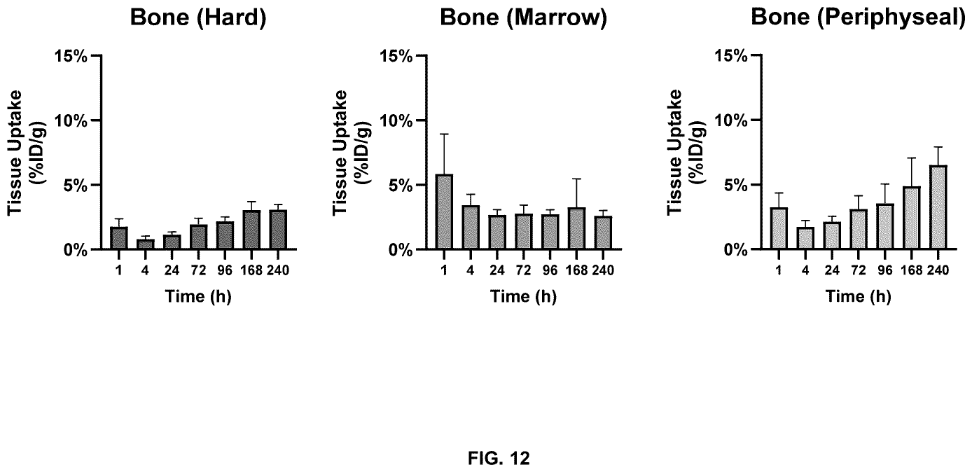

Analysis of the hard bone, bone marrow, and periphyseal bone all serve as biological indications of the amount of free Zr-89 released in vivo from the DFO chelate as Zirconium is a calcium mimetic that is readily incorporated into osteogenic tissue (FIG. 12). Hard bone shows a slight rise in Zr-89 levels from 1% at 4 hours to 3.5% at 240 hours. Bone marrow shows no differential trend in Zr-89 uptake and mostly reflects a consistent vascular exposure of [Zr89]-DFO-Ab. The periphyseal bone, representative of highly osteogenic bone growth, shows uptake increasing from 2% at 4 hours to 7% at 240 hours. The periphyseal and hard bone results do suggest slight instability of the DFO-Zr89 chelate over long periods of time, but not at a rate that will impact imaging sensitivity. When the rate of physical decay of the Zr-89 isotope is taken into account, there is also no meaningful safety concern to the osteogenic tissue by the increase accumulation of Zr-89 over time.

SEQUENCES
Sequences described in the present disclosure are provided below.

| SEQ | | |
|---|---|---|
| ID | | |
| NO: | Name of Sequence | Sequence |
| 1 | Ab1 HC | EVQLVESGGGLVKPGGSLRLSCAASGFTFSTYGVSW |
| | | VRQAPGKGLEWVSTISSGGGYTHYAGSVKGRFTISR |
| | | DNAKNSLYLQMNSLRAEDTAVYYCARHPRDESYALD |
| | | YWGQGTTVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPEAAGGPSVFLFPPKPKD |
| | | TLMISRTPEVTCVVVDVSHEDPEVKENWYVDGVEVH |
| | | NAKTKPREEQYNSTYRVVSVLTVLHQDWLNGKEYKC |
| | | KVSNKALAAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 2 | Ab1/Ab2 LC | EIVMTQSPATLSVSPGERATLSCRASQDVGHYLAWY |
| | | QQKPGQAPRLLIYWASTRATGIPARFSGSGSGTEFT |
| | | LTISSLQSEDFAVYYCQQYNIYPWTFGQGTKVEIKR |
| | | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSENRGEC |
| 3 | Ab1 VH | EVQLVESGGGLVKPGGSLRLSCAASGFTFSTYGVSW |
| | | VRQAPGKGLEWVSTISSGGGYTHYAGSVKGRFTISR |
| | | DNAKNSLYLQMNSLRAEDTAVYYCARHPRDFSYALD |
| | | YWGQGTTVTVSS |
| 4 | Ab1/Ab2 VL | EIVMTQSPATLSVSPGERATLSCRASQDVGHYLAWY |
| | | QQKPGQAPRLLIYWASTRATGIPARFSGSGSGTEFT |
| | | LTISSLQSEDFAVYYCQQYNIYPWTFGQGTKVEIK |
| 5 | Ab1 HCDR1 (IMGT) | GFTFSTYG |
| 6 | Ab1/Ab2/Ab3/Ab4 HCDR2 | ISSGGGYT |
| | (IMGT) | |
| 7 | Ab1 HCDR3 (IMGT/Aho) | ARHPRDESYALDY |
| 8 | Ab1/Ab2/Ab3/Ab4 LCDR1 | QDVGHY |
| | (IMGT) | |
| 9 | Ab1/Ab2/Ab3/Ab4 LCDR2 | WAS |
| | (IMGT) | |
| 10 | Ab1/Ab2/Ab3/Ab4 LCDR3 | QQYNIYPWT |
| | (IMGT/Kabat/Chothia/Aho) | |
| 11 | Ab2 HC | EVQLVESGGGLVKPGGSLRLSCAASGFTFSQYGHSW |
| | | VRQAPGKGLEWVSTISSGGGYTHYAHSVKGRFTISR |
| | | DNAKNSLYLQMNSLRAEDTAVYYCARHPRDFSYAND |
| | | YWGQGTTVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYKC |
| | | KVSNKALAAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 12 | Ab2 VH | EVQLVESGGGLVKPGGSLRLSCAASGFTFSQYGHSW |
| | | VRQAPGKGLEWVSTISSGGGYTHYAHSVKGRFTISR |
| | | DNAKNSLYLQMNSLRAEDTAVYYCARHPRDFSYAND |
| | | YWGQGTTVTVSS |
| 13 | Ab2 HCDR1 (IMGT) | GFTFSQYG |
| 14 | Ab2 HCDR3 (IMGT) | ARHPRDESYANDY |
| 15 | Ab3 HC | EVQLVESGGDLVKPGGSLKLSCAASGFTFSNYGMSW |
| | | VRQTPDKRLEWVATISSGGGYTHYVDSVKGRFTISR |
| | | DNANHILYLQMSSLNSEDTAMYYCARHPRDFSYAMD |
| | | YWGQGTSVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYKC |
| | | KVSNKALPAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 16 | Ab3/Ab4 LC | DIVMTQSHKFMSTSIGDRVSITCKASQDVGHYVAWY |
| | | QQKPGQSPKLLIYWASTRHTGVPDRFTGSGSGTDFT |
| | | LTISNVQSEDLADYFCQQYNIYPWTFGGGSKLAIKR |
| | | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSENRGEC |
| 17 | Ab3/Ab4 VH | EVQLVESGGDLVKPGGSLKLSCAASGFTFSNYGMSW |
| | | VRQTPDKRLEWVATISSGGGYTHYVDSVKGRFTISR |
| | | DNANHILYLQMSSLNSEDTAMYYCARHPRDESYAMD |
| | | YWGQGTSVTVSS |
| 18 | Ab3/Ab4 VL | DIVMTQSHKFMSTSIGDRVSITCKASQDVGHYVAWY |
| | | QQKPGQSPKLLIYWASTRHTGVPDRFTGSGSGTDFT |
| | | LTISNVQSEDLADYFCQQYNIYPWTFGGGSKLAIK |
| 19 | Ab3/Ab4 HCDR1 (IMGT) | GFTFSNYG |
| 20 | Ab3/Ab4 HCDR3 | ARHPRDFSYAMDY |
| | (IMGT) | |
| 21 | Ab4 HC | EVQLVESGGDLVKPGGSLKLSCAASGFTFSNYGMSW |
| | | VRQTPDKRLEWVATISSGGGYTHYVDSVKGRFTISR |
| | | DNANHILYLQMSSLNSEDTAMYYCARHPRDESYAMD |
| | | YWGQGTSVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYKC |
| | | KVSNKALAAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 22 | Ab1 HCDR1 (Kabat) | TYGVS |
| 23 | Ab1 HCDR2 (Kabat) | TISSGGGYTHYAGSVKG |
| 24 | Ab1 HCDR3 | HPRDFSYALDY |
| | (Kabat/Chothia) | |
| 25 | Ab1 LCDR1 | RASQDVGHYLA |
| | (Kabat/Chothia/Aho) | |
| 26 | Ab1 LCDR2 | WASTRAT |
| | (Kabat/Chothia) | |
| 27 | Ab1 HCDR1 (Chothia) | GFTESTY |
| 28 | Ab1 HCDR2 (Chothia) | SSGGGY |
| 29 | Ab1 HCDR1 (AHo) | AASGFTFSTYGVS |
| 30 | Ab1 HCDR2 (AHo) | TISSGGGYTH |
| 31 | Ab1 LCDR2 (AHo) | YWASTRAT |
| 32 | Ab1 HCDR1 (Contact) | STYGVS |
| 33 | Ab1 HCDR2 (Contact) | WVSTISSGGGYTH |
| 34 | Ab1 HCDR3 (Contact) | ARHPRDESYALD |
| 35 | Ab1 LCDR1 (Contact) | GHYLAWY |
| 36 | Ab1 LCDR2 (Contact) | LLIYWASTRA |
| 37 | Ab1 LCDR3 (Contact) | QQYNIYPW |
| 38 | Human ROR2 | MARGSALPRRPLLCIPAVWAAAALLLSVSRTSGEVE |
| | | VLDPNDPLGPLDGQDGPIPTLKGYFLNFLEPVNNIT |
| | | IVQGQTAILHCKVAGNPPPNVRWLKNDAPVVQEPRR |
| | | IIIRKTEYGSRLRIQDLDTTDTGYYQCVATNGMKTI |
| | | TATGVLFVRLGPTHSPNHNFQDDYHEDGFCQPYRGI |
| | | ACARFIGNRTIYVDSLQMQGEIENRITAAFTMIGTS |
| | | THLSDQCSQFAIPSFCHFVFPLCDARSRTPKPRELC |
| | | RDECEVLESDLCRQEYTIARSNPLILMRLQLPKCEA |
| | | LPMPESPDAANCMRIGIPAERLGRYHQCYNGSGMDY |
| | | RGTASTTKSGHQCQPWALQHPHSHHLSSTDFPELGG |
| | | GHAYCRNPGGQMEGPWCFTQNKNVRMELCDVPSCSP |
| | | RDSSKMGILYILVPSIAIPLVIACLFFLVCMCRNKQ |
| | | KASASTPQRRQLMASPSQDMEMPLINQHKQAKLKEI |
| | | SLSAVREMEELGEDRFGKVYKGHLFGPAPGEQTQAV |
| | | AIKTLKDKAEGPLREEFRHEAMLRARLQHPNVVCLL |
| | | GVVTKDQPLSMIFSYCSHGDLHEFLVMRSPHSDVGS |
| | | TDDDRTVKSALEPPDFVHLVAQIAAGMEYLSSHHVV |
| | | HKDLATRNVLVYDKLNVKISDLGLFREVYAADYYKL |
| | | LGNSLLPIRWMAPEAIMYGKFSIDSDIWSYGVVLWE |
| | | VFSYGLQPYCGYSNQDVVEMIRNRQVLPCPDDCPAW |
| | | VYALMIECWNEFPSRRPRFKDIHSRLRAWGNLSNYN |
| | | SSAQTSGASNTTQTSSLSTSPVSNVSNARYVGPKQK |
| | | APPFPQPQFIPMKGQIRPMVPPPQLYVPVNGYQPVP |
| | | AYGAYLPNFYPVQIPMQMAPQQVPPQMVPKPSSHHS |
| | | GSGSTSTGYVTTAPSNTSMADRAALLSEGADDTQNA |
| | | PEDGAQSTVQEAEEEEEGSVPETELLGDCDTLQVDE |
| | | AQVQLEA |
| 39 | GGFG linker | GGFG |
| 40 | Ab1 HCDR1 | TY |
| 41 | Ab1 LCDR1 | GHY |

1. An immunoconjugate comprising a means for binding to human ROR2 and a radionuclide conjugated to the means.

2. The immunoconjugate of claim 1, wherein the means is an anti-ROR2 antibody or an antigen-binding portion thereof,
optionally wherein the antibody or antigen-binding portion competes or cross-competes for binding to human ROR2, or binds to the same human ROR2 epitope, as an antibody that comprises a heavy chain (HC) and a light chain (LC) comprising
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively;
further optionally wherein the antigen-binding portion is a Fab, F(ab)₂, or scFv.

3. The immunoconjugate of claim 2, wherein the antibody or antigen-binding portion comprises
HCDR1 comprising SEQ ID NO: 40;
HCDR2 comprising SEQ ID NO: 28;
HCDR3 comprising SEQ ID NO: 24;
LCDR1 comprising SEQ ID NO: 41;
LCDR2 comprising SEQ ID NO: 9; and
LCDR3 comprising SEQ ID NO: 37.

4. The immunoconjugate of claim 2, wherein the antibody or antigen-binding portion comprises heavy chain complementarity-determining region (CDR) 1-3 (HCDR1-3) and light chain CDR1-3 (LCDR1-3) amino acid sequences of
a) SEQ ID NOs: 5, 6, 7, 8, 9, and 10, respectively;
b) SEQ ID NOs: 13, 6, 14, 8, 9, and 10, respectively; or
c) SEQ ID NOs: 19, 6, 20, 8, 9, and 10, respectively.

5. (canceled)

6. The immunoconjugate of claim 4, wherein the antibody or antigen-binding portion comprises heavy chain variable domain (V_{H}) and light chain variable domain (V_{L}) amino acid sequences of
a) SEQ ID NOs: 3 and 4, respectively;
b) SEQ ID NOs: 12 and 4, respectively; or
c) SEQ ID NOs: 17 and 18, respectively.

7. The immunoconjugate of claim 4, wherein the antibody is of isotype IgG,
optionally wherein the antibody is of isotype subclass IgG₁, IgG₂, IgG₃, or IgG₄,
further optionally wherein the Fc region of the antibody comprises one or more mutations that reduce effector function.

8-9. (canceled)

10. The immunoconjugate of claim 4, wherein the antibody comprises HC and LC amino acid sequences of
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively;
optionally wherein the HC amino acid sequence lacks the C-terminal lysine.

11. (canceled)

12. The immunoconjugate of claim 4, wherein the radionuclide is zirconium-89, optionally conjugated to one or more lysine residues in the antibody or antigen-binding portion by a metal chelation linker selected from 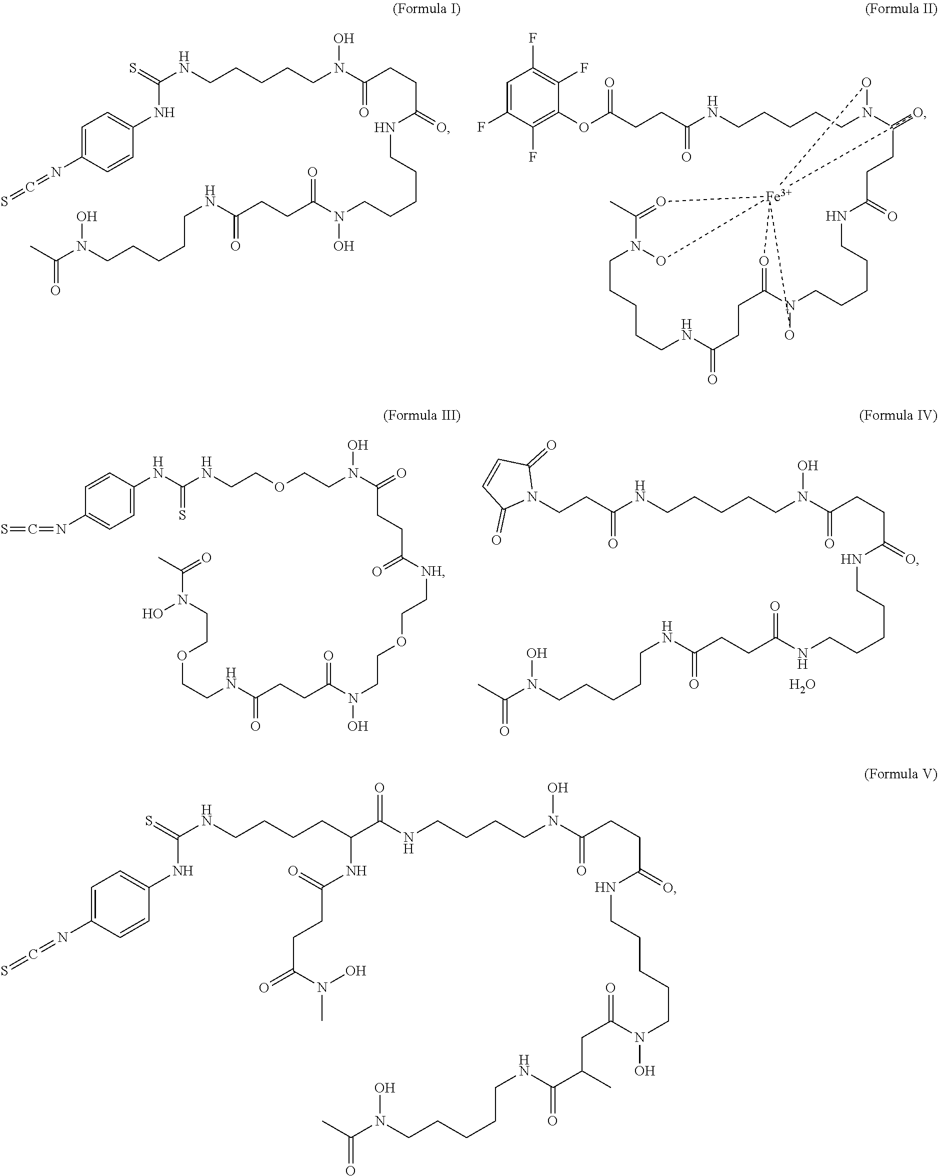 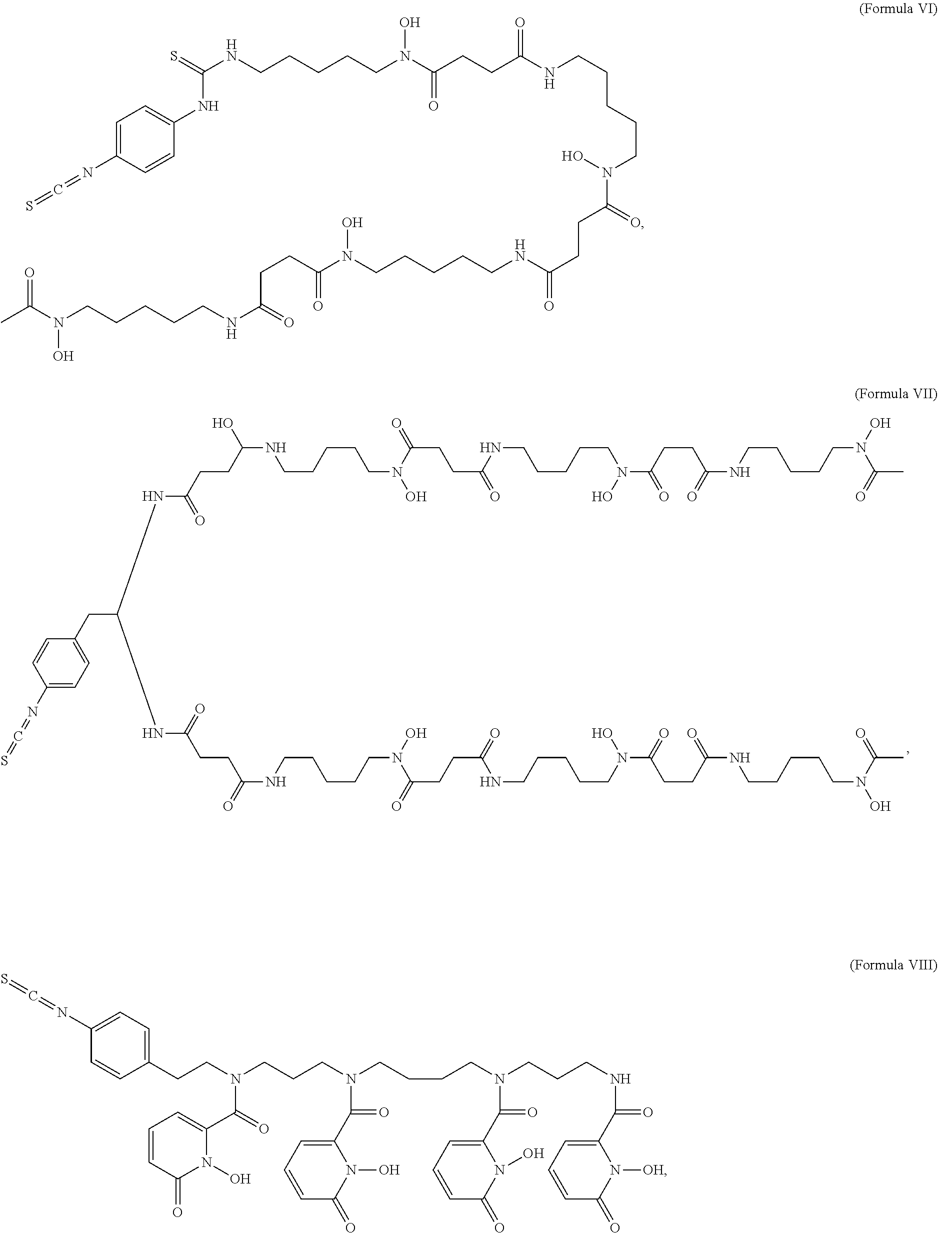 [SMILES: CC(=O)N(O)CCCCCNC(=O)CCC(=O)N(O)CCCCCNC(=O)CCC(=O)N(O)CCCCCNC(=O)CCC(=O)N(O)CCCCCNC(=S)Nc1ccc(N=C=S)cc1 ; CC(=O)N(O)CCCCCNC(=O)CCC(=O)N(O)CCCCCNC(=O)CCC(=O)N(O)CCCCCNC(=O)CCC(=O)NC(Cc1ccc(N=C=S)cc1)NC(=O)CCC(O)NCCCCCN(O)C(=O)CCC(=O)NCCCCCN(O)C(=O)CCC(=O)NCCCCCN(O)C(C)=O ; O=C(NCCCN(CCCCN(CCCN(CCc1ccc(N=C=S)cc1)C(=O)c1cccc(=O)n1O)C(=O)c1cccc(=O)n1O)C(=O)c1cccc(=O)n1O)c1cccc(=O)n1O]
or dodecane tetraacetic acid (DOTA) or an active ester thereof, 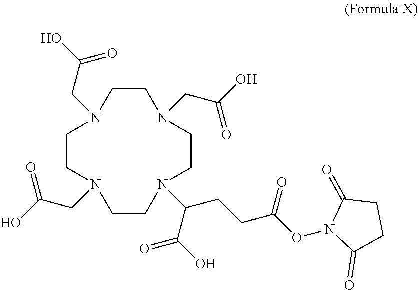 [SMILES: O=C(O)CN1CCN(CC(=O)O)CCN(C(CCC(=O)ON2C(=O)CCC2=O)C(=O)O)CCN(CC(=O)O)CC1]
or 1,4,7,10-tetraazacyclododececane, 1-(glutaric acid)-4,7,10-triacetic acid (DOTAGA), or an active ester thereof, and

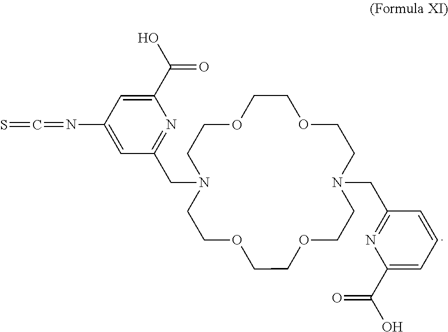 [SMILES: O=C(O)c1cccc(CN2CCOCCOCCN(Cc3cc(N=C=S)cc(C(=O)O)n3)CCOCCOCC2)n1]

13. The immunoconjugate of claim 12, comprising one of the following structures, wherein Ab is the anti-ROR2 antibody or antigen-binding portion:

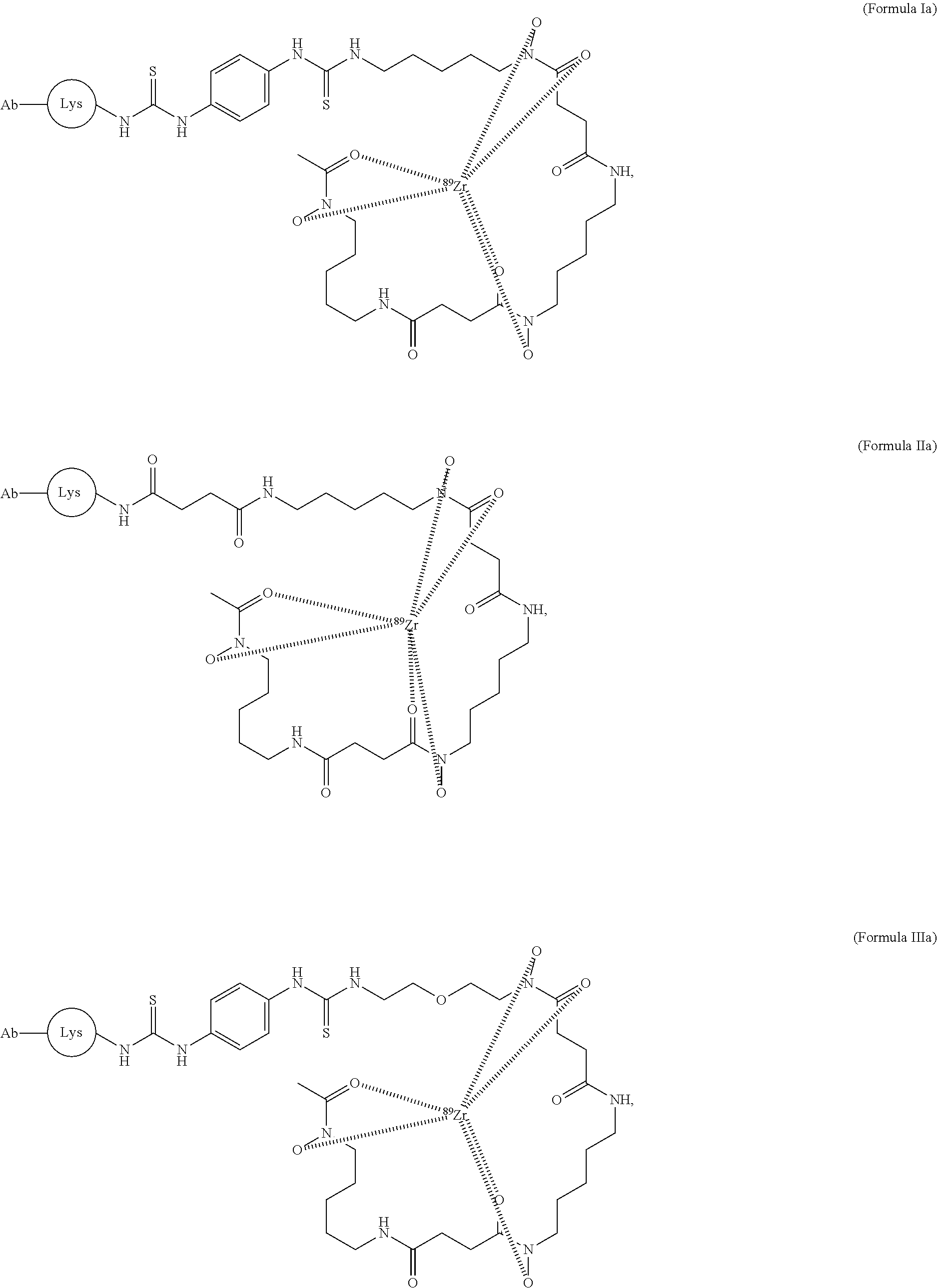

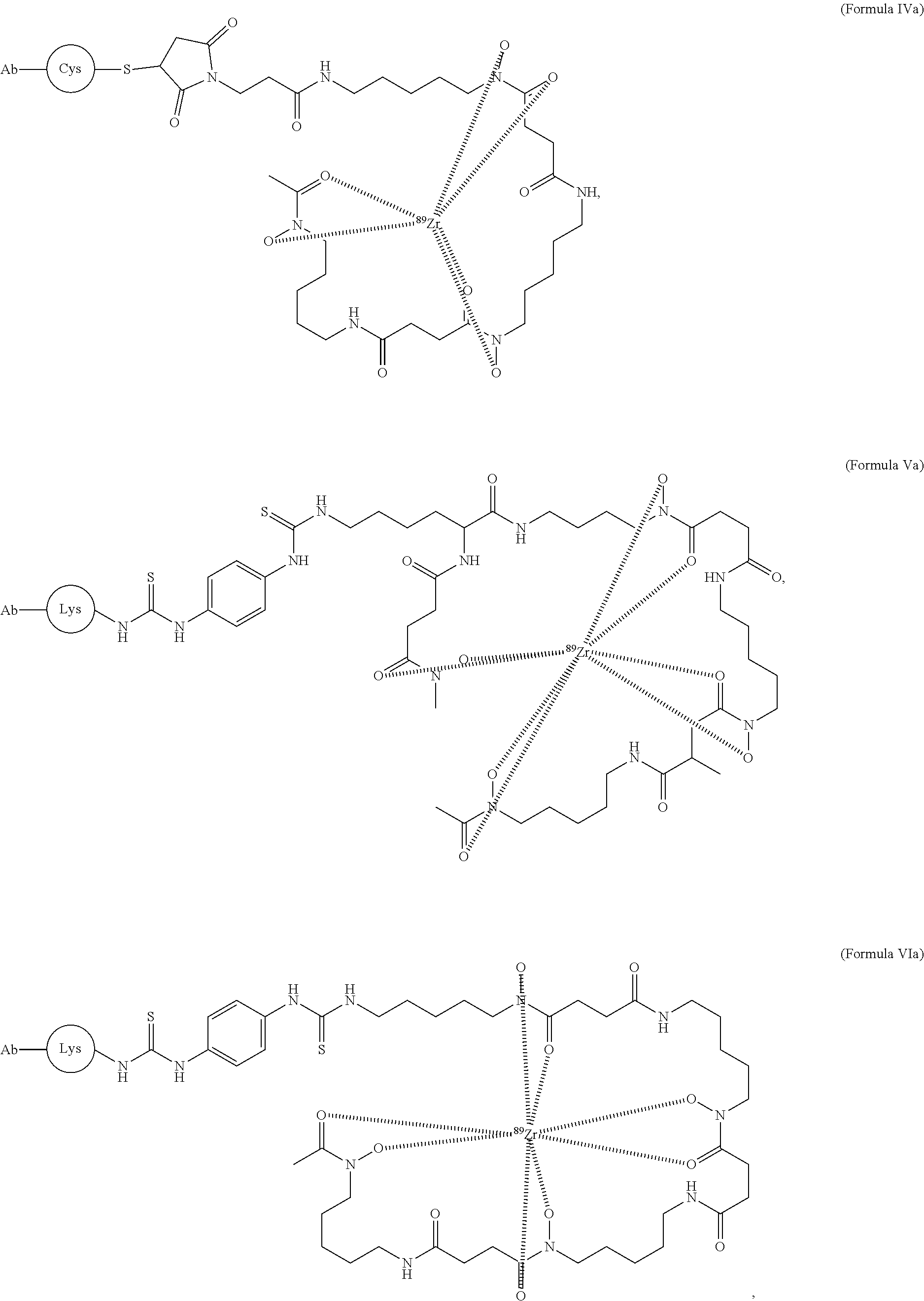

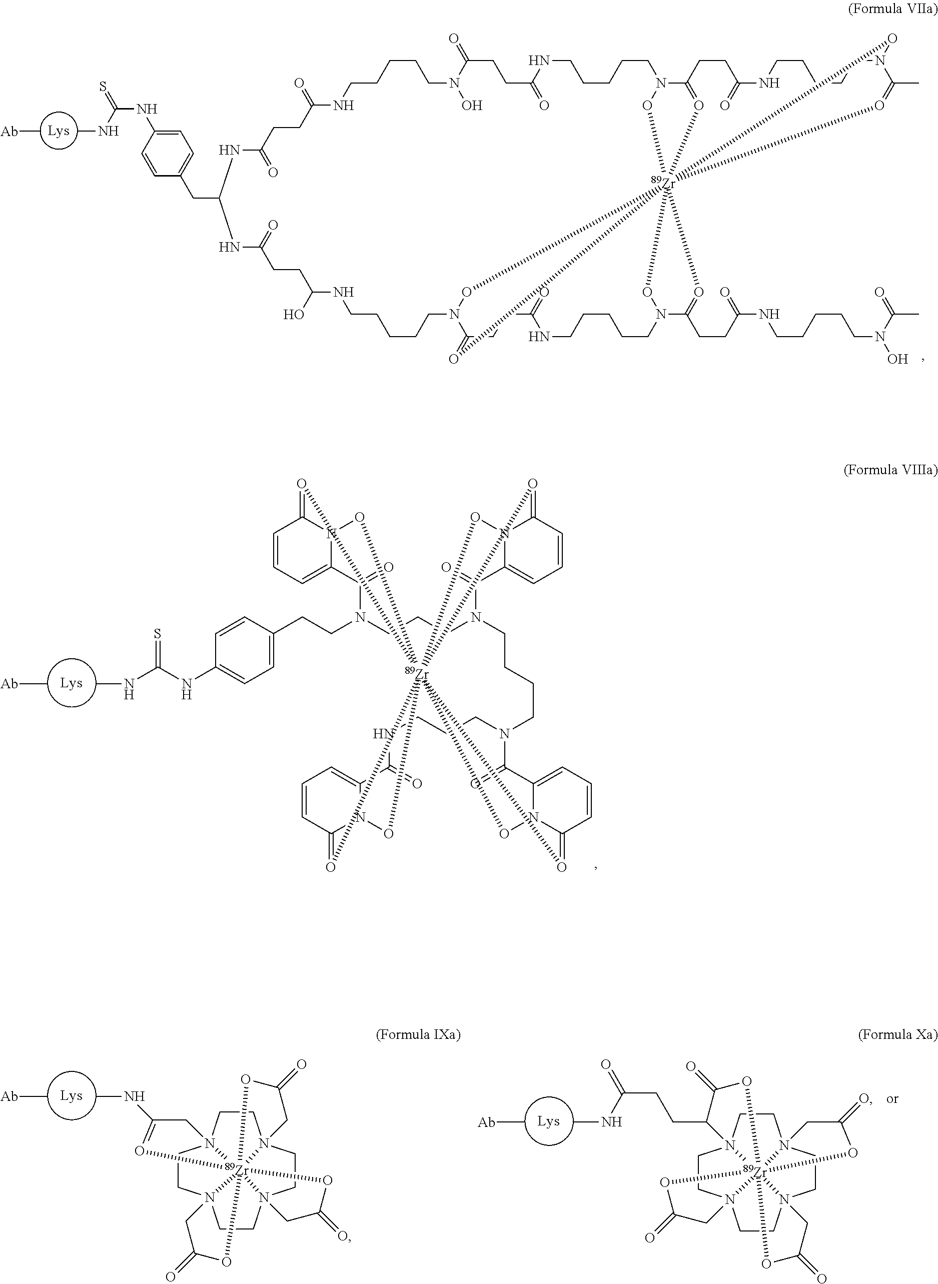

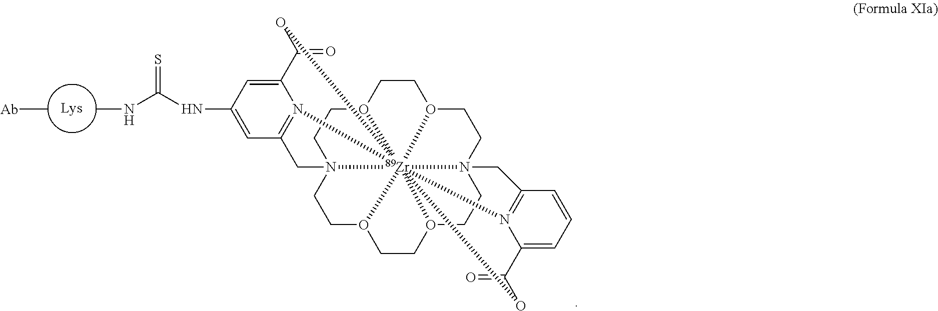

14. The immunoconjugate of claim 4, wherein the radionuclide is a halogen, optionally fluorine-18, iodine-124, iodine-125, iodine-131, or astatine-211, optionally conjugated to one or more lysines in the antibody or antigen-binding portion using a halogenation linker represented by 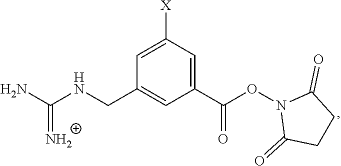
wherein X is ¹⁸F, ⁷⁶Br, ⁷⁷Br, ⁷⁸Br, ¹²³I, ¹²⁴I, ¹²⁵I, ¹³¹I, or ²¹¹At.

15. The immunoconjugate of claim 14, comprising one of the following structures, wherein Ab is the anti-ROR2 antibody or antigen-binding portion:

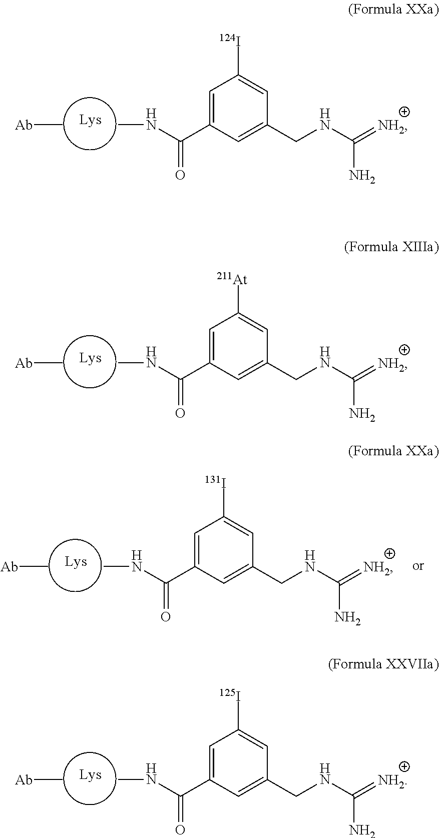

16. The immunoconjugate of claim 4, wherein the immunoconjugate emits alpha-particles, optionally wherein the immunoconjugate has one of the following structures, wherein Ab is the anti-ROR2 antibody or antigen-binding portion:

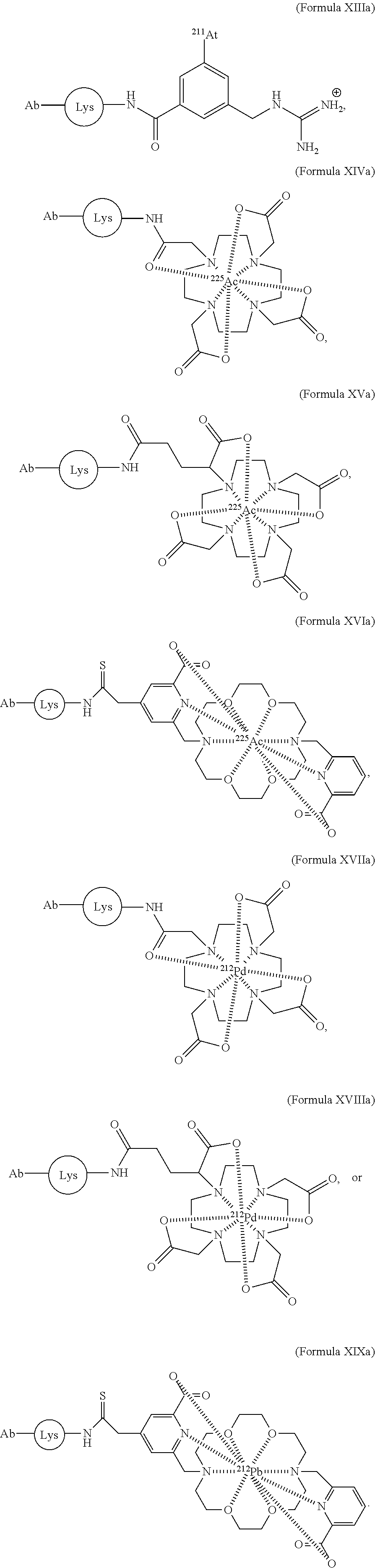

17. The immunoconjugate of claim 4, wherein the immunoconjugate emits beta-particles, optionally wherein the immunoconjugate has one of the following structures, wherein Ab is the anti-ROR2 antibody or antigen-binding portion:

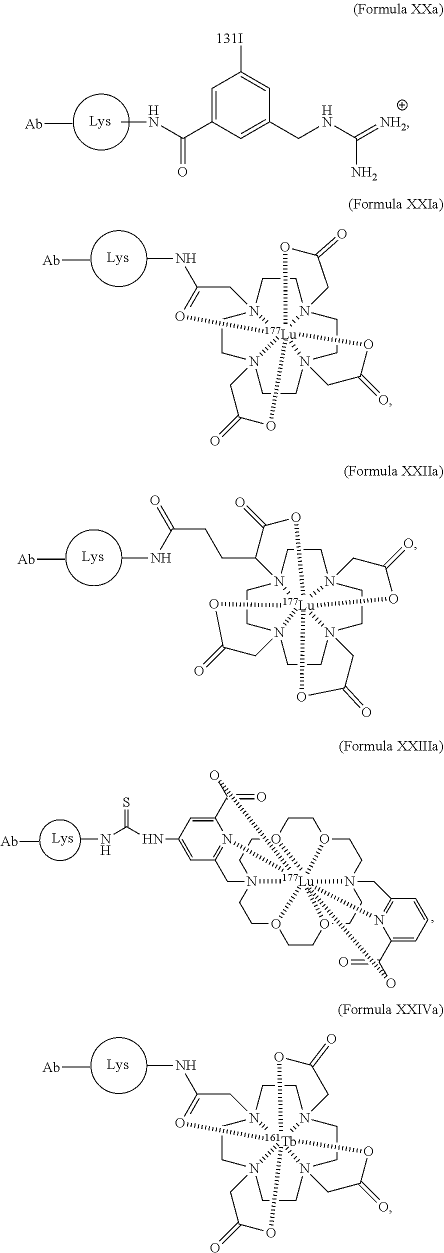

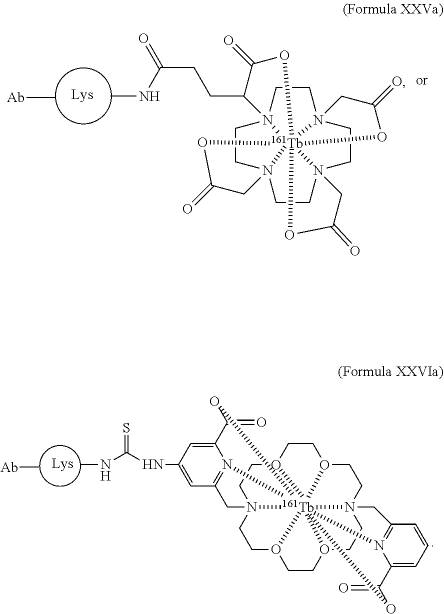

18. The immunoconjugate of claim 4, wherein the immunoconjugate emits Auger electrons, optionally wherein the immunoconjugate has the following structure, wherein Ab is the anti-ROR2 antibody or antigen-binding portion:

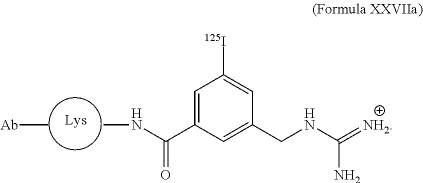

19. The immunoconjugate of claim 4, further conjugated to a cytotoxic drug, optionally wherein the cytotoxic drug is conjugated to cysteine residues on the antibody or antigen-binding portion through a linker.

20. A pharmaceutical composition comprising the immunoconjugate of claim 4 and a pharmaceutically acceptable excipient.

21. A method of diagnosing ROR2-positive cancer in a patient in need thereof, comprising:
introducing a diagnostically effective amount of the immunoconjugate of claim 4 into the patient, and
imaging the radioactivity emitting from the patient.

22. A method of treating cancer in a patient in need thereof, comprising administering to the patient a therapeutically effective amount of the immunoconjugate of claim 4,
optionally wherein the cancer expresses ROR2,
further optionally wherein the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma.

23-24. (canceled)

25. The method of claim 22, further comprising administering to the patient an additional therapeutic agent, optionally wherein the additional therapeutic agent is selected from the group consisting of an immunomodulatory agent, a chemotherapeutic agent, a radionuclide agent, a nuclear imaging agent, an anti-neoplastic agent, an anti-angiogenic agent, or a tumor vaccine.

26-28. (canceled)

29. A method of making an immunoconjugate of claim 4, comprising:
providing an anti-ROR2 antibody or an antigen-binding portion thereof, and
conjugating a radionuclide to the antibody or antigen-binding portion.


SEQUENCE LISTING (41 sequences)

SEQ ID NO: 1 - protein, 450 aa, synthetic construct
EVQLVESGGGLVKPGGSLRLSCAASGFTFSTYGVSWVRQAPGKGLEWVSTISSGGGYTHYAGSVKGRFTISRDNAKNSLYLQMNSLRAEDTAVYYCARHPRDFSYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
EIVMTQSPATLSVSPGERATLSCRASQDVGHYLAWYQQKPGQAPRLLIYWASTRATGIPARFSGSGSGTEFTLTISSLQSEDFAVYYCQQYNIYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, synthetic construct
EVQLVESGGGLVKPGGSLRLSCAASGFTFSTYGVSWVRQAPGKGLEWVSTISSGGGYTHYAGSVKGRFTISRDNAKNSLYLQMNSLRAEDTAVYYCARHPRDFSYALDYWGQGTTVTVSS

SEQ ID NO: 4 - protein, 107 aa, synthetic construct
EIVMTQSPATLSVSPGERATLSCRASQDVGHYLAWYQQKPGQAPRLLIYWASTRATGIPARFSGSGSGTEFTLTISSLQSEDFAVYYCQQYNIYPWTFGQGTKVEIK

SEQ ID NO: 5 - protein, 8 aa, synthetic construct
GFTFSTYG

SEQ ID NO: 6 - protein, 8 aa, synthetic construct
ISSGGGYT

SEQ ID NO: 7 - protein, 13 aa, synthetic construct
ARHPRDFSYALDY

SEQ ID NO: 8 - protein, 6 aa, synthetic construct
QDVGHY

SEQ ID NO: 9 - skipped

SEQ ID NO: 10 - protein, 9 aa, synthetic construct
QQYNIYPWT

SEQ ID NO: 11 - protein, 450 aa, synthetic construct
EVQLVESGGGLVKPGGSLRLSCAASGFTFSQYGHSWVRQAPGKGLEWVSTISSGGGYTHYAHSVKGRFTISRDNAKNSLYLQMNSLRAEDTAVYYCARHPRDFSYANDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 120 aa, synthetic construct
EVQLVESGGGLVKPGGSLRLSCAASGFTFSQYGHSWVRQAPGKGLEWVSTISSGGGYTHYAHSVKGRFTISRDNAKNSLYLQMNSLRAEDTAVYYCARHPRDFSYANDYWGQGTTVTVSS

SEQ ID NO: 13 - protein, 8 aa, synthetic construct
GFTFSQYG

SEQ ID NO: 14 - protein, 13 aa, synthetic construct
ARHPRDFSYANDY

SEQ ID NO: 15 - protein, 450 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, synthetic construct
DIVMTQSHKFMSTSIGDRVSITCKASQDVGHYVAWYQQKPGQSPKLLIYWASTRHTGVPDRFTGSGSGTDFTLTISNVQSEDLADYFCQQYNIYPWTFGGGSKLAIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 120 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSS

SEQ ID NO: 18 - protein, 107 aa, synthetic construct
DIVMTQSHKFMSTSIGDRVSITCKASQDVGHYVAWYQQKPGQSPKLLIYWASTRHTGVPDRFTGSGSGTDFTLTISNVQSEDLADYFCQQYNIYPWTFGGGSKLAIK

SEQ ID NO: 19 - protein, 8 aa, synthetic construct
GFTFSNYG

SEQ ID NO: 20 - protein, 13 aa, synthetic construct
ARHPRDFSYAMDY

SEQ ID NO: 21 - protein, 450 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, synthetic construct
TYGVS

SEQ ID NO: 23 - protein, 17 aa, synthetic construct
TISSGGGYTHYAGSVKG

SEQ ID NO: 24 - protein, 11 aa, synthetic construct
HPRDFSYALDY

SEQ ID NO: 25 - protein, 11 aa, synthetic construct
RASQDVGHYLA

SEQ ID NO: 26 - protein, 7 aa, synthetic construct
WASTRAT

SEQ ID NO: 27 - protein, 7 aa, synthetic construct
GFTFSTY

SEQ ID NO: 28 - protein, 6 aa, synthetic construct
SSGGGY

SEQ ID NO: 29 - protein, 13 aa, synthetic construct
AASGFTFSTYGVS

SEQ ID NO: 30 - protein, 10 aa, synthetic construct
TISSGGGYTH

SEQ ID NO: 31 - protein, 8 aa, synthetic construct
YWASTRAT

SEQ ID NO: 32 - protein, 6 aa, synthetic construct
STYGVS

SEQ ID NO: 33 - protein, 13 aa, synthetic construct
WVSTISSGGGYTH

SEQ ID NO: 34 - protein, 12 aa, synthetic construct
ARHPRDFSYALD

SEQ ID NO: 35 - protein, 7 aa, synthetic construct
GHYLAWY

SEQ ID NO: 36 - protein, 10 aa, synthetic construct
LLIYWASTRA

SEQ ID NO: 37 - protein, 8 aa, synthetic construct
QQYNIYPW

SEQ ID NO: 38 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 39 - protein, 4 aa, synthetic construct
GGFG

SEQ ID NO: 40-41 - skipped (2 entries)